SEQUENCE LISTING (68 sequences)

SEQ ID NO: 1 - DNA, 7382 bp, Mus musculus
  CDS (133)..(6531)
ccccccgcgcagagcatccctgctgcagcgcagcaagacccggggctggcagccccaggaatccctagctgcttcgcagggataaaggactgaagttcttggaggagcgagtccaactcttcaagctgaactatgaccactttactcttggtctttgtgactctgagggtcatcgctgcagtgatctcagaagaagttccagaccatgacaactcactgagcgtgagcatccctcaaccatccccattgaaggtcctcctagggtcttccctcaccatcccctgctacttcatcgaccccatgcatcctgtgaccactgccccctccactgcccccctcaccccaagaatcaagtggagccgtgtttccaaggaaaaggaggtggtactgctggtggccactgaaggacaggttcgagtcaacagcatctaccaagacaaggtgtcgctccccaactatccagccatccccagcgatgctaccttggagatccagaaccttcgctccaatgactctgggatctaccgctgtgaagtgatgcacggcatcgaggacagcgaagccaccctggaggtcatagtgaaaggtattgtgttccactacagagctatttccacacgctacaccctggactttgatcgagcacagcgggcttgcctacagaacagcgccatcatcgccaccccagaacaactgcaggctgcctatgaggatggcttccaccagtgcgatgcaggctggctggctgaccagacagtcagataccccatccacacgccccgggaaggttgctatggtgacaaggacgagttccctggagtgagaacctacggaatccgggacaccaatgagacctatgatgtgtactgcttcgctgaggagatggagggtgaggtcttttatgcgacatccccagagaaattcaccttccaggaggcagccaacgagtgccggaggctgggggcacgcttggccaccacaggccagctctacctggcctggcagggcggtatggacatgtgcagcgctggctggctggcggaccgcagcgtgcgctaccccatctccaaggctcggcccaactgcgggggcaacctcctgggtgtaaggactgtctatctacacgccaaccagacaggctatcctgatccctcatcccgatatgacgccatctgttacacaggtgaagattttgtagacatcccagaaaacttcttcggggtgggtggtgaagacgacatcaccatccagacggtgacctggccagacctggagctgcccttgccccgtaatgtcacagagggagaggccctgggcagcgtgatcctcacggcaaagcccatctttgacctgtcccccactatctcagagccgggggaggccctcacacttgcccctgaagtggggagcacagccttcccagaggctgaggagagaactggagaagccaccagaccctggggctttcctgcagaagtcacacgtgggccggactctgccactgcctttgccagtgaggacctggtagtgcgagtgaccatctctccaggtgcagctgaagtccctggtcagccccacttgccagggggagttgtattccactatcgcccaggctccaccagatactctctgacatttgaggaggcacagcaggcctgcatgcacaccggggccatcattgcctctcctgagcagctccaagctgcctatgaggcaggctatgagcagtgtgatgctggctggctgcaggaccagaccgtcagataccccattgtgagcccacgaaccccatgtgtgggtgacaaagacagcagcccaggagtcaggacctaccgcgtgcgcccatcatcagaaacctatgatgtctactgctacgtggacaagcttgagggggaagtgttcttcgccacacgcctcgagcagttcaccttccaggaagcgcgggccttctgtgcggctcaaaatgccaccctggcctccaccggccagctctatgctgcctggagccagggtctggacaagtgctatgctggctggctggcagatggcaccctccgataccccatcatcacccctcggcctgcctgtggtggggacaaacctggcgtgagaactgtctacctctaccccaaccaaaccggcctccctgacccactgtcaaagcaccatgccttctgcttccgaggtgtgtcagtggcgccctctccaggagaagaagagggtagtacacccacatcaccctctgacatagaggactggatcgtcactcaggtggggcctggtgtggatgctgtccccttggagccaaagacaacagaagtgccatatttcaccactgagccaagaaaacagactgaatgggagccagcctacaccccagtgggcacatccccacagccagggatccctcccacatggcttcccaccctcccagcagcagaggaacacacagaaagcccctctgcctctgaagagccctctgcctcagcagtcccttccacctcagaggagccatacacatcttcatttgcagtgccgagcatgacagagctgccaggctctggggaggcgtcgggcgcacctgacctcagtggtgacttcacaggcagtggagatgcttcaggacgccttgactccagtgggcagccttcagggggcattgaaagtggccttccctcaggtgaccttgactccagtggcctcagccccacagtgagctcaggcctgcctgtagaaagtggttctgcctcaggagatggagaagtcccctggtcccatactcccacagttggcaggttgccctctggaggtgagagccccgaaggctctgcctctgcctctggaacaggagaccttagtgggctgccttcaggaggagaaattacagaaacttctacttctggggcagaagaaaccagtggacttccttctggaggagacggtctagaaacttctacctctggagtagatgatgtcagtggaattcctactggaagagaaggtctagagacttctgcctctggagtagaggacctcagtggacttccttctggagaagaaggttcagaaacatctacctctggaatagaggacatcagtgtacttccaactggaggagaaagtctagaaacctctgcttctggagtgggagacttgagtggacttccctcaggaggagaaagtctagaaacatctgcttcaggtgcagaggatgtcactcagcttcctactgaaagaggaggtctagagacttctgcctctggagtagaagacatcactgttcttcctactggaagagaaagtctagaaacttctgcctctggagtagaggatgtcagtggacttccttctggaagagaaggtctagagacttctgcctctggaatagaggacattagtgtgtttcctactgaagcagaaggtctggacacttctgcctctgggggatatgttagtgggattccttctggaggagatggtacagaaacctctgcttctggagtagaggatgtgagtggtcttccatctggaggagagggtctagaaacttctgcctctggagtggaagatcttggtccttctactagagatagtctagagacatctgcttcaggagtagatgttactgggtttccttctggaagaggggacccagagacctctgtttctggggtaggtgatgacttcagtggacttccttctggaaaagaaggcctggagacctcagcttctggagctgaggacctcagtggcttgccctctggaaaagaagacttggtagggtctgcttctggggccttggactttggcaaactacctcctggaactctaggaagtggtcaaactccagaagtaaatggctttccctctggatttagtggtgagtattctggagcagacattggaagtggcccatcctctggcctgcctgactttagtggacttccatctggctttccaactgtctcccttgtggacagtaccttagtggaagtgatcacagccaccacttccagtgaactggaaggaagggggaccattggcatcagtggttcaggagaagtatcagggctgcccctgggtgaattggacagtagtgcggacattagtggtctcccttcaggaactgaactcagtggccaagcatctggatctcctgatagcagtggagaaacatctggattttttgatgttagtggacagccatttgggtcttctggcgtcagcgaggaaacatctgggattcctgaaatcagtgggcagccatcagggactcctgacaccactgcgacatctggagtgactgagcttaatgaactgtcctctggacaaccagatgtcagtggagatgggtctggaattctctttggcagtggacagtcctctggtataacatctgtgagtggagaaacctctgggatttctgatctcagtgggcagccctcagggttcccagtgttcagtggaacagcaaccagaacccctgacctggcttctggcaccataagtggcagtggagagtcttctggcattacatttgtggacaccagttttgttgaagtgacccctaccacatttagggaagaagaagggttaggatctgtggaactcagtggctttccttctggggagacggaactgtctggcacatctgggacggtggacgtcagtgaacaatcttctggagcaattgattccagtggactcacatcccccactccagagttcagtggcctcccaagtggagtagctgaggtcagtggtgaattctctggagttgagactgggagcagcttgccctcaggagcatttgatggcagtggacttgtctcaggtttccccactgtgtctcttgtagacagaactttggtggaatctataactcaggctcctactgctcaagaagctggagaaggaccttcgggcattttggaattcagtggtgcccattctgggacaccagacatatctggggagctttctgggtctctggacctaagcacattgcagtctgggcagatggaaaccagcacggagacaccaagctctccatattttagtggagacttttccagcaccactgatgtaagtggagaatccatagctgccacaactggcagtggggaaagctctgggcttccggaagttactttaaacacctcagagttagtggagggtgtgactgaacccactgtttcccaggaacttggccatggtccttctatgacatacatctcccggctctctgaggccagtggggacgcctcagcatccggggaccttggtggcgctgtaacaaacttcccagggtctggagtagaagcttcagtcccagaagccagcagtgacctgtctgcttaccctgaggctggtgtgggagtgtctgctgcccctgaggccagcagtaaactgtctgagttcccagatctgcatggaatcacttctgccttccatgaaacagatctggaaatgacaaccccaagcacagaggtaaacagcaacccatggacctttcaggaaggcaccagggagggatcggccgctccggaagtgagtggagaatctagcactacctccgacatagacacaggcacttcaggggtgccttctgccacacccatggcttctggagacaggactgaaatcagcggagaatggtctgatcacacctcagaggtgaatgttgccatcagcagcaccatcacagagtccgagtgggcccagcctacccggtaccctacagagacacttcaagaaatcgaatccccaaatccctcatactcaggagaggagacccagacagcagaaacaaccatgtccctgacagatgcccccaccctctcttcttcagaagggtcaggggagacggagtcaaccgttgcagaccaggagcaatgtgaggaggggtggaccaagttccagggtcactgttaccgccactttcctgaccgagagacctgggtggatgcggagagacggtgtcgggagcagcagtcacatctgagcagcattgtcactcctgaggaacaggagttcgtcaacaaaaatgctcaagactaccagtggatcggtctgaatgacaggactatcgaaggggacttccgctggtctgacggacactctctgcaatttgagaagtggcgtccaaaccagcctgacaacttctttgccaccggagaggactgtgtggtgatgatctggcatgagagaggcgaatggaacgacgtcccctgcaattaccagctgcccttcacgtgtaaaaagggcaccgtggcctgtggagaccccccagtggtggagcatgctagaaccctcgggcagaagaaagatcgctacgagatcagctccctggtgcggtaccagtgcactgagggctttgtccagcgccacgtgcccaccatccggtgccagcccagcgggcactgggaagagcctcgaatcacctgcacagaccccaacacctacaagcacaggctacagaagcggacgatgagacccacacggaggagccgccccagcatggcccactgagaggagcttccataatgtgcccaggatgctgagcccagcggccagccaggctgaccgtgcatcccacccacatggtgtcttcttgtcgctttttgtcatataaggaatccattaaagaaggaaaaaaaacccacattgtgtgtgccccactccccgcatcacccaaactgcatctaatttatccccttctcccccccccccaaaaaaaaaaagaaaagaaatgcccaagcaagtcactgtaaccccgtggtacctcagctttgagacttttttgatttccccacccgtgccctttcccagggaccagtgcaggcacagggtgagaaggtaaggggttaaattaaataaagaagattcttttttgttgttgtttcctgactttgcccaagaacagtacaatggttggttacttcgcctccagggagagctaggggcccagaggaggagctgaaagaagacaggaccaggaagggaggagtagggcggtcacagagcttggaggactcagaagaggcaaagtacagcttagtgtgttggacaaaaggaattgaggtctgtgccatctgtgagggaaggacctggggcagggacaggctggctgaagagaagcagagccctctcctagggacctttcatctgggtcaaccaacctagcaggtgtcatttctcggctggactgggtaggggcacttccttcccaggggtctccgccgtccccttccctctccccaccccgggacggtgcaggcaccagtgttccgtgcacctatttatatttttgaaaactaaagattatgataattataataataaagacattggaagagatctaaaaaaaaaaaaaaaaaaaa

SEQ ID NO: 2 - protein, 2132 aa, Mus musculus
MTTLLLVFVTLRVIAAVISEEVPDHDNSLSVSIPQPSPLKVLLGSSLTIPCYFIDPMHPVTTAPSTAPLTPRIKWSRVSKEKEVVLLVATEGQVRVNSIYQDKVSLPNYPAIPSDATLEIQNLRSNDSGIYRCEVMHGIEDSEATLEVIVKGIVFHYRAISTRYTLDFDRAQRACLQNSAIIATPEQLQAAYEDGFHQCDAGWLADQTVRYPIHTPREGCYGDKDEFPGVRTYGIRDTNETYDVYCFAEEMEGEVFYATSPEKFTFQEAANECRRLGARLATTGQLYLAWQGGMDMCSAGWLADRSVRYPISKARPNCGGNLLGVRTVYLHANQTGYPDPSSRYDAICYTGEDFVDIPENFFGVGGEDDITIQTVTWPDLELPLPRNVTEGEALGSVILTAKPIFDLSPTISEPGEALTLAPEVGSTAFPEAEERTGEATRPWGFPAEVTRGPDSATAFASEDLVVRVTISPGAAEVPGQPHLPGGVVFHYRPGSTRYSLTFEEAQQACMHTGAIIASPEQLQAAYEAGYEQCDAGWLQDQTVRYPIVSPRTPCVGDKDSSPGVRTYRVRPSSETYDVYCYVDKLEGEVFFATRLEQFTFQEARAFCAAQNATLASTGQLYAAWSQGLDKCYAGWLADGTLRYPIITPRPACGGDKPGVRTVYLYPNQTGLPDPLSKHHAFCFRGVSVAPSPGEEEGSTPTSPSDIEDWIVTQVGPGVDAVPLEPKTTEVPYFTTEPRKQTEWEPAYTPVGTSPQPGIPPTWLPTLPAAEEHTESPSASEEPSASAVPSTSEEPYTSSFAVPSMTELPGSGEASGAPDLSGDFTGSGDASGRLDSSGQPSGGIESGLPSGDLDSSGLSPTVSSGLPVESGSASGDGEVPWSHTPTVGRLPSGGESPEGSASASGTGDLSGLPSGGEITETSTSGAEETSGLPSGGDGLETSTSGVDDVSGIPTGREGLETSASGVEDLSGLPSGEEGSETSTSGIEDISVLPTGGESLETSASGVGDLSGLPSGGESLETSASGAEDVTQLPTERGGLETSASGVEDITVLPTGRESLETSASGVEDVSGLPSGREGLETSASGIEDISVFPTEAEGLDTSASGGYVSGIPSGGDGTETSASGVEDVSGLPSGGEGLETSASGVEDLGPSTRDSLETSASGVDVTGFPSGRGDPETSVSGVGDDFSGLPSGKEGLETSASGAEDLSGLPSGKEDLVGSASGALDFGKLPPGTLGSGQTPEVNGFPSGFSGEYSGADIGSGPSSGLPDFSGLPSGFPTVSLVDSTLVEVITATTSSELEGRGTIGISGSGEVSGLPLGELDSSADISGLPSGTELSGQASGSPDSSGETSGFFDVSGQPFGSSGVSEETSGIPEISGQPSGTPDTTATSGVTELNELSSGQPDVSGDGSGILFGSGQSSGITSVSGETSGISDLSGQPSGFPVFSGTATRTPDLASGTISGSGESSGITFVDTSFVEVTPTTFREEEGLGSVELSGFPSGETELSGTSGTVDVSEQSSGAIDSSGLTSPTPEFSGLPSGVAEVSGEFSGVETGSSLPSGAFDGSGLVSGFPTVSLVDRTLVESITQAPTAQEAGEGPSGILEFSGAHSGTPDISGELSGSLDLSTLQSGQMETSTETPSSPYFSGDFSSTTDVSGESIAATTGSGESSGLPEVTLNTSELVEGVTEPTVSQELGHGPSMTYISRLSEASGDASASGDLGGAVTNFPGSGVEASVPEASSDLSAYPEAGVGVSAAPEASSKLSEFPDLHGITSAFHETDLEMTTPSTEVNSNPWTFQEGTREGSAAPEVSGESSTTSDIDTGTSGVPSATPMASGDRTEISGEWSDHTSEVNVAISSTITESEWAQPTRYPTETLQEIESPNPSYSGEETQTAETTMSLTDAPTLSSSEGSGETESTVADQEQCEEGWTKFQGHCYRHFPDRETWVDAERRCREQQSHLSSIVTPEEQEFVNKNAQDYQWIGLNDRTIEGDFRWSDGHSLQFEKWRPNQPDNFFATGEDCVVMIWHERGEWNDVPCNYQLPFTCKKGTVACGDPPVVEHARTLGQKKDRYEISSLVRYQCTEGFVQRHVPTIRCQPSGHWEEPRITCTDPNTYKHRLQKRTMRPTRRSRPSMAH

SEQ ID NO: 3 - DNA, 2291 bp, Mus musculus
  CDS (257)..(1363)
aagaggtaatccagaactcaaggagcgcgggcgcagcagctgtgagcattaggtgctgtggccaagcgctgggacaaggatctgccgctaccagcgcatcctctctcctctccagctctgtcccgcactcgctgcacccttccccaggccaccgcgcttcctatgtgatcttccggggcaacgcggagccctttctcacagctcagcagtgaatttccaccccgaaaactgcagtaagccgcctttcaaggacaagatgttgataaatatgaaaggcatcttatggatgtgctcaaccttattactaacccatgcactacatcaagccaaaatggaaaccagcccacctgttaaaggctctctgtctggaaaagtggtcctaccttgtcatctttcaaccttacctaccttaccacccaattacaacacgagtgaatttctcagaatcaaatggtctaagatggaagtggacaaaaatggaaaagatataaaggagacgactgtcttggtggcccagaacggaaatatcaagattggtcaggactacaaggggcgagtgtccgtgcctacacatcccgatgatgtaggtgatgcttccctcaccatggtcaaactccgggctagtgatgcaggcgtctaccgatgtgatgtcatgtatgggattgaagacactcaggacaccatgtcactggctgtggatggtgttgtgtttcactacagggcagccaccagcaggtacactctgaactttgccgctgctcaacaggcttgtttggatatcggggcggtcatagcaagcccagagcagctgtttgccgcctatgaggatggatttgagcagtgtgatgcaggatggctgtctgatcaaactgtcagatatcccatacgggctccccgagagggctgttacggagacatgatggggaaggaaggggttcggacctatggattccgctctccccaggaaacctatgatgtgtattgttatgtggatcatctggatggcgatgtgttccacatcactgctcccagtaagttcaccttcgaggaggccgaagcagagtgtacaagcagggatgcgaggctggcgactgttggagaacttcaggcagcttggagaaatggctttggccaatgcgattacggctggctgtcggatgccagcgtgcggcaccctgtgactgtggccagggcccagtgtggaggaggtctacttggggtgagaaccctgtatcgttttgagaaccagacatgcttccctctccctgatagcagatttgatgcctactgctttaaacgtaagtgtttgatatctcttttaaaaatacagattaaaaagctgcagcaaatatatacccactgaggttccagtggatgtcgggatggattctgtattctgtactaagtgcatggaggacctacagtgcagtgacacaaacagacttaataccaccattatggttaacacagtagggagtttggcaagtgggattttttttgggaggtggggagataacaacgtcagagtggggtcttaactgcaccccagaattccaaaaatcacagaacacaccctgaattgaaatttcttattaattgaagcctttcaatgcaatttcactcttaatataacaatgttaagacaacagcagctgagttaaacctttgaatcctgttttattttttgatatgattcttattataaaaaggagacaatttaagcttaagtcattgcaggtgagtatttgtgctgaaattaataatgattgtgttcaattcctacctaaaaatgcatcatactatcaaataatacgattcacatatctcattttgtttcaagtaatagaataggcgccttttggcgattaatcttgctttgaattaattaaagtacaatacagcctccttgcatgaattggctatgctcagttagatttgccttgtaaaattaattttaggtagcattcattacatatatatatatgtatatatatatattagacattcttaaattattttaaaatattagtcacatacagttttatagttgtagagtgcttaactaaactaataactagtcaatattttattgtaatatgtactttaggatcggtaagatgttagctaacaaatgcctccatactttagcaacagaggccaagtctgacatcgagtatcagaacaatgaaaagccaatgtaacacatgaagtcttttaaaccagctttctaattccttttgttagatcaatgggtaattaaaatagcttgaaaatgtaaaaaaaaaaaaaaa

SEQ ID NO: 4 - protein, 368 aa, Mus musculus
MLINMKGILWMCSTLLLTHALHQAKMETSPPVKGSLSGKVVLPCHLSTLPTLPPNYNTSEFLRIKWSKMEVDKNGKDIKETTVLVAQNGNIKIGQDYKGRVSVPTHPDDVGDASLTMVKLRASDAGVYRCDVMYGIEDTQDTMSLAVDGVVFHYRAATSRYTLNFAAAQQACLDIGAVIASPEQLFAAYEDGFEQCDAGWLSDQTVRYPIRAPREGCYGDMMGKEGVRTYGFRSPQETYDVYCYVDHLDGDVFHITAPSKFTFEEAEAECTSRDARLATVGELQAAWRNGFGQCDYGWLSDASVRHPVTVARAQCGGGLLGVRTLYRFENQTCFPLPDSRFDAYCFKRKCLISLLKIQIKKLQQIYTH

SEQ ID NO: 5 - DNA, 2583 bp, Mus musculus
  CDS (162)..(2339)
cggcagtggctggcaagaaacaattctgcaaaaataatcatacccagcctggcaattgtctgctcctcggtccattgctccgccgccgtccacagtcgcttgcaaggggaaggcactgaatttaccgcggccagaacatccctcccagccggcagtttacaatgctgcgaactaaggatctcatctggactttgtttttcctgggaactgcagtttccctgcaggtagatattgttcccagccaaggagaaatcagcgttggagagtccaaattcttcctgtgtcaagtggcaggagatgccaaagataaggacatctcctggttctcccccaatggggagaagctgagcccaaaccagcagcggatctcagtggtgtggaatgatgacgactcctctaccctcaccatctacaacgccaacatcgacgatgccggcatatacaagtgtgtggtcaccgctgaggacggcacccagtctgaggccactgtcaacgtgaagatcttccagaagctcatgttcaagaatgcaccaaccccacaggagtttaaggaaggggaggatgctgtgattgtctgtgatgtggtcagctccctgcctccaaccatcatctggaaacacaaaggccgagatgtcattctgaaaaaagacgtccggttcatagtcctgtccaacaactacctgcagatcaggggcatcaagaaaacagatgagggtacttaccgctgtgagggcaggatcctggcccgcggggaaatcaacttcaaggacattcaggtcattgtgaatgtaccacccactgtccaggccagacagagcatcgtgaatgccactgccaacctgggccagtctgtcaccctggtgtgtgatgctgatggatttccagagcccaccatgagctggacaaaggatggggaacccatagagaacgaggaggaagatgagagaagcagatcttcagtgagtgacagctccgaggtgaccatcaggaatgtggataaaaacgacgaggccgaatacgtctgcatcgcagagaacaaggctggcgagcaggatgcctccatccacctcaaggtctttgcaaagcccaaaatcacctatgtagagaatcagacggccatggaactagaggagcaagtcactctgacctgtgaagcctccggagaccccatcccgtccatcacgtggagaacatccacccggaacatcagcagtgaagaacaggatctggatgggcacatggtggttcgcagccatgctcgtgtgtcctccttgaccctgaagagcatccagtacagagatgctggagaatacatgtgcactgccagcaacaccatcggccaggactcccagtccatcgacctcgaatttcaatatgctcccaagctccagggccctgtggctgtctacacctgggaagggaaccaagtgaacatcacctgtgaggtctttgcctatcccagtgccacaatctcctggttccgagatggtcagttgctgccaagctccaactacagcaatatcaagatctacaacaccccatctgctagctatctggaggtgaccccagattcagaaaatgactttggaaactacaactgtacagcagtgaaccgtattggacaggagtccttggaattcatccttgttcaagcagacacaccgtcttctccatccattgaccgggtggaaccatactccagcacagcgcaggtgcagtttgatgagccagaggccacagggggcgtgcccattctcaagtacaaggctgagtggaagtcgctgggcgaagaatcctggcatttcacgtggtatgatgccaaagaagccaacatggagggcattgtcaccatcatgggcctgaaacctgagacgacgtactcggatcgactggcggccctcaacggcaaggggctgggcgagatcatgcagccatctgagtccaagacacagccagttccggaactcagtgcacccaagctggaagggcagatgggagaggacgggaactccatcaaggtgaacctgatcaagcaggatgacggaggctcccccatcagacactatctggtcaagtacagagcgctcgcctctgagtggaaaccggaaatcaggctcccatccggcagtcaccacgtcatgctcaagtccctggactggaacgcagagtatgaagtctatgtggtagctgaaaaccagcaaggaaaatccaaggcagctcactttgtgttcaggacctcagcccagcccacggccatcccagccaccctgggcggaagctccacctcctacaccttggtctcattgcttttctctgcggtgactcttcttctgctctgatagaaacttgaacatgcatgtgtgcgagctctcatgtgcgcatgcgcacacgcacacacacgcacacgcacacgcacacgcacgcacacgcacacacacacacacacagacacacacacgcacacacacacgcacatgcacacgcgcacacacgcacacgcacacgcacacgcacgcacacacacacacacacacaccattgtatttgattaaaaagcccagttcctataaaaaaaaaaaaaaa

SEQ ID NO: 6 - protein, 725 aa, Mus musculus
MLRTKDLIWTLFFLGTAVSLQVDIVPSQGEISVGESKFFLCQVAGDAKDKDISWFSPNGEKLSPNQQRISVVWNDDDSSTLTIYNANIDDAGIYKCVVTAEDGTQSEATVNVKIFQKLMFKNAPTPQEFKEGEDAVIVCDVVSSLPPTIIWKHKGRDVILKKDVRFIVLSNNYLQIRGIKKTDEGTYRCEGRILARGEINFKDIQVIVNVPPTVQARQSIVNATANLGQSVTLVCDADGFPEPTMSWTKDGEPIENEEEDERSRSSVSDSSEVTIRNVDKNDEAEYVCIAENKAGEQDASIHLKVFAKPKITYVENQTAMELEEQVTLTCEASGDPIPSITWRTSTRNISSEEQDLDGHMVVRSHARVSSLTLKSIQYRDAGEYMCTASNTIGQDSQSIDLEFQYAPKLQGPVAVYTWEGNQVNITCEVFAYPSATISWFRDGQLLPSSNYSNIKIYNTPSASYLEVTPDSENDFGNYNCTAVNRIGQESLEFILVQADTPSSPSIDRVEPYSSTAQVQFDEPEATGGVPILKYKAEWKSLGEESWHFTWYDAKEANMEGIVTIMGLKPETTYSDRLAALNGKGLGEIMQPSESKTQPVPELSAPKLEGQMGEDGNSIKVNLIKQDDGGSPIRHYLVKYRALASEWKPEIRLPSGSHHVMLKSLDWNAEYEVYVVAENQQGKSKAAHFVFRTSAQPTAIPATLGGSSTSYTLVSLLFSAVTLLLL

SEQ ID NO: 7 - DNA, 3153 bp, Mus musculus
  CDS (151)..(2802)
  misc_feature (3043)..(3043): n is a, c, g, or t
gaggctcccggcgagctggcgcccctgtctgggtcccgcgcgcccggccctgctcgcgcccgcgcatcgcgccgcagtctcggtctgcggctgcgggacgtgacggcgtgcgcggaggggacctcgcaagttcttccatcagtgtgcagaatgataccactgcttctgtccctgctggccgctctggtcctgacccaagcccctgccgccctcgctgatgacctgaaagaagacagctcggaggatcgagccttccgcgtgcgcatcggtgccgcgcagctgcggggcgtgctgggcggtgccctggccatcccatgccacgtccaccacctgcggccgccgcgcagccgccgggccgcgccgggttttccccgggtcaagtggaccttcctgtccggggaccgggaggtagaggttctggtggctcgcgggctgcgcgtcaaggtaaacgaagcctaccggttccgcgtggcgctgcctgcctaccccgcatcgctcacggatgtgtctctagtattgagcgaactgcggcccaatgattccggggtctatcgctgcgaggtccagcacggtatcgacgacagcagtgatgctgtggaggtcaaggtcaaaggggtcgtcttcctctacagagagggctctgcgcgctatgctttctccttcgctggagcccaggaagcctgcgctcgcataggagcccgaatcgccaccccggagcagctctatgctgcctacctcggcggctatgagcagtgtgatgcaggctggctgtccgaccaaactgtgaggtaccccatccagaacccacgagaggcctgctctggagacatggatggctatcctggcgtgcggaactacggagtggtgggtcctgatgatctctatgatgtctactgttatgccgaagacctaaatggagaactgttcctaggcgcccctcccagcaagctgacatgggaggaggctcgggactactgtctggaacgtggtgcacagatcgctagcacaggccagctgtacgcagcctggaatggtggcctggacagatgtagccctggctggctggctgatggcagcgtgcgctatcccatcatcacacccagccaacgctgtgggggcggcctgccaggagtcaagaccctcttcctctttcccaaccagactggcttccccagcaagcagaaccgcttcaatgtctactgcttccgagactctgcccatccctctgcttcctctgaggcctctagcccagcctcagatggacttgaggccattgtcacagtgacagaaaagctggaggaactgcagctgcctcaggaagcgatggagagcgagtctcgtggggccatctactccatccccatctcagaagatgggggaggaggaagctccaccccagaagacccagcagaggcccccaggactccgctagaatcggaaacccaatccattgcaccacctaccgagtcctcagaagaggaaggcgtagccctggaggaagaagaaagattcaaagacttggaggctctggaggaagagaaggagcaggaggacctgtgggtgtggcccagagagctcagcagccctctccctactggctcagaaacagagcattcactctcccaggtgtccccaccagcccaggcagttctacagctggatgcgtcaccttctcctgggcctccaaggttccgtggaccgcctgcagagactttgctccccccgagggagtggagcgccacatctactcctggtggggcaagagaagtagggggggaaactgggagccctgagctctctggggttcctcgagagagcgaggaggcagggagctccagcttggaggatggcccttccctacttccagctacatgggcccctgtgggtcccagggagctggagaccccctcagaagagaagtctggaagaactgtcctggcaggcacctcagtgcaggcccagccagtgctgcccaccgacagtgccagccacggtggagtggctgtggctccctcatcaggtgactgtatccccagcccctgccacaatggtgggacatgcttggaggagaaggagggtttccgctgcctatgtttgccaggctatgggggggacctgtgcgatgttggccttcatttctgcagccctggctgggaggccttccagggagcctgctacaagcacttttccacacgaaggagttgggaggaggcagaaagtcagtgccgagcgctaggtgctcatctgaccagcatctgcacccctgaggagcaagactttgtcaatgatcgataccgggagtaccagtggattgggctcaatgacaggaccatcgagggtgacttcttgtggtcagatggtgcccctctgctctatgaaaactggaaccctgggcagcctgacagctacttcctgtctggggagaactgtgtggtcatggtgtggcatgaccagggacagtggagtgatgtgccctgcaactaccatctatcctacacctgcaagatggggcttgtgtcctgtgggcctccaccacagctacccctggctcaaatatttggtcgccctcggctgcgctacgcggtggatactgtgcttcgatatcgatgccgagacgggctggctcagcgcaacctgccgttgatccgctgccaggagaatgggctttgggaggcccctcagatttcctgtgtaccccggaggcctggccgtgctctgcgctccatggacgccccagaaggaccacggggacagctctcgaggcacaggaaggcaccgttgacaccgccctccagtctctagggagcctggaagactgctgcccccagcaggaccctctcacatcaactgccagtgctcttccccatgatagggggtgacgtgagaggggtgggactgaaattcagaggacagcgctcgaaggggtttctgggaaacacttgggtggctccgccccctcacacaagggcctcaggttttacccggtaagtccctaagtgcctcaactgccctctcatgtcagctgcctccttgtccctcgatntcgtnaggggacactgtgctattcgatcttgattgtcgaagagtttttaggatggagtaccagcaaaaccaggtggaaataaagttgtctgaacccaaagaaaaaaaaaa

SEQ ID NO: 8 - protein, 883 aa, Mus musculus
MIPLLLSLLAALVLTQAPAALADDLKEDSSEDRAFRVRIGAAQLRGVLGGALAIPCHVHHLRPPRSRRAAPGFPRVKWTFLSGDREVEVLVARGLRVKVNEAYRFRVALPAYPASLTDVSLVLSELRPNDSGVYRCEVQHGIDDSSDAVEVKVKGVVFLYREGSARYAFSFAGAQEACARIGARIATPEQLYAAYLGGYEQCDAGWLSDQTVRYPIQNPREACSGDMDGYPGVRNYGVVGPDDLYDVYCYAEDLNGELFLGAPPSKLTWEEARDYCLERGAQIASTGQLYAAWNGGLDRCSPGWLADGSVRYPIITPSQRCGGGLPGVKTLFLFPNQTGFPSKQNRFNVYCFRDSAHPSASSEASSPASDGLEAIVTVTEKLEELQLPQEAMESESRGAIYSIPISEDGGGGSSTPEDPAEAPRTPLESETQSIAPPTESSEEEGVALEEEERFKDLEALEEEKEQEDLWVWPRELSSPLPTGSETEHSLSQVSPPAQAVLQLDASPSPGPPRFRGPPAETLLPPREWSATSTPGGAREVGGETGSPELSGVPRESEEAGSSSLEDGPSLLPATWAPVGPRELETPSEEKSGRTVLAGTSVQAQPVLPTDSASHGGVAVAPSSGDCIPSPCHNGGTCLEEKEGFRCLCLPGYGGDLCDVGLHFCSPGWEAFQGACYKHFSTRRSWEEAESQCRALGAHLTSICTPEEQDFVNDRYREYQWIGLNDRTIEGDFLWSDGAPLLYENWNPGQPDSYFLSGENCVVMVWHDQGQWSDVPCNYHLSYTCKMGLVSCGPPPQLPLAQIFGRPRLRYAVDTVLRYRCRDGLAQRNLPLIRCQENGLWEAPQISCVPRRPGRALRSMDAPEGPRGQLSRHRKAPLTPPSSL

SEQ ID NO: 9 - DNA, 3977 bp, Mus musculus
  CDS (232)..(2784)
gaattcgcggccgcgtcgaccggatggcgcagttgaactgcgctgctgagctcgcggccgcctgcgcgcgctggggggactcgtctgggctactcccccacctcgcgttggacgaccggtcctggacacaccgcttgcgaggcaagttgaacattgcagagaaggatcttaaggctacacccgacttgccacacttgccatccagctgaagaaccaaaggctgttggagagatggcagtgacatcccaccacatggtccctgtgtttgtcctgatgagcgcctgcctggccaccgcaggtccagagcccagcacccggtgtgaactgtcaccgatcagtgcctctcatccagtccaggccctgatggagagcttcaccgttctgtctggctgtgccagcagaggcaccactgggctgccaagggaggttcacatcctaaacctccgcagtacagaccaaggactaggccagccgcagagagaggttaccctgcatctgaaccccattgcctccgtgcacactcaccacaagcctgttgtgttcctgctcaactccccacagcccctggtgtggcatgtgaagacagagagactggctgctggtgtccccagactcttcctggtttcggagggttctgtggtccagttttcatcaggaaacttctccttgacagcagaaacagaggaaaggagtttccctcaagaaaatgagcatctgctacactgggcccaaaaggaatatggagcagtgacttcattcaccgaacttaagatagcaagaaatatctatattaaagtgggagaagatcaagtgttccctcccacgtgtaacatagggaaaaattttctctcgctcaattaccttgcggagtaccttcaacccaaagccgccgaaggttgtgtcctggccagtcagccccacgagaaggaagtgcatatcattgagttaatctcccccaactccaatccttacagcaccttccaggtggatataataattgacatacgacctgctcgagaggatcctgaggtggtcaaaaacctcgtcctgatcttgaagtgcaaaaaatctgtcaactgggtgatcaagtcttttgacgtcaagggaaacttgaaagttattgctcctgacagtattggctttggaaaagagagtgaacgatccatgacagtgaccaaattggtaagaaatgactacccttccacccaagagaatctgatgaagtgggcattggacaatggctacagcccagtgacgtcatacaccatagctcctgtggccaatagatttcatcttcggcttgagaacaacgaggagatgagagatgaggaagtccacaccattcctcctgagcttcggatcctgctgggccctgaccacctgcctgccctggacagcccacccttccaaggggaaatcccaaatggaggtttcccctttccattcccggatatccccaggagaggctggaaggagggagaagataggatcccccggccaaaggaacccatcattcccagagttcaattgcttccagaccacagagagccagaagaagtgcaagggggcgtgaatatcgccctgtcagtcaaatgtgacaatgaaaagatggtggtagctgtagacaaagattctttccagaccaatggctactcggggatggagctcaccctgttggatccttcctgcaaagccaagatgaatggtacccactttgttctggagtctccgctgaatggctgtggtactagacatcggaggtcagccccagatggtgtggtttactataactctattgtggtgcaggctccatcccctggggatagcagtggctggccagacggctacgaagatttggagtcgggtgataatggatttcctggagacacagatgaaggagaaactgcccccctgagccgtgctggagtggtagtgtttaactgcagcttgcggcagctgaggagtcccagtggcttccaggaccagctcgatggaaatgctaccttcaatatggagctgtataacacagacctctttctggtgccctccccgggggtcttctctgtggcagagaatgagcatgtatatgttgaggtgtctgtcactaaggctgaccaagatctgggatttgccatccaaacctgctttatctctccatactcaaacccagacagaatgtctgattacaccatcatcgagaacatctgtccgaaggatgactctgtgaagttctacagctccaagagagtgcacttccccatcccacatgctgaagtggacaagaagcggttcagctttgtgttcaagtccgtgttcaacacctccctgctcttcctgcactgcgagctgacgctgtgctctaggaacaagggctcccagaagctgccaaagtgtgtgactcctgatgacgcctgcacctctctagatgccaccatgatctggaccatgatgcagaataagaagacattcaccaagcccctggccgtggtcctccaagtagattataaagaaaatgttccaaacatgaaggagtccagtccggttcctcctcctccacagattttccacggcctggacacgctcaccgtgatgggcattgcgtttgcagcatttgtgatcggagcactcctgacgggggccttgtggtatatctactcccacacaggggagacggcacgaaggcagcaagtccctacctcgccaccagcctcggagaacagcagcgcagcccacagcatagggagcactcagagcactccctgctctagcagcagcacggcctaggtggacagatggacgccgctgcctaggtggacagacggatgcctgccaccgccggggcagggccagatgccgatgctgggtgtccagactcagaaggcttgatctgggttccctgtaaagagagagtgaatttcagtatacagacagcctgctctacccaccccttccttaccacggcccacgtaaatgtgaccctggacatctgtcacacgaaagctaagctggtggccttccctgccagcccctcgcaggatgggggtttcaatgtgaaacatatgccagtttttgttctttttttttttttattttttatgctgctttgtccaggtgtccaaacatccatcatttggggtcgtatgttttaaagagtaaaggagacggtgaagggatgtcagctagagcgtagagccagggagagacagccagggattctcgcctagctgaaccaagatgtaaaataggagaaatgctccctccatagccaatctgcatctcgctctggatgcccactcctgacctgccacgccctacatggtcccctgtgtagcttgctagccttgggggcctgtcacttgccaaagtaacattcctctcttgggccaaaatgaatgctgactcctgatactccgcacaagcacaccaactccaaaagtgccttaaagccaggtttgtgtctcctttcaggagagagcgtgtaacgtcagatttgtgcctgtctcgaacattctgtcctaatcatacttaaacatcaggcaggtggggttcagtggtaagcgaagggattattagcaaggtaatgcctgtgtatggtcatataggcacaaatgcagaacacccagcacattggagagacgaccacttcctcctttaaaatgtcattgcttccagagtcggaacctgagttatatgtggtagattatattgactccaaccatcagcctttctgatgagcctttagagaagcctagagagcccgggcatctgatgatgtcacagtgctgcctcagatgatacagagacagggagtttcaggtgaacttcttcagcagaataaggtgatgccactctgacctttgctgagctcagtgactcttcagtgttaaaactgaacccagaggggattctccatgccagatccgctgcaaattaaataataccaattttctagagtccgtcgacgcggccgcgaattc

SEQ ID NO: 10 - protein, 850 aa, Mus musculus
MAVTSHHMVPVFVLMSACLATAGPEPSTRCELSPISASHPVQALMESFTVLSGCASRGTTGLPREVHILNLRSTDQGLGQPQREVTLHLNPIASVHTHHKPVVFLLNSPQPLVWHVKTERLAAGVPRLFLVSEGSVVQFSSGNFSLTAETEERSFPQENEHLLHWAQKEYGAVTSFTELKIARNIYIKVGEDQVFPPTCNIGKNFLSLNYLAEYLQPKAAEGCVLASQPHEKEVHIIELISPNSNPYSTFQVDIIIDIRPAREDPEVVKNLVLILKCKKSVNWVIKSFDVKGNLKVIAPDSIGFGKESERSMTVTKLVRNDYPSTQENLMKWALDNGYSPVTSYTIAPVANRFHLRLENNEEMRDEEVHTIPPELRILLGPDHLPALDSPPFQGEIPNGGFPFPFPDIPRRGWKEGEDRIPRPKEPIIPRVQLLPDHREPEEVQGGVNIALSVKCDNEKMVVAVDKDSFQTNGYSGMELTLLDPSCKAKMNGTHFVLESPLNGCGTRHRRSAPDGVVYYNSIVVQAPSPGDSSGWPDGYEDLESGDNGFPGDTDEGETAPLSRAGVVVFNCSLRQLRSPSGFQDQLDGNATFNMELYNTDLFLVPSPGVFSVAENEHVYVEVSVTKADQDLGFAIQTCFISPYSNPDRMSDYTIIENICPKDDSVKFYSSKRVHFPIPHAEVDKKRFSFVFKSVFNTSLLFLHCELTLCSRNKGSQKLPKCVTPDDACTSLDATMIWTMMQNKKTFTKPLAVVLQVDYKENVPNMKESSPVPPPPQIFHGLDTLTVMGIAFAAFVIGALLTGALWYIYSHTGETARRQQVPTSPPASENSSAAHSIGSTQSTPCSSSSTA

SEQ ID NO: 11 - DNA, 1767 bp, Mus musculus
  CDS (207)..(1271)
gactaatgttggggtaaacacagaaagcccattataacatgataagttcattaaaagaggtcagaatatcgtgcttcagatcacagaagcggtaacgagcagcaccctgctcagaggagaagtgaggggagacaggggcaggagacccagaatctcacgtaacctttaaacttcttcagaaggtcgtgaaaatacatgagataatcatgaaggcaactctcatcttcttccttctggcacaagtctcttgggctggaccatttgaacagagaggcttatttgacttcatgctagaagatgaggcttctggcataatcccttatgaccctgacaatcccctgatatctatgtgcccctaccgatgccagtgtcatcttcgagtggtgcagtgttctgatctgggtttggacaaagtgccctgggattttccacccgacacaaccttgctagacctgcaaaacaacaaaattacagagatcaaagaaggggccttcaagaacctgaaggacttgcataccttgatccttgtcaacaacaagatcagcaaaatcagtccagaggcattcaaacctctcgtgaagttggaaaggctttacctgtctaagaaccaactaaaggaactgcctgaaaaaatgcccagaactctccaggaacttcgtgtccatgagaatgagatcaccaagctgcggaaatccgacttcaatggactgaacaatgtgcttgtcatagaactgggcggcaacccactgaaaaactctgggattgaaaacggagccttccagggactgaagagtctctcatacattcgcatctcagacaccaacataactgcgatccctcaaggtctgcctacttctctcactgaagtgcatctagatggcaacaagatcaccaaggttgatgcacccagcctgaaaggactgattaatttgtctaaactgggattgagcttcaacagcatcaccgttatggagaatggcagtctggccaatgttcctcatctgagggaactccacttggacaacaacaaactcctcagggtgcctgctgggctggcacagcataagtatatccaggtcgtctaccttcacaacaacaacatctccgcagttgggcaaaatgacttctgccgagctggacacccctctcgaaaggcttcctactcggctgtgagtctttacggcaaccctgtccggtattgggaaatctttccaaacaccttcagatgtgtctatgtgcgttctgccattcaacttggaaactacaagtaaccctcagacggcctaattcttataatctggaaaaacaccccaatatgtcaatatcattgctaaaaagaaaaaatactaaaaaagaaagaaagaatgctagattctgaaaattcaagcacactgtgcatgccttttccacatgacttattatgcaagctgaatatacagattgaataattgcctacaataaaaatattttacttgtagtattcagaatcacttttaaagttgtctgtagttgtgaactgagttatcaaagtctgatgtaatcataaatgtcaaccacttagtaaagcgttaaaaggaacagaatacaaagtctctgtaattcatagagctaaattaactcttttgacataaagtcaaatgccgccgaactctagcaatgtattaatctcctttattattggtgaagccttatttgagtatgtaagttcaatatgggctatgtaaaattttcactgtcatattgaaaaaaataaaattttaaaaatgc

SEQ ID NO: 12 - protein, 354 aa, Mus musculus
MKATLIFFLLAQVSWAGPFEQRGLFDFMLEDEASGIIPYDPDNPLISMCPYRCQCHLRVVQCSDLGLDKVPWDFPPDTTLLDLQNNKITEIKEGAFKNLKDLHTLILVNNKISKISPEAFKPLVKLERLYLSKNQLKELPEKMPRTLQELRVHENEITKLRKSDFNGLNNVLVIELGGNPLKNSGIENGAFQGLKSLSYIRISDTNITAIPQGLPTSLTEVHLDGNKITKVDAPSLKGLINLSKLGLSFNSITVMENGSLANVPHLRELHLDNNKLLRVPAGLAQHKYIQVVYLHNNNISAVGQNDFCRAGHPSRKASYSAVSLYGNPVRYWEIFPNTFRCVYVRSAIQLGNYK

SEQ ID NO: 13 - DNA, 1474 bp, Mus musculus
  CDS (152)..(556)
gcctctgtctccctctctccacgaactgcccaggagcgagcagctgctcccggttggccctgacggacagacaaaccgacagcctgacaacctagtccaccaactaagcagcctgcacctggctgcttgtccctccccaggaacattgaccatgtgtcccctgtggctactcaccttgctgctggccctgagccaggccttgccctttgagcagaagggtttctgggacttcaccttggatgatgggctgctcatgatgaatgatgaggaggcttcaggttcagacaccacttcaggtgtccccgacctggactctgtcacacctaccttcagtgccatgtgtcctttcggttgccactgccacctgcgggttgttcagtgctctgacttgggtctgaagactgtgcccaaggagatctcacctgacaccacactgctagacctgcagaacaatgacatttctgagcttcgcaaggatgacttcaaaggcctccagcacctctacgtaagcctcctcctgcctcctgccttttctgaggagccaggaggagtttgatgttcctatgacttggaagaagggtactcacaaatgaggttgggaagaggggtgggaaccgatggagtcctgagataagtctggagctggctgtaatgagaacgcccaaaaccatgcattatatgtatgggtacaagacgaaatctgtcaggtcccatttttttttgacagaaatctttacgttttgttgactcatctgaacaacacagaaagtgtattccatcatagctcttagagaaactgtctagcaaagtatgctaggcagtgatggcattaccagggcagcttggagctaacctcagctccttgccctacactctactcaccaacacatactgatctccatgagaccattaaaggttcagatgtttcccaagtgccatcagtagcaagttcaggaggcagaggagaggggaggtggagtggggggctggggtgctcaccctggttgcctgccccgttttaggccctggtcttggtaaacaataagatctccaagatccatgagaaggcctttagccctctgcggaagctgcaaaaactctacatctccaagaaccacctggtggagattcctcccaacctgcccagctccctggtagaactacgaatccatgacaaccgtatccgcaaagtgcccaagggcgtgttcagcgggctccggaacatgaactgcattggtgagactagtctgtaccttagcatcaggcataaaggacacaggtagaggatatctctttcagccagggcagctgggtccatgtagtcatggaaaagccctgtgtgcctcagaatgttccagatgtttgttttgggacatgaagaaaaagactgtggtggctataaaggctcggttgaagaaagagtctcaaatatagtctaaaaaagaccagtatgtttctgaaaaaaaaaaaaaaa

SEQ ID NO: 14 - protein, 134 aa, Mus musculus
MCPLWLLTLLLALSQALPFEQKGFWDFTLDDGLLMMNDEEASGSDTTSGVPDLDSVTPTFSAMCPFGCHCHLRVVQCSDLGLKTVPKEISPDTTLLDLQNNDISELRKDDFKGLQHLYVSLLLPPAFSEEPGGV

SEQ ID NO: 15 - DNA, 2860 bp, Mus musculus
  CDS (78)..(1208)
gctgccacattctccaacccaaggagaccagacagaaggacgtggtcactctgaacagttcaacccaagagacaaaaatgcagtgggcctccgtcctgctgctggctgggctctgctcactttcccagggccagtatgatgaagactctcactggtggatccaatacctccgaaaccagcagtccacctactacgacccctatgacccttacccctatgagccctctgagccttacccctatggagtggaagaaggcccagcctatgcctatggtgcaccacctccaccagagccccgtgattgtccccaagaatgcgactgtccccccaacttccccacagccatgtactgtgacaaccgcaacctcaagtacctgccttttgtgccctcccgcatgaagtacgtctacttccagaacaaccagatctctgccatccaggaaggtgtctttgacaatgccactgggctcctctgggtcgctctacatggcaaccagattaccagtgacaaggtaggcaggaaagtcttctccaagctgaggcacttggagaggctgtacttggaccacaacaacctgacccggatgcccggcccgctgccccgatccctgagagagctccacctggaccacaaccagatctcacgggtccccaacaatgctttggagggcctggagaacctcacggccttatatctccaccacaatgagatccaggaagtggggagttccatgagaggcctccggtccctgatcctactagacctgagttataaccaccttcggagggtacccgacggtctgccctcagccctggagcagctgtacctagaacacaacaatgtctacaccgtccctgacagctacttccgggggtcacccaagctgctgtacgtccggttgtctcacaacagtctcactaacaacggccttgctaccaacaccttcaactccagcagccttcttgagctagacttgtcttacaatcagctgcagaagatccctcctgtcaacaccaacctggagaatctttacctccagggcaacaggatcaatgagttctccatcagcagcttctgcacggtggtggacgtcatgaacttctccaagctgcaggtgctacgcctggatgggaacgagatcaagcgcagcgcaatgccggtggacgccccactctgcctgcgcctcgccaacctcatcgagatctgaggggcctcgccttgggctcctggctccgggctccgagtaccgtgcaccgtgcaccgggctcggggtacatgccctgccaccccgctgcatgtttggcttttgctggatggtctgggacacgcatgtgacagaagtccacaggatcttattcaatctccttccaacaggcagagttaggtgggatcaggggccaggccagtttctgcagggggatgaatttggtggtaaaagaaatatagctatagagtctagccccaaaatcttcttgcttggacagtagcatataaccagttccaatttgaccttttttgagctgtgctcaacagcatggccagctgcttctgcagttgctctgggctcctgctctttgctcctcaaaacatcacaacacacctcttgcccagtcacctcctcccagccccagctcaccctctctgccctctttcactggggcctcttctaatgtcttaggcagtcaggagacacccacaccctaagggcacactcttatctgaggctaaacggatggctaaaatcacacacttacccccacctcacctgtttaaagtcaccatattgaacaccataatgatgagctgttaacataggggataaccgactctataatggaggatctatcagaggagggaagtgtccagtggtcattatcagtatccatagtaagatggggaagagacacacctcaggatagcagaactgaaggggcagccccctttccagtttcatctgacacaacatgatctgcaccccttcttgggcttttagtatcgaaggggcaagcgtggttttcaaaacatgagaaagagcctctgctcatcctttggtctagtatgaagggttgttacgcaaatggcagaggcagactccacccgccgaggacctacccactaacccagactagggggtgatctcaatagctcgtttgtccataatgctagagcatcaggaaccagccctgtggagatgctcactggggtgcaagaagccctgccaggcccaagtcctgccgacactagacctggcttcctctgcattcacactatgcagtccgccttatgcccagtggtgcctgtgatttgtagtttgtctaagaacagccccatcttctcctttttaaaggtgatattgcctaggtacaaccaacagcttcttctagaccccactgggatagagccaccagggtagcgagtggcaagaagcagttgtaagatgctagcctgaggtgggtccctgccttagacccctgactctgtgtgcacttgggagcaagcttctctggaaaggacagggtggggcggggtgcttagactgtgctacgggttccagaactgcaatccacaaaagccaaaccagcttgtttcaaccggggagtgccacctgcggagcaaggctgccctggccagggttcttgggaagcacctgcatgtatcaccatcctgcctctgaagagccctcagcatgaagcaagcatgaatggcggccaacctaaccaataaagttattttataagtgcatctgcaaggatggaatggttggtggcagccctcggcatcgcctcggggtacctagacacacaggccttgaaccgtgtcagctctttataaagagtagcctatataaaaactcaagtt

SEQ ID NO: 16 - protein, 376 aa, Mus musculus
MQWASVLLLAGLCSLSQGQYDEDSHWWIQYLRNQQSTYYDPYDPYPYEPSEPYPYGVEEGPAYAYGAPPPPEPRDCPQECDCPPNFPTAMYCDNRNLKYLPFVPSRMKYVYFQNNQISAIQEGVFDNATGLLWVALHGNQITSDKVGRKVFSKLRHLERLYLDHNNLTRMPGPLPRSLRELHLDHNQISRVPNNALEGLENLTALYLHHNEIQEVGSSMRGLRSLILLDLSYNHLRRVPDGLPSALEQLYLEHNNVYTVPDSYFRGSPKLLYVRLSHNSLTNNGLATNTFNSSSLLELDLSYNQLQKIPPVNTNLENLYLQGNRINEFSISSFCTVVDVMNFSKLQVLRLDGNEIKRSAMPVDAPLCLRLANLIEI

SEQ ID NO: 17 - DNA, 1723 bp, Mus musculus
  CDS (125)..(1093)
gacagttggaagatgacagttgaccagaattcctatccattggtcaggggcaaataccaaggactctgtatgctcagtcactctgtgcgacttcatcaagtcggagccaaagaaatcttgaaagatgggaatgctagcaagagttgccctgggacttatcatcattgatgctgtattggctgcaccaactacagagctctttaactatgactcagaagtgtatgatgccatcttggaggacacagggaccttttataactatgaacacattcctgacaaccatgtagagaacgagaaagtgtctgagaggctttctgggaacagagagctcctgaccccaggcccacagctaggggacaaccaggatgaagacaaggatgaagaatctactcccaggctgatcgatggctcttccccacaggagccagaattcccggggcttctgggtccacacaccaacgaagactttccaacttgtctcctgtgtacttgtataagtaccactgtatactgtgatgaccatgaactggacgctattcctccactgcccaagaagactacatatttctattcacgttttaacagaattaaaaagatcaacaaaaatgactttgcaagcttaaatgatttaaaaaggatcgatctgacatcaaatttaatatcggagattgatgaagatgcattccgaaagctgcctcacctccaagagctggttctgcgtgacaataagataaagcagcttccagaattgccaaacactttgacatttattgatatcagcaacaatagactgggaagaaaaggcattaaacaagaagcttttaaagatatgtatgatctccaccatctctatatcaccgataacagcttggaccacatccctctaccactcccagaaagtctgcgggctcttcacctccagaataatgacattctggagatgcatgaagatactttctgcaatgttaaaaatttaacttatgttcgtaaggcattagaggatattcgactggatggaaaccctatcaacctcagcagaactcctcaagcctacatgtgtctacctcgtttgcccattgggagttttatctaatgttaggatgctaggcaaatatcagaattgtgatgctttctgtagccaagagaagcatatatctcttcagaagcaaactcagtatatttgagatgcgtttaaaacatgtggcacaatgatataaacaataagctaaaggtgttgagataaaataagagttcaataatttaattaaacatgattaggtataaattaacaaaatatatagacagtaagttaaataaaatgtatgccaactagtcatcaaatctcatcaagaattgtaggtgttcatgtcaagttaaatatgtaagaaataaaatattggtaatgaatactttagatacgttgctaagatgtagacattttaattaaacttctctagtctttattttcactcatgtatgtttcccacctgattcagttaagagcagaaaaaaatattaaatgtgtttgaaagatatatggtcttgtgaaaaagtatttcttataattgtagtgtgcaaataactatgtaatagcagtatacattccttcagtttctaatgacatctatcatctataagaacatcaactcattttgcaaacttggttagttgacagctacctgtattcctctacatacaaaaaataaaatatgagatatagagc

SEQ ID NO: 18 - protein, 322 aa, Mus musculus
MGMLARVALGLIIIDAVLAAPTTELFNYDSEVYDAILEDTGTFYNYEHIPDNHVENEKVSERLSGNRELLTPGPQLGDNQDEDKDEESTPRLIDGSSPQEPEFPGLLGPHTNEDFPTCLLCTCISTTVYCDDHELDAIPPLPKKTTYFYSRFNRIKKINKNDFASLNDLKRIDLTSNLISEIDEDAFRKLPHLQELVLRDNKIKQLPELPNTLTFIDISNNRLGRKGIKQEAFKDMYDLHHLYITDNSLDHIPLPLPESLRALHLQNNDILEMHEDTFCNVKNLTYVRKALEDIRLDGNPINLSRTPQAYMCLPRLPIGSFI

SEQ ID NO: 19 - DNA, 4909 bp, Mus musculus
  CDS (390)..(3296)
gcagctccgagctaggtgctatcgcaaggccagagcgcacagcccggcggagagagcagatccttgctcagatcgagtcaaatcggggccaaggcggaggacgaagagtccaggctcctattctggacttgttccccagctccgggggcgcttctaggtcctgcagcagccaggagtgcggagccaccaactcggtgctggaatgaaaaaattcccgcgcgccagtgcagaatctttctaagtgacccggagcttcgggtgctagctctgcacgaactttcccatcaaagtgatcgtgaattttaagcatcaggagcaggccagcgaagctctacgcgtctaaacgtctatccagaccaagagttctctgcggtgcagggtgcggtgccatgcagccaaaagtccctttggggtcacgcaagcagaagccctgctccgacatgggggacgtccagcgggcagcgagatctcggggctctctgtccgcacacatgctgttgctgctcctcgcttccataacaatgctgctatgtgcgcggggcgcacacgggcgccccacggaggaagatgaggagctggtcctgccctcgctggagcgcgccccgggccacgattccaccaccacacgccttcgtctggacgcctttggccagcagctacatctgaagttgcagccggacagcggtttcttggcgcctggcttcaccctgcagactgtggggcgcagtcccgggtccgaggcacaacatctggaccccaccggggacctggctcactgcttctactctggcacggtgaacggtgatcccggctctgccgcagccctcagcctctgtgaaggtgtgcgtggtgccttctacctacaaggagaggagttcttcattcagccagcgcctggagtggccaccgagcgcctggcccctgccgtgcccgaggaggagtcatccgcacggccgcagttccacatcctgaggcgaaggcggcggggcagtggcggcgccaagtgcggcgtcatggacgacgagaccctgccaaccagcgactcgcgacccgagagccagaacacccggaaccagtggcctgtgcgggaccccacgcctcaggacgcgggaaagccatcaggaccaggaagcataaggaagaagcgatttgtgtccagcccccgttatgtggaaaccatgctcgtggctgaccagtccatggccgacttccacggcagcggtctaaagcattaccttctaaccctgttctcggtggcagccaggttttacaagcatcccagcattaggaattcaattagcctggtggtggtgaagatcttggtcatatatgaggagcagaagggaccagaagttacctccaatgcagctctcacccttcggaatttctgcaactggcagaaacaacacaacagccccagtgaccgggatccagagcactatgacactgcaattctgttcaccagacaggatttatgtggctcccacacgtgtgacactctcgggatggcagatgttggaactgtatgtgaccccagcaggagctgctcagtcatagaagatgatggtttgcaagccgccttcaccacagcccacgaattgggccatgtgtttaacatgccgcacgatgatgctaagcactgtgccagcttgaatggtgtgactggcgattctcatctgatggcctcgatgctctccagcttagaccatagccagccctggtcaccttgcagtgcctacatggtcacgtccttcctagataatggacacggggaatgtttgatggacaagccccagaatccaatcaagctcccttctgatcttcccggtaccttgtacgatgccaaccgccagtgtcagtttacattcggagaggaatccaagcactgccctgatgcagccagcacatgtactaccctgtggtgcactggcacctccggtggcttactggtgtgccaaacaaaacacttcccttgggcagatggcaccagctgtggagaagggaagtggtgtgtcagtggcaagtgcgtgaacaagacagacatgaagcattttgctactcctgttcatggaagctggggaccatggggaccgtggggagactgctcaagaacctgtggtggtggagttcaatacacaatgagagaatgtgacaacccagtcccaaagaacggagggaagtactgtgaaggcaaacgagtccgctacaggtcctgtaacatcgaggactgtccagacaataacggaaaaacgttcagagaggagcagtgcgaggcgcacaatgagttttccaaagcttcctttgggaatgagcccactgtagagtggacacccaagtacgccggcgtctcgccaaaggacaggtgcaagctcacctgtgaagccaaaggcattggctactttttcgtcttacagcccaaggttgtagatggcactccctgtagtccagactctacctctgtctgtgtgcaagggcagtgtgtgaaagctggctgtgatcgcatcatagactccaaaaagaagtttgataagtgtggcgtttgtggaggaaacggttccacatgcaagaagatgtcaggaatagtcactagtacaagacctgggtatcatgacattgtcacaattcctgctggagccaccaacattgaagtgaaacatcggaatcaaagggggtccagaaacaatggcagctttctggctattagagccgctgatggtacctatattctgaatggaaacttcactctgtccacactagagcaagacctcacctacaaaggtactgtcttaaggtacagtggttcctcggctgcgctggagagaatccgcagctttagtccactcaaagaacccttaaccatccaggttcttatggtaggccatgctctccgacccaaaattaaattcacctactttatgaagaagaagacagagtcattcaacgccattcccacattttctgagtgggtgattgaagagtggggggagtgctccaagacatgcggctcaggttggcagagaagagtagtgcagtgcagagacattaatggacaccctgcttccgaatgtgcaaaggaagtgaagccagccagtaccagaccttgtgcagaccttccttgcccacactggcaggtgggggattggtcaccatgttccaaaacttgcgggaagggttacaagaagagaaccttgaaatgtgtgtcccacgatgggggcgtgttatcaaatgagagctgtgatcctttgaagaagccaaagcattacattgacttttgcacactgacacagtgcagttaagaggcgttagaggacaaggtagcgtggggagggctgatacactgagtgctggagggatccagtgagtcaaaccagtaagcagtgaggtgtggcaaggaggtgtgtgtaggggatacatagcaaaggaggtagatcaggacactaccctgccagttacattctgataaggtagttaatgaggcacagtagcatctgaaagaccatacagagcactaaggagccccaaagcactattagtatctcttttcttatatctatcgcccaaataattttcagagtctggcagaagccctgttgcactgtactgactagatacttcttatcacaaagattgggaaaggcaaagcagaaagatggtaagactgggtttcaaacaaggcttggtttctatcactggaggcaaggaggaggggacaaacaagatcattattcgaagtcgctggttgctgtggttttacggaaggttgatgcatcattcctatcaacagtgaaaagttcagcttgttcaacgtgacagaaaggctcatctccgtgaaagagctcctgatttcttcttacaccatctcagttcttaactataattcatgttgaggtagaaacaattcatctatttataaaatgtacattggaaaaaaaaaagtgaagtttatgaggtacacataaaaactgaaggaaacaatgagcaacatgcctcctgctttgcttcctcctgaggtaaacctgcctggcgattgaggttgtttaagattatccatggctcacaagaggcagtaaaataatacatgttgtgccagagttagaatggggtatagagatcagggtcccatgagatggggaacatggtgatcactcatctcacatgggaggctgctgcagggtagcaggtccactcctggcagctggtccaacagtcgtatcctggtgaatgtctgttcagctcttctactgagagagaatatgactgtttccatatgtatatgtatatagtaaaatatgttactatgaattgcatgtactttataagtattggtgtgtctgttccttctaagaaggactatagtttataataaatgcctataataacatatttatttttatacatttatttctaatgataaaacctttaagttatatcgcttttgtaaaagtgcatataaaaatagagtatttatacaatatatgttaactagaaataataaaagaacacttttgaatgtgtatgcctattttctggagtgggattaacttctgggcaagaaatctgatgagacacaaacattggacttcaagacagttttaaattttgggtaaatgaactgtatttcctgtttatagacgtactaataaaaaagaagttgatgatgtctttagtggtaagattgttactaatgtggttggcaaattgctgtaaagagccagatagtaagcatttatggcattgtaggctatctttcctgccacaaccatgtgacagtgagtgctttgtaggactgagagcagccataaatgacatgtaaatgataaactgtggctgtgctttaataaaactttatttacaaaaacaaaaaaaaaaaaaaaaaaaaaaaaaaaaaaaaaaaaaaaaaaaaaaaaaa

SEQ ID NO: 20 - protein, 968 aa, Mus musculus
MQPKVPLGSRKQKPCSDMGDVQRAARSRGSLSAHMLLLLLASITMLLCARGAHGRPTEEDEELVLPSLERAPGHDSTTTRLRLDAFGQQLHLKLQPDSGFLAPGFTLQTVGRSPGSEAQHLDPTGDLAHCFYSGTVNGDPGSAAALSLCEGVRGAFYLQGEEFFIQPAPGVATERLAPAVPEEESSARPQFHILRRRRRGSGGAKCGVMDDETLPTSDSRPESQNTRNQWPVRDPTPQDAGKPSGPGSIRKKRFVSSPRYVETMLVADQSMADFHGSGLKHYLLTLFSVAARFYKHPSIRNSISLVVVKILVIYEEQKGPEVTSNAALTLRNFCNWQKQHNSPSDRDPEHYDTAILFTRQDLCGSHTCDTLGMADVGTVCDPSRSCSVIEDDGLQAAFTTAHELGHVFNMPHDDAKHCASLNGVTGDSHLMASMLSSLDHSQPWSPCSAYMVTSFLDNGHGECLMDKPQNPIKLPSDLPGTLYDANRQCQFTFGEESKHCPDAASTCTTLWCTGTSGGLLVCQTKHFPWADGTSCGEGKWCVSGKCVNKTDMKHFATPVHGSWGPWGPWGDCSRTCGGGVQYTMRECDNPVPKNGGKYCEGKRVRYRSCNIEDCPDNNGKTFREEQCEAHNEFSKASFGNEPTVEWTPKYAGVSPKDRCKLTCEAKGIGYFFVLQPKVVDGTPCSPDSTSVCVQGQCVKAGCDRIIDSKKKFDKCGVCGGNGSTCKKMSGIVTSTRPGYHDIVTIPAGATNIEVKHRNQRGSRNNGSFLAIRAADGTYILNGNFTLSTLEQDLTYKGTVLRYSGSSAALERIRSFSPLKEPLTIQVLMVGHALRPKIKFTYFMKKKTESFNAIPTFSEWVIEEWGECSKTCGSGWQRRVVQCRDINGHPASECAKEVKPASTRPCADLPCPHWQVGDWSPCSKTCGKGYKKRTLKCVSHDGGVLSNESCDPLKKPKHYIDFCTLTQCS

SEQ ID NO: 21 - DNA, 3673 bp, Mus musculus
  CDS (392)..(2929)
ggggagaacccggggaagacccacagatacacagaaacgagagagacagaagaggagagagacagagacaaagagacagcagcaggaggaggaggaggaggaggcaaagacagggcgagcactgaggcaaagccagacagctgagcagagggagaagccaaggagtcacagacaaagacctgggacagaaaggcaaaggaggaagttttggagcagaaagaacccgtgggcacttttcctgagcccaggggctgttttctttttcatcttctctgaaagtccctagcccatttaaaaactttgcctcagattttggcacaagccccaaagcccttctggctttaacgaggagccttccacagttagggtgtggagcattttggtgccgcagatggcctcaatccatcccagctgcagcccgggtaccatgtcccagatgggcttgcatcccaggaggggcttgactgggcactggctgcaaagattccaaccctgcttgccgcttcacactgtgcagtggcggaggctgctgctgctggccttcctcctgtccttagcgtggcccgccagccccctcccccgggaggaggagatcgtgtttccagagaagctcaatggcagtagcatcctacctggatcaggcgttcctgccaggctgctgtaccgattgccagcctttggggagatgttgctactagaactagaacaggaccctggggtgcaggtagagggtttgactgtacagtacctgggccaggcacctgagatgctgggtggggcagagccaggtacctacctgactggcaccatcaatggagatccggagtcggtggcatctctgcactgggacgggggagccctattaggggtactgcagtaccgtggggccgaactccacctccagcctctggaaggaggcgcccttaactctgctgggggaccgggggctcacatcctacgccggaagagtcctgccagcagccaaggtcccatgtgcaccgtcaaggctccttctgggagcccgagtcccatttcccgcagaaccaagcgcttcgcttctctgagtagattcgtggagacactggtggtagcagatgacaagatggcagcattccatggtacagggttaaagcgctacctgctgacggttatggcagctgccgctaaagcctttaaacacccaagcatccgaaaccctgtcaacttggtggtgacgcgcctggtgatcctggggtccggccaggaagggccccaagtggggccaagtgccgcccagaccctacgcagcttctgcacctggcagcggggcctcaacacccctaacgactcagatcctgaccactttgacacagccattctgttcacccggcaggacctgtgtggggtctccacttgtgacaccctgggtatggctgatgtgggcacagtgtgtgatccagctaggagctgtgctattgtggaagatgatgggctccagtcagccttcactgctgctcatgaactgggccatgtcttcaacatgctccatgataactccaagccatgcactaacttgaatgggcaggggggttcctctcgccatgtcatggctcctgtcatggcccatgtggaccctgaagagccctggtcgccctgcagtgcccgattcatcactgacttcctggacaatggttatgggcactgcctcttagacaaaccggaggctcccctccatctaccagcgacttttcctggcaaggactatgacgctgaccgccaatgccaactgaccttcggtcctgactcaagccattgtccacagctgccaccgccctgtgctgccctctggtgctctggccacctcaatggccatgccatgtgccagacgaagcactcaccttgggctgatggcactccctgcgggtcttcacaggcctgcatgggtggccgctgtctgcacgtggaccagctcaaggacttcaatgttcctcaggctggaggctggggcccctggggaccatggggtgactgctccaggacttgtgggggtggtgtccagttctcctcccgggattgcacgaggcccgtcccccggaacggtggcaagtattgtgagggccgccggactcgcttccgctcctgcaacacggagaactgcccacacggctcagcattgaccttccgtgaagagcagtgtgctgcctacaaccaccgaaccgacctcttcaagagctttccagggcccatggactgggttccgcgctacacaggtgtggcccctcgagaccaatgcaaactcacctgccaggcccgggcactgggctactactacgtattggagccccgggtggcagatgggactccctgctccccagacacctcctctgtctgtgtccagggccgctgtatccatgctggctgtgaccggatcattggctccaaaaagaaatttgacaagtgcatggtgtgcggcggggatggctctcgctgcagcaagcagtcgggctccttcaaaaaattcaggtatggatacagcgatgtggtcacgatccctgcgggggccacccatatccttgtacggcagcagggggggtctggtctcaagagcatctacctggccctgaagctttctgacggttcttacgccctcaatggtgaatacacgctgatgccctccccaacagatgtggttcttcctggggcagtcagcttgcgctacagcggagccacagcagcctcagagacactgtctggacatgggccgctggcccagcccttgacgctgcaagtcctggtggctggcaacccacagaatgcacgtctgcggtacagtttctttgtcccgcggccagtcccttcaacaccacgccctcctccccaagactggctgcaacgcagggcagagatactgaagatccttcggaagcgtccctgggcaggccggaaataacctcactgtcccggctgccctttttgggcgccggggcctcggactcatctgggagaatgagcaggcttctgcaactgcctcctgctaaaacacagtagggaggtgtagagggtgagatctgcctgcctcactgccccaaaccgcaggctggccctgccctggcttcctgccctgggaggcagtgatgtcttggtgaatggaaaggggctaggtgacagtaccctatctactaaactgccccctctaccctgcaggtcacaggaggaatggggggaagacagggtgggtcctgggccctagttgtatttatttggtatttattcatttttatttagcaccaggaaaggggactagggtcttggggaaactcacctattatagccctaacctagctatgaaatccagggtgttggtgacaaatatgagtggtgtgtgtgtgtgtgtgtgtgtgtgtgtgtgtgtttatgtatgaggtacaacctgccctgctttcctctccctaatttttttttttttctgggaaaaggaaagtcaaaggtaggactgccttcagggagtaagggatgattgtgtttttaaattgaagtttgctatttatatgctctttttggagtcagacaaatgtgggttatattctggccccgcatctttgagcattagttttctcatgtgccaataataatcccttagaaattggttgtaaggattaaatgatgttaataaagaactagcatagagcctctc

SEQ ID NO: 22 - protein, 845 aa, Mus musculus
MASIHPSCSPGTMSQMGLHPRRGLTGHWLQRFQPCLPLHTVQWRRLLLLAFLLSLAWPASPLPREEEIVFPEKLNGSSILPGSGVPARLLYRLPAFGEMLLLELEQDPGVQVEGLTVQYLGQAPEMLGGAEPGTYLTGTINGDPESVASLHWDGGALLGVLQYRGAELHLQPLEGGALNSAGGPGAHILRRKSPASSQGPMCTVKAPSGSPSPISRRTKRFASLSRFVETLVVADDKMAAFHGTGLKRYLLTVMAAAAKAFKHPSIRNPVNLVVTRLVILGSGQEGPQVGPSAAQTLRSFCTWQRGLNTPNDSDPDHFDTAILFTRQDLCGVSTCDTLGMADVGTVCDPARSCAIVEDDGLQSAFTAAHELGHVFNMLHDNSKPCTNLNGQGGSSRHVMAPVMAHVDPEEPWSPCSARFITDFLDNGYGHCLLDKPEAPLHLPATFPGKDYDADRQCQLTFGPDSSHCPQLPPPCAALWCSGHLNGHAMCQTKHSPWADGTPCGSSQACMGGRCLHVDQLKDFNVPQAGGWGPWGPWGDCSRTCGGGVQFSSRDCTRPVPRNGGKYCEGRRTRFRSCNTENCPHGSALTFREEQCAAYNHRTDLFKSFPGPMDWVPRYTGVAPRDQCKLTCQARALGYYYVLEPRVADGTPCSPDTSSVCVQGRCIHAGCDRIIGSKKKFDKCMVCGGDGSRCSKQSGSFKKFRYGYSDVVTIPAGATHILVRQQGGSGLKSIYLALKLSDGSYALNGEYTLMPSPTDVVLPGAVSLRYSGATAASETLSGHGPLAQPLTLQVLVAGNPQNARLRYSFFVPRPVPSTPRPPPQDWLQRRAEILKILRKRPWAGRK

SEQ ID NO: 23 - DNA, 3002 bp, Mus musculus
  CDS (18)..(2810)
ccggcgggcagcgcactatgcggctcgagtgggcgtccttgttgctgctactgctgctgctgagcgcgtcctgcctgtccctggccgctgacagccccgccgcggcacctgcccaggataaaaccaggcagcctcaggctgcagcagcggccgccgagccggaccagccgcagggggaggaaacacgggagcgaggccatttacaacccttggccgggcagcgcaggagcggcgggctggtccataatatagaccaactctactctggcggtggcaaagtgggctaccttgtctacgcgggcggccggaggttcctgctggacctggagagagatgacacagtgggtgctgctggtagcatcgttactgcaggaggagggctgagcgcatcctctggccaccggggtcactgtttctacagaggcaccgtggacggcagccctcgatccctagctgtctttgacctctgcgggggtctcgatggcttctttgcagtcaagcatgcgcgctacactctaaagccactcctgcgtgggtcctgggcagagtatgaacgaatttatggggatggatcttcccgcatcctgcatgtctacaaccgcgagggctttagcttcgaggccctgccgccacgcgccagttgcgagactcctgcatccccatctgggccccaagagagcccctcggtgcacagtagatctaggagacgctcagcgctggccccgcagctgctggaccactcagctttctcgccatctgggaacgcgggacctcagacttggtggaggcgtaggcgccgttccatctccagggcccgccaggtggagctcctcttggtggctgactcgtccatggccaggatgtatgggcggggcctgcagcattacctgctgaccctggcctccatcgccaacaggctgtacagtcatgcaagcattgagaaccacatccgcctggcggtggtgaaggtggtggtgctgacggacaaggacacgagtctggaggtgagcaagaatgcggccacgaccctcaagaacttttgcaaatggcagcaccaacataaccagctaggggatgatcacgaagagcactacgatgcagccatcctgttcacccgagaggatttatgtgggcatcattcatgtgacaccctgggaatggcagacgttgggaccatatgttctccggagcgcagctgtgcagtgattgaagatgatggcctccatgcagccttcactgtggctcatgaaattgggcatctacttggcctttctcatgacgattccaaattctgtgaagagaacttcggtactacagaagacaagcgtttaatgtcttcaatccttaccagcatcgatgcatccaagccctggtccaaatgcacgtcagccaccatcacagaattcctggatgatggtcatggtaattgtttgctagacctaccacggaagcagattttgggtcccgaggaactcccaggacagacctacgatgccacccagcagtgcaacttgacatttgggcctgagtactcggtgtgccctggcatggatgtctgtgcgcggctgtggtgtgctgtggtgcgccaaggccaaatggtgtgtctgaccaagaagctgccggctgtggagggcactccctgtgggaagggaagagtctgccttcaaggcaaatgtgtggacaaaactaagaaaaaatattactcgacatcaagccatggaaattgggggtcctggggcccctggggtcagtgttctcgctcatgcgggggaggagtgcagtttgcctaccgccattgtaataaccctgcacctcgaaacagtggccgctactgcacagggaagagggccatataccgttcctgcagtgttacaccctgcccacccaatggtaaatcttttcgccatgagcagtgtgaagccaaaaatggctatcagtctgatgcaaaaggagtcaaaacatttgtagaatgggttcccaaatatgcaggtgtcctgccggcagatgtgtgcaagcttacctgcagagctaagggcacaggctactatgtggtcttttctccaaaggttacggatgggactgaatgcaggccgtacagcaactctgtgtgtgtccgaggacggtgtgtgagaactggatgtgacggcattattggctcaaagctacaatatgacaagtgtggagtgtgcggaggggataactccagttgtacaaagattatcggaaccttcaataaaaaaagcaagggttatactgacgttgtgaggatccctgaaggagcaacccacataaaagtccgacagttcaaagccaaagaccagactagattccctgcctacttagccctgaagaagaaaactggcgagtaccttatcaatggcaagtacatgatttccacttcagagaccatcatcgacatcaatggtaccgtcatgaactacagtggatggagccacagagatgattttttacatgggatgggctattcagccacaaaagaaatcctgatcgtgcagatccttgccacagacccaactaaagcgctaggcgtccgttacagcttttttgttcccaagaagaccactcaaaaagtaaactctgtcatcagccatggcagcaacaaggtgggaccacactctacacagctgcagtgggtgacaggtccatggctggcctgctccaggacctgtgacacaggctggcacactaggaccgtgcagtgccaggatggaaacaggaaattagctaaaggatgccttctctctcagaggccttctgcatttaagcaatgtctgctgaagaaatgttagcctgtggtttactctaatgcacaaaaaaacaacaggaggatcatcgcagatacagctgtggtgaagacaaggcctacccaaagcacagaaagtcatgccttcatgtcattgtcaccacgagtcgaattatgggcagaatctgctctctgcgaccaaaaggtttactctacttggtgaatgatggtaccgtga

SEQ ID NO: 24 - protein, 930 aa, Mus musculus
MRLEWASLLLLLLLLSASCLSLAADSPAAAPAQDKTRQPQAAAAAAEPDQPQGEETRERGHLQPLAGQRRSGGLVHNIDQLYSGGGKVGYLVYAGGRRFLLDLERDDTVGAAGSIVTAGGGLSASSGHRGHCFYRGTVDGSPRSLAVFDLCGGLDGFFAVKHARYTLKPLLRGSWAEYERIYGDGSSRILHVYNREGFSFEALPPRASCETPASPSGPQESPSVHSRSRRRSALAPQLLDHSAFSPSGNAGPQTWWRRRRRSISRARQVELLLVADSSMARMYGRGLQHYLLTLASIANRLYSHASIENHIRLAVVKVVVLTDKDTSLEVSKNAATTLKNFCKWQHQHNQLGDDHEEHYDAAILFTREDLCGHHSCDTLGMADVGTICSPERSCAVIEDDGLHAAFTVAHEIGHLLGLSHDDSKFCEENFGTTEDKRLMSSILTSIDASKPWSKCTSATITEFLDDGHGNCLLDLPRKQILGPEELPGQTYDATQQCNLTFGPEYSVCPGMDVCARLWCAVVRQGQMVCLTKKLPAVEGTPCGKGRVCLQGKCVDKTKKKYYSTSSHGNWGSWGPWGQCSRSCGGGVQFAYRHCNNPAPRNSGRYCTGKRAIYRSCSVTPCPPNGKSFRHEQCEAKNGYQSDAKGVKTFVEWVPKYAGVLPADVCKLTCRAKGTGYYVVFSPKVTDGTECRPYSNSVCVRGRCVRTGCDGIIGSKLQYDKCGVCGGDNSSCTKIIGTFNKKSKGYTDVVRIPEGATHIKVRQFKAKDQTRFPAYLALKKKTGEYLINGKYMISTSETIIDINGTVMNYSGWSHRDDFLHGMGYSATKEILIVQILATDPTKALGVRYSFFVPKKTTQKVNSVISHGSNKVGPHSTQLQWVTGPWLACSRTCDTGWHTRTVQCQDGNRKLAKGCLLSQRPSAFKQCLLKKC

SEQ ID NO: 25 - DNA, 3077 bp, Mus musculus
  CDS (59)..(2200)
ccacgcgtccggccagccccagggaccgcagcaatctcccaaactccccttccccaggatgacagaggagttaatcacccctgtgtactgcaccggggtgtcagcccaagtacagaagaagcgggacaaggagctgggcttgggccgccatgaaaacgccatcaagtacctgggccaggattatgaaacgcttcgggcaagatgcctgcagagtggggtcctcttccaagacgaggccttccctccggtttctcatagcctgggcttcaaggaactgggtcctcattcctctaaaacctatggcatcaaatggaagcggcctacggaactgatgtcaaacccccagttcatcgtggatggagccacccggacagacatctgccagggagcactgggggactgttggctcctggctgccattgcctccctcaccctcaacgagactattctgcaccgagtggttccctacggccagagctttcaggatggctacgctggcatctttcatttccagctgtggcagttcggggagtgggtcgacgtggtcatagatgacttgctgcccaccaaggacgggaagctggtgttcgtgcactctgcccagggcaacgagttctggagcgcactgctggagaaagcctatgctaaagtgaatggcagctatgaggctctttcgggaggctgcacctcagaggccttcgaggactttaccggtggggtcactgagtggtacgacctgcagaaggcccccagcgacctctaccagatcattctaaaggccctggaacgaggctccttgctgggctgctccattaatatctccgatatccgtgatttagaggctattacttttaagaacctggtgaggggccatgcgtactctgtgacgggcgccaagcaggtaacttaccagggccagcgggtgaacctaatccggatgcggaacccctggggtgaagtggagtggaaaggaccctggagtgacagctcctatgagtggaacaaagtggacccctatgaacgagagcagctgagggtcaagatggaggatggggagttctggatgtcgttccgagacttcatccgtgaattcaccaaactggaaatctgcaaccttacaccggacgcccttaagagcaggaccctccggaattggaataccacattttacgagggcacctggcgtcggggaagcaccgctggaggctgcaggaactacccagctaccttctgggtaaacccccagttcaagatccggttggaggaggtggatgacgcagacgactatgacaaccgggagtcgggctgcagcttcttgctggccctcatgcagaaacaccgccgcagggagcgtcgctttggccgggacatggagaccatcggttttgcagtgtaccaggtccctcgggagctggcgggtcagcctgtgcacttgaagcgtgatttcttcctggccaacgcttctcgggcgcagtcagagcacttcatcaaccttcgggaagtcagcaaccgtatccgcctgccgcccggggagtacatagtggtgccctccaccttcgagcccaacaaagaaggcgacttcctgctgcgcttcttctcagagaagaaggctgggacccaggaactagatgaccagatccaggccaacctccctgatgagaaagttctctctgaagaggagattgatgacaacttcaagaccttgttcagcaagttggcaggggatgacatggagatcagcgtcaaggagctacagaccatcttgaacaggatcattagcaaacacaaagacctgcgcactaatggcttcagcctggagtcgtgccgcagcatggtgaacctcatggatcgagatggcaacgggaagctgggtctggtggagttcaacatcctgtggaaccgcatccgaaattacctgaccatcttccggaagtttgacctggacaagtctggcagcatgagtgcctatgagatgaggatggccatcgaggctgcaggcttcaagcttaacaagaagctgcatgaactcataatcacccgctactcggagcccgacctggccgtggactttgacaactttgtgtgctgtcttgtgcggctggagaccatgttccggtttttcaaacttctggacacagacctggatggtgttgtgacctttgatctatttaagtggctccagctgactatgtttgcctgaagtggggaatcgctccgctccagttgcctttgttcttccattcttcctctgccaagccacgcctccccgccaagccacgcctccccgccgtgccactccagactacaccagcttctggtgccttcctcagaacaggaaggaaggcacccttgtcctcctgttccccttgtcctcctgtcctttcacctccccactacctctgttcgactgctctgggcagtcacggggccctccctgcccctgtagtcagactgggcaccctgacccctgctcagggctaagaccaggaagacagctccctgctcatccctgcctcggggctggggcaggggcaggggcaggggcaggggcaggcacccctgcccctccccctcgctgctactgtccggatgtgagtcttgcctaatctgtggaggctctagcccctgtcatcccctagagctgcagacttcataaccactagctagctccctgtctgcacccgggtgttgaaggcctcacctcactgtgggagccccacagccagactcaccttcctgtgccttctgtgcagcagccaagatccctctgcaaaccaaactacacctctcctagcctgtgtgcccaccaggaacttcctggccccgagggtgctcaggccctccccatccccgacccctctccctctctttttttgtatatatactggtcattttcaagggaatgcctgagggatgagtgtactggttatggaggaactgggggtggggtagggaggtcccgtgtttcagtgcagtgagaaaataaaatgaaaggctgtaaaaaaaaaaaaaaaaaaaaaaaaaaaaaaaaaaaaaaaaaaaaaaaaaaaaaaaaaaaaaaaaaaaaaaaaaaaaaa

SEQ ID NO: 26 - protein, 713 aa, Mus musculus
MTEELITPVYCTGVSAQVQKKRDKELGLGRHENAIKYLGQDYETLRARCLQSGVLFQDEAFPPVSHSLGFKELGPHSSKTYGIKWKRPTELMSNPQFIVDGATRTDICQGALGDCWLLAAIASLTLNETILHRVVPYGQSFQDGYAGIFHFQLWQFGEWVDVVIDDLLPTKDGKLVFVHSAQGNEFWSALLEKAYAKVNGSYEALSGGCTSEAFEDFTGGVTEWYDLQKAPSDLYQIILKALERGSLLGCSINISDIRDLEAITFKNLVRGHAYSVTGAKQVTYQGQRVNLIRMRNPWGEVEWKGPWSDSSYEWNKVDPYEREQLRVKMEDGEFWMSFRDFIREFTKLEICNLTPDALKSRTLRNWNTTFYEGTWRRGSTAGGCRNYPATFWVNPQFKIRLEEVDDADDYDNRESGCSFLLALMQKHRRRERRFGRDMETIGFAVYQVPRELAGQPVHLKRDFFLANASRAQSEHFINLREVSNRIRLPPGEYIVVPSTFEPNKEGDFLLRFFSEKKAGTQELDDQIQANLPDEKVLSEEEIDDNFKTLFSKLAGDDMEISVKELQTILNRIISKHKDLRTNGFSLESCRSMVNLMDRDGNGKLGLVEFNILWNRIRNYLTIFRKFDLDKSGSMSAYEMRMAIEAAGFKLNKKLHELIITRYSEPDLAVDFDNFVCCLVRLETMFRFFKLLDTDLDGVVTFDLFKWLQLTMFA

SEQ ID NO: 27 - DNA, 2105 bp, Mus musculus
  CDS (418)..(1671)
gagccttcccggcgcgtgagccggatccggtggcaccgcggggaagagacaggaccgggcggtggcggcagagacagggggacgcacccggtgcagaagatccaataggagcacgccgccgcaacctctcccgcgcgctccggtcgccgactctacgccgatcgcccactccccgcaccttggactacaccgggaaaaagacgcagcctggcccgaactggggcggagatccttcttgcttcccaagagctcgggagggaagattctcccgccgccgagaaccccgccggactggaggaacccgtggcctggagagcgctggctgtgccagaccccagcctgatggatgtctggtgtggataatgagggaagaacgtgccttttacacccaagaggtgaccccggagcgtgccccggatgaccccacaactcggaacgatgcggctagcctgcatgttctcgtccatcctgctgtttggagctgcgggcctgctcctcttcatcagcctccaggaccctatagagctcagcccccagcaagttccaggtataaagttcagcatcaggccccagcaaccccagcatgatagccacttgaggatatccacagaaaagggcacacgagattcacccagcgggtcgccaagaggcctccagctgcaagcgcctgaccaacctcgacctcacccgaaggcagcgggatctcctttgcgcctccggcagcgcaggcggagactgctcatcaaaaagatgccagccgcagggactaaccaaggcaacaactcgtccgaaacctttatccagccgagaccccgcaccatggacagtcgttgggtcagcctgcaccagacccaacaggagcgcaagcgtgtgatgcgagaagcctgcgctaaatacagggccagcagcagccgcagagctgtcactccccgccacgtctcccgcatcttcgtggaggaccgccaccgtgtactgtactgtgaagtacccaaggcaggctgctccaactggaagagggtgctcatggtgctggcagggttagcctcatccacggcagatatccaacacaacaccgtccactatggcagcgcccttaagcgcctggatacttttgaccggcagggcatagtgcaccgcctcagtacctacaccaagatgctctttgtccgggaaccctttgagcggctggtctctgctttccgagacaagtttgagcatcctaacagctactatcatcctgtctttggcaaggctatcctggcccggtaccgcgccaacgcctcgcgggaggcactgcggactggctccggtgtgcagttccccgagttcgtccagtacctgttggatgtccaccggcccgtgggcatggacatccactgggaccatgttagccggctgtgcagcccctgcctcatcgactatgactttgtgggcaagttcgagagcatggaagacgatgccaacttcttcctgcgtctcatccatgcgcccgggaacctgactttcccgaggttcaaggacaggcactccgaggaggcgcggaccacatcgagaatcacccatcagtacttcgctcagctctcctcgctgcagcgacagcgaacctacgacttctactacatggattacctgatgttcaactactccaaacctttctcggacctgtactgagggcggggcctgctggtcaggggcggggtctgccggtcatgcccactcacctgcgcataggcggcctccggggactgagctctgaggatgtgaggccttgtggctgtggccctagggtgggccacagaggcccagacaatggaccttgacccttgtcccacacccatttcctcattgggttggctgagtttgagacggagcacgactcggatggatgctttaagaactcagctgagctatgctgagctatgctgtccagggaagcctgagacccagaagagggccccagcgtcgagggatgtcctacatccccttatcctttgccttgtaccaaaccacgtggtttgctgcttttctatgacccagggtcatctgaataaagcacatggttttcagagcaaaaaaaaaaaaaaaaaaaaaaaaaaaaaaaaaaa

SEQ ID NO: 28 - protein, 417 aa, Mus musculus
MTPQLGTMRLACMFSSILLFGAAGLLLFISLQDPIELSPQQVPGIKFSIRPQQPQHDSHLRISTEKGTRDSPSGSPRGLQLQAPDQPRPHPKAAGSPLRLRQRRRRLLIKKMPAAGTNQGNNSSETFIQPRPRTMDSRWVSLHQTQQERKRVMREACAKYRASSSRRAVTPRHVSRIFVEDRHRVLYCEVPKAGCSNWKRVLMVLAGLASSTADIQHNTVHYGSALKRLDTFDRQGIVHRLSTYTKMLFVREPFERLVSAFRDKFEHPNSYYHPVFGKAILARYRANASREALRTGSGVQFPEFVQYLLDVHRPVGMDIHWDHVSRLCSPCLIDYDFVGKFESMEDDANFFLRLIHAPGNLTFPRFKDRHSEEARTTSRITHQYFAQLSSLQRQRTYDFYYMDYLMFNYSKPFSDLY

SEQ ID NO: 29 - DNA, 1369 bp, Mus musculus
  CDS (40)..(1281)
attcagacatgaagagagacacagtggtctgaagtggtcatgaaagccaaacaagtcttcttttctgtcctgctgtttgggacagcagggcttctgctcttcatgtacttgcaagcatggattgaagaacatcatacagggaaaatagagaagaaaagggatcagaaaggagtatcggtgactacgggaaaaatccagaaacagatcacgaatcagaactctgaggttcacatgcctgaagatctgaagaagaaagggggagatctgctcaacctagggagtccaacaagggttttaaggaagatcagccattcacaaagggagaacggagcttacagatcaactgaagcacatcaaggagctaaaattgaagtttttcagaaacccatccagatggactggccactggtcactcagcccttaaacaaaagtttggtccaaggcaacaaatggaagaaagcagatgcaacccaagagaagcgtcggtcattccttcatgagttttgcaagaaatatggtagagtaaatgatcccaagttcaacctttttcatatagtatctaggatatatgtagaagacaaacacaaaatcctgtactgtgaagtaccaaaagctggctgctctaattggaaaagaattctgatggtcctaaatggattggcttcctctgcatacaatatctcccatgatactgtgcactatgggaagcatctgaaaacactggatagttttgacttaaaaggagtacacatgcgtttgaatacatataccaaagctgtgtttgttagagatcccatggaaagattagtctccgcatttagggataaatttgagcatcccaatagttactaccatccggtgtttggaaaggcaattatcaagaaatatcgaccaaatgcctctgcagaagcattaaataatggatctggagtcaaattcaaagaattcgcctactatttgctggatgctcaccgtccagtaggaatggatattcactgggaaagagtcagcaaactgtgttatccgtgtttgatcaactatgactttgtagggaagtttgagaccttaggagaggatgccaattactttctacagttgattggtgctccaaaagagttgacatttccaaactttaaggataggcactcctctgatgaaagaaccaatgcccacgtggtaaggcagtatttaaaggacctgagcacagccgaaagacagctcatctatgacttctatcacttggactatttgatgtttaattacacaactccacatttgtaatttgcattcatttttctaaagccctacatagatttaatgatgatggcctcaaataagctactgtaattgtcctacaattctctgtatg

SEQ ID NO: 30 - protein, 413 aa, Mus musculus
MKAKQVFFSVLLFGTAGLLLFMYLQAWIEEHHTGKIEKKRDQKGVSVTTGKIQKQITNQNSEVHMPEDLKKKGGDLLNLGSPTRVLRKISHSQRENGAYRSTEAHQGAKIEVFQKPIQMDWPLVTQPLNKSLVQGNKWKKADATQEKRRSFLHEFCKKYGRVNDPKFNLFHIVSRIYVEDKHKILYCEVPKAGCSNWKRILMVLNGLASSAYNISHDTVHYGKHLKTLDSFDLKGVHMRLNTYTKAVFVRDPMERLVSAFRDKFEHPNSYYHPVFGKAIIKKYRPNASAEALNNGSGVKFKEFAYYLLDAHRPVGMDIHWERVSKLCYPCLINYDFVGKFETLGEDANYFLQLIGAPKELTFPNFKDRHSSDERTNAHVVRQYLKDLSTAERQLIYDFYHLDYLMFNYTTPHL

SEQ ID NO: 31 - DNA, 4264 bp, Mus musculus
  CDS (561)..(2246)
ggagtcaggttgacgcgccctgggtgcggggtgctaggcgcggggctgggcgcgcgctcccgcggagtcggagctgcagaggcttccgagatcgtcttgggcttttcaggtcacctagcattggtatgaatctgctcctcaagaagagaaaatattctgaaggcatgtccgtttacctgtcaacgaggaacccataatgtcttctcatctccagtgtggctccatgctctctcctgatgctactccaactttctttggtgccatgggaaaaacctgaagcacgagcagagtcacctccaaatctgtgctcacttcattttgttgcatggctggtgcggaacagactattggaatttgctcttgtgtgttgcccttcgtacctgaaccaaagactgtgcctcattctctagtgcattgctgcctgctgtcttcctcgttcatttctgttgcttgtgccttcctactgtcttctgtcatcacaggacacccttaaagtttctcactggactcctcgagggctggcccatggctctctaagccagctgtccaaggtctctaccatgaggcattgcattaattgctgcgtccagctgttccccgaagacacacacaaacagcaagttgcctgccaaggaggcccccatcacagtcatcaggcgtgccccacttgcaaaggagaaaacaaaattctgtttcgtgtggacagtaagcagatgaacttgcttgctgttctcgaagtgaggactgagggcaatgaaaactggggtgggtttttgcgcttcaggaaggggaagcgatgtagcctggtctttggattgataataatgaccttggtgatggcttcttacatcctttccggggctcatcaggaacttctgatttcctcccctttccattatgggggctttcccagcaaccccagcgtgatggatggtgagaaccccagtgatgtgaaagagcatcactaccagccttctgtaaataacatctcctatgtaaaggactatccgagcattaaactgattatcgacagcattgctgccaggattgagttcacaaccaggcagctcccggacttacaagatctcaagagacaagagttgcatatgttttctgtaatccccagcaaattcctcccgactagcaagagcccttgttggtatgaggagttctcgggcaggaacaccactgacccctacctgaccaattcctacgtgctctactccaagcggttccgctctacctttgacgctctgcgcaaggttttctggggccacctgtcccacgtgcagggcaagcacttccgcctgcgctgcctgccccacttctacatcattgggcagcccaagtgtggaaccactgacctgtacgaccgcctccggctgcatccagaagtgaaattctcagccatcaaggagccgcactggtggacccggaagcgctttggaattgtccgcctgcgggacggactacgagaccgctaccctgtggaagattacctggacctctttgacttggctgcacaccagatccatcaaggactgcaggctgcctctgcagagcagcccagcaagatgaataagatcattattggagaggccagcgcctctacaatgtgggataacaatgcctggaccttcttctatgacaacagcacagacggcgagccaccattcctgacccaagacttcatccatgcctttcaaccagaagccaagctcattgttatgctcagggatcctgtggagaggttgtactcagactatctctacttcgcaagttccaataaatctgcagatgacttccacgagaaagtgacggaggctctgcagctgtttgaaaattgcatgctggattattcactgcgcgcctgcgtctacaacaacaccctgaacaatgccatgcccgtgaggctccaggttggcctgtatgctgtgtacctcctggattggctgactgtcttcagtaaggagcagttcctcattctccgtctggaagaccacgcatccaatgtcaagtataccatgcacaaggtcttccagtttctaaacctggggcctctgagtgagaagcaggaagctctgatgaccaagagccctgcatccaacacacggcgtcctgaggatcgtagtctgggacccatgtggcccatcacccagaagattctgcgtgaattctatgggcctttcaacacaaggctggcacaggtcttggatgacgaagcatttgcctggaagacaacgtgagagctgagtcccttctgcagaagctgggcccactgactctgtcattgccatgactttaaaagtctccaggcggagaactgtcacccacagcgtggagaaagccgctggagccccacccaatcctcttctcatgtccaaggcacctggtgtgggaactattttaaagctttccttgccatgttcagcagctctgctcttgattgcaaggctcgtccacctgaatccttggaaacgacactgggggtaggcttctgtctgttcccagagtgccttggatctcaggagggcagtggatctctggcacccagtgggtaggaagaggaagctacgggaggccatagaccagccttgccttccttcctccaggagagtttgtctgggtgtagggttggacagctctgggatccgattaaggccatgttctggggctgcccctcacgtgttatatcccagcaggccttgcataaatcgacaaccccatgtggacagagaacagagctgcttggagaatggaggctctttcgtttttgttcgtattcccaagtcagcccttcggtgacatcagaaatgagccctgttccctttgctgtgctcagaagaaactcagaagtcagcttttggcagtaggctcgtctctctgacctattctgcccatacactttgtttatgcccagcgtagagaaggagcgttttaaaacaaactgcttctcgcatttgactggccctgcctctcttttctgctcccttggatgttttccttaaggacttcagtttatatttggaaagggaagttttgctgttaatgccagaacagaacacctcaaggcatcgaacagtggaaaaggaaggaagtgaagccctccccatttgtgggggtcactggagagtgtggggatgtttttggttctggctggttacaggctcccccctgtcttcctgttggaggaatctttatccacattctctgtcatgtgcagccaaagttctgtaactcactaagggtgagtctcaagacacccttagaatgtcctcagaaacacatctcacaaactaagctcgggccacatgtggtgtcactaggagatagctttgatttttatgaacctttccaggcctgctagcagcttggttcgcaaaggatatgctgaaagtcttgtcattttaaaaaaaaaaaaagaccccagggaataccagagagaacaacttaaacccacttggcacacaggacaaggtggggatgtcacaaagcttttcattggagcgttgccaagggatgctgcctgtgaagctgtgttctcaggtcaggatcctgttcaccagcgtcagaaagtgtacacaccacatgcccgactgtcacttggacgtgtgagaaaagctgggctttggcagcaggaggctgtgcagttgctcgactgtgtgagttctgctgcggtccacccttgttggagatgggactctcagctcattggaaggttccgggagagctctgggcatcagcatggactgggctcgttctgctactgaaggggctgtgtgtcatgtggcctgtgttccaggacaattctcccttctctcacggtggccacttggctcggcagccagtgggaaaatctagcctgagtacaaggatgctttgggaagaaatgacgagagagagagagagagagagagagagagagagagagagagagagagcgagagagagcgcaagctcaagcgcaagcgtgtgagaaagagcacgagagagtatttatttggaataccgtgtgtgtgtgtgtgtgtgtgtgtgtgtgtgtgtgtgtgtgtgtgtgtgcgtgcgcgcgcgagtgtgcgcgcgaacactcgtgcttatactgagcagggccctctcagctgtcttgttgatggggcttctcctccattagagacatgatgcctttatgaattaatttctccaacccttcgcctgtcatttccaggacatatcttactgccccacaccaaccacgactctaagagattttttctgacgcactccc

SEQ ID NO: 32 - protein, 561 aa, Mus musculus
MRHCINCCVQLFPEDTHKQQVACQGGPHHSHQACPTCKGENKILFRVDSKQMNLLAVLEVRTEGNENWGGFLRFRKGKRCSLVFGLIIMTLVMASYILSGAHQELLISSPFHYGGFPSNPSVMDGENPSDVKEHHYQPSVNNISYVKDYPSIKLIIDSIAARIEFTTRQLPDLQDLKRQELHMFSVIPSKFLPTSKSPCWYEEFSGRNTTDPYLTNSYVLYSKRFRSTFDALRKVFWGHLSHVQGKHFRLRCLPHFYIIGQPKCGTTDLYDRLRLHPEVKFSAIKEPHWWTRKRFGIVRLRDGLRDRYPVEDYLDLFDLAAHQIHQGLQAASAEQPSKMNKIIIGEASASTMWDNNAWTFFYDNSTDGEPPFLTQDFIHAFQPEAKLIVMLRDPVERLYSDYLYFASSNKSADDFHEKVTEALQLFENCMLDYSLRACVYNNTLNNAMPVRLQVGLYAVYLLDWLTVFSKEQFLILRLEDHASNVKYTMHKVFQFLNLGPLSEKQEALMTKSPASNTRRPEDRSLGPMWPITQKILREFYGPFNTRLAQVLDDEAFAWKTT

SEQ ID NO: 33 - DNA, 1718 bp, Mus musculus
  CDS (374)..(1564)
acaaagcccggggggtccacgctggcgctggaggcgagccgggagcgagcccggggcgcggcgggacttggggcgcagggctgctacgggacgcggggctgctagcaggacgcgcgaccaggcgtgggttgcgatcgccacccccacggcggggcccgggacattttgggccctttggggccgcagcgagcggtggggaccaagacgagcagagtaccctgagcccggcaacttcggcccctccccgcccccacccggctgccctcagcgcgtccctctccatgtgcagacagccggctaggcgctccggctggcggcggatgggtgacctcttcctggccggggcgggctgttcgaggcggcggagccggcgatgaagaagaagcagcagcagcatcccggcggcggcacggatccctggccccatggggcccctgtggggggcgcccctccgtgcctgggcagctgcaagcgccggatcccgctgctgcctttcctgcgcttctccctccgggactacggtttctgcatggctaccctggtggtcttctgcctgggctccctcttctaccagctcagcgggggaccccctcgcttcctgctcgacctgcggcagtatttgggaaattccacttacttggatgaccatggaccacctcctagtaaggtccttcccttccccagccaggtggtgtacaacagagttggcaagtgtggcagtcgtaccgtggtcttgcttctgagaatcttgtcagagaagcacggcttcaatttggtcacatcagacattcacaacaagacccggcttactaaaaatgaacaaatggaactgattaagaatataagtaccgccgaacagccctatttattcacgagacatgtccacttcctcaacttctccaggtttggaggagaccagcctgtctacatcaacatcatcagagaccccgtcagtagatttctgtctaactatttcttccgtcgctttggagattggagaggggaacagaatcacatgatccgcacccccagcatgaggcaggaggagcgctacctggacatcaatgagtgcattctggaaaactaccctgagtgttccaaccccaggctgttttacatcatcccatatttctgtggacagcaccccagatgtagggagcccggcgagtgggcgctggagcgggccaagctgaacgtgaacgaaaacttcctgctcgtggggatcctggaggagctggaagatgtgctgctcttactggaaagatttttacctcattacttcaagggtgtgctgagcatctacaaggacccagagcacaggaaagcttggcaacatgactgtgaccgtgaggaagactgtcccctcccccgaggcggtgcagattctctaccagcgaatgagatacgagtacgagttctaccactacgtcagagaacaattccacctgctcaagcgcaagcttggactgaagtctcgcgtgagcggacctcccgtgagaccacagttcttcatccccacgcccctggagacggaggagccaatcgacgacgaggagcaagatgacgaaaaatggctagaagatatttataagaggtgatacctgcgccagattgtggcttctgtggcctcatccccccttttacaaagttctttggggaagagaaattctgaagggactttcattaatgctcgagtgcattaaaaagaacaaaacatt

SEQ ID NO: 34 - protein, 396 aa, Mus musculus
MKKKQQQHPGGGTDPWPHGAPVGGAPPCLGSCKRRIPLLPFLRFSLRDYGFCMATLVVFCLGSLFYQLSGGPPRFLLDLRQYLGNSTYLDDHGPPPSKVLPFPSQVVYNRVGKCGSRTVVLLLRILSEKHGFNLVTSDIHNKTRLTKNEQMELIKNISTAEQPYLFTRHVHFLNFSRFGGDQPVYINIIRDPVSRFLSNYFFRRFGDWRGEQNHMIRTPSMRQEERYLDINECILENYPECSNPRLFYIIPYFCGQHPRCREPGEWALERAKLNVNENFLLVGILEELEDVLLLLERFLPHYFKGVLSIYKDPEHRKAWQHDCDREEDCPLPRGGADSLPANEIRVRVLPLRQRTIPPAQAQAWTEVSRERTSRETTVLHPHAPGDGGANRRRGAR

SEQ ID NO: 35 - DNA, 5091 bp, Mus musculus
  CDS (318)..(1595)
ggcggcacccaaacagctaccccgtgcggagacccaaactttctactgccacttggagtctcgcggccgccgcctccgccccgcgcgtccggggcctgcccgtcagtccgtcggtccgcgtggagcagctcgggcgccgccgtgctcccgatccccgcagctgcagcgccgcagtcctggcccggacgcccgggcaagttctccagagttaaaagcgaagttcgggcgaggcgcgggccgagctgcctctgagcgcccccggcgtccccagtgcgcccagccccgccgggggcgccggtgacccttcggtgccagccatgtcgtccatcctgcccttcacccccccgatcgtgaagcgcctgctgggttggaagaagggcgagcagaacgggcaggaggagaagtggtgcgagaaggcggtcaagagcttggtgaagaagctcaagaagacggggcagttggacgagctggagaaggccatcaccacgcagaacgtgaacaccaagtgcattaccatccccaggtcactggatggtcggctgcaggtgtcccatcggaaggggctccctcacgttatctactgccgcctgtggcgatggcccgacctgcacagccaccatgaattacgggccatggagctctgtgagtttgccttcaacatgaagaaggatgaagtgtgtgtaaatccttaccactatcagagagtagagacgccagttctacctccagtgttggtgccacgccacaccgagatcccggccgagttccccccactggatgactacagccattccattcccgagaacactaacttccctgctggcattgagccccagagcaatattccagaaaccccacctcctggctacctgagtgaagatggagaaaccagtgaccaccagatgaaccacagcatggacgcaggttctccaaacctctccccgaatccgatgtccccagcacacaataacttggacctacagccagtcacctactgtgagccggccttctggtgctccatctcctactacgagctgaaccagcgagttggggagacattccacgcctcacagccatccatgacagtagatggcttcactgacccctccaactcggagcgcttctgcctgggcctactgtccaatgtcaaccggaatgcagccgtggaacttacaaggcgacacattgggagaggtgtgcggctctactacatcggaggggaggtctttgcggagtgcctcagtgacagtgctattttcgtccagtctcccaactgcaaccagcgctatggctggcacccggccactgtctgcaagatcccaccaggctgcaacctgaagatcttcaacaaccaggaatttgctgccctcctagctcagtctgtcaaccagggctttgaggctgtctaccagctgacgcgcatgtgcaccatccgtatgagcttcgtcaaaggctggggagcagagtacaggagacagacagtgaccagcaccccctgctggattgagctacacctgaatggacccttgcagtggcttgtcaaggtcctcacccagatgggttccccgagcatccgctgttccagtgtgtcttagagacactaggagtaaagggagcgggttggggagggcgggcttggggaaaatgaccttggaagagaactccatccaacttggtcttgtcaaagaacaccgattccactcaactaaggcaccagcctgtttctgagaccacagaagaaaaccccagggatggatttatgaacagctgtgtctgctacatacacgtgcccctgtctgaaggccaagtgatggcttctgttctggtggcttgaactaacaggtggtgtatcgccacctgactccttgtttaatgacagaggtctgggatgtcacagtccaaaaggaaagtgcctttctccatggctggagtatggagtttacctttggagaagttgtaatggagcatgccctgtcccaccactctcagagagggtgtacctgtcaaactggatggcctacataggtactcccccctacccctaggatgcagagagacgggaacacgccggagggtagagctggggagaacccattcttccttggaaggatccgctgaaggtcagcgtataggtgatgtacagttcctaatatcacatcagtctcagagtgttcacaggaagcagcaagggcactcgtggagtatgtgtcctgggtgaggtggcaccacaccgaatgaatgcatctctgggagctggcaccacaaccctgatgtataggctgtgttggtttatggagacaagttgcatcaatgaattcacctagcataggctctttgaaatgtcctctgtttgataaaaaacaatcctgggtacgtatgttggctggaaaaccacaatggaccctgccactgcttcttgccctgaggtttggaagctgagagttatagaagccaattcaccaggaggtaagacatcccaggctgacatgggcaaatgaaaagggctattaaaatttttttacaccttggaaaattgcaggcttggtgcagagcgctctgtcatcttcaccctgggatgtaggattacctagctatggtaaaggattgccacagcaaactgtgacactgtgtaatgagcactgttcccagcggcaattacagagaaaacgagtgtaaattatttcaactggaaaaaagtccctttcttggctgttttagaacagggtacacaggatcgccacctgcaactggtactcgcttcttggctgctgcttctgttgtgaaaagacgagcccatgtttctgcatggatttcccatggaagtcctgtcctgctacagaggggaagaaagtgtaccctccaatgtgataaatcttctgatgcccccagactcttggagcacatcctggtgcccctcctgcaggagcctgtggcatatttccagctgggcatgctgatcctccttgagacacagatgcctgtgtgagtctccgttgatacaattctgaacccctcaggttctctgaaagggcacagaccatgggcgtgaacattgtgccgtacctgagttggtctgtggactgctgcttcagacacacgagtcctcggaactgcctggctgcctgtcacgcatgctctgagtcagaacacaccaacgctctgctgtggctcctagggaagcattcatggtcctctgttatcagcaggggtttatgtcacttgctgtccggtttcctaggggcttcctgtgccccttccccagctatcctccaggtggctagggacagtctattctgctgcaactggaaagtagagggaaccggcactgctcagagcagatggcggcttctggaggcacacagtgggagtacaccccttcatgttattggccagttgctggagaatgctgtaggagaaaattctaggcaggtctactcttggcatccctgagagtcaaaggcttggagtctaggaaaggtcacaccatgatggagaacacaggtcatttgggtacgtgtaatcaaagtgccctcccaaatcagttctccttttcgtatgaacagcatctctacttttaaagaggagttgaggatcgagaagatgacagtgcagcagtgggtgtggcctgactacatgtgctgttccagccctgggtgcccactgacaccgacccccaggcagaggcctttgtcttcagcactcctgagaagttggctctttaccccttctcctctgctgcccctccttcctgctggttcaggtagccccagccacgtgggttagagtcctgtgctggcctgccatggcagctggctaccttccagaccaactgtagagatacctggcattttgaagaaagcctagactggagagcagggcctctcttgggaaggacacaaggcgggcaaggctgctggcagacttctccactgacctgagtgtgctttttttttcccctaaatgtattgcatcaagcctcagtgcttatggagtgcagtggtcttcatcttccccaacttgcttctcagagctgggatgtattccagagcctgatgtttttattcaaaccacagaaaagttttctttaagtagcctgtgcagtcatgcatgtgcctgagttgtctggagcagaggcaaacatctgacttcattcttagccccaagctgccatttctgagtccttgagaggctgagaaggctctagctttgtactgtattcttactgtgtgactagatgcgtgagcgctttacattagaaggaacctggttagagctcgctcctcctgtctttgtgtggcatttgtgttccattaccggcccctttaagtaacggccttcacagcaccttcccagtgggtagaaagccacacaccaggatgtgggtcaaccatgaagatgtggcattgcagacgggggaacatgtggatgcatggctatcgccctgaacagtccctgcagctacttgtgttaacacagaactgatgtttagcattctgccgctttcgtatttatgtaacaattccttaaagccattcaaatggctaactatttaatttctttaggacagttgtaaaggtctctctcctgaggacaatgacttggaagaactggggcacagccagtcccagacactggtggaggctgcagtgactttttttggctcaagatccacaagcattagagtagactgggccaacaagtcaacaagtggtggcgtgtgcaaacgggctgccctagtcaagcccagtcccttcaacagtatgtctgatgcaccacaggccctccctactggaagtgggaacttcaaatggaaattggagccatcttttatcccagaagcctttgctgctgccagggcaagtgggctggtgtggactcttgtttaggaggctgaggttcttgtcactccttagccagccaggcctttagtgtctttgtaaaaagcatgtatttatagtgttttagatttttctaacttttgtatcttacagcattgtaccccattgttaaagagccgtgtcccctcttcttataaacgcccttttaataaacttttcaccgtaaagctcctgagacaggagcacagtctgt

SEQ ID NO: 36 - protein, 425 aa, Mus musculus
MSSILPFTPPIVKRLLGWKKGEQNGQEEKWCEKAVKSLVKKLKKTGQLDELEKAITTQNVNTKCITIPRSLDGRLQVSHRKGLPHVIYCRLWRWPDLHSHHELRAMELCEFAFNMKKDEVCVNPYHYQRVETPVLPPVLVPRHTEIPAEFPPLDDYSHSIPENTNFPAGIEPQSNIPETPPPGYLSEDGETSDHQMNHSMDAGSPNLSPNPMSPAHNNLDLQPVTYCEPAFWCSISYYELNQRVGETFHASQPSMTVDGFTDPSNSERFCLGLLSNVNRNAAVELTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLVKVLTQMGSPSIRCSSVS

SEQ ID NO: 37 - DNA, 1983 bp, Mus musculus
  CDS (80)..(1171)
tggagctggtcggttatggttggaactttccctgcccttcgttgactgagtgaaccagccagaaatacattcccaggaaatggcctctaaatcttggctgaattttttagtcttcctctgtggatcagcaatagggttttttttatgttctcaactcttgagtattttgttgcgagaagaggctgccattcagcctaacatgcttcacaatgaccctcatgcaaggcattcagatgacaatggacacagtcacctcaaaggacagatgaacttcaatgcagattccagccaacataaagatgagaacatagacgttgctgagaacctctatcagaaagttaaaattctttgttgggttatgacaagtcctcaaaatctagagaaaaaggccaaacatgtcaaagctacgtgggcccagcgttgtaataaagtgttatttatgagttcggaagaaaatcaggacttccctactgtgggattgaaaaccaaagaaggcagagagcaactatattggaaaacaattaaagctttccagtatgtacatgaccattatttagaagatgctgactggtttatgaaagcagatgacgacacatacgtcattgtggacaacctgagatggcttctatcaaagtatgaccctgaacaacccatttactttgggcgaagatttaagccctatgtgaagcagggatacatgagcggaggagcgggctatgtcctaagcaaggaagccttgagaagatttgttaatgcatttaaaacagaaaagtgtacacatagttcctccatcgaagacttagctctgggaaggtgcatggaaattataaatgtagaagctggagattccagagataccattgggaaagaaaccttccatccatttgtaccagaacaccacttaatcaaaggttatctaccaaaaacattttggtactggaattacaactattatcctcccatagagggtcctggatgctgttctgatattgcagtttcttttcactatgttgatgggacaactatgtatgaattagaatacctcgtttatcgtcttcgtccatatggttgtttatatagatatcaacctgccttacctgagaatatactgaaagaaattaatcaagtaaacagaaaggaagatacaaaaataaaattaggcaacccctgaaagcagaacataagtggtctacattgcactgaaggactcttgcctttctacggaaccctgaaatcccagtgaggaactcacctgaagtgaacattccatatagaaatctttcaaatggatgactataaactgaagcatttaaagagctgtgaagtttgctaaaacgtgtcttgatacagtaatatataaatataaataaatatatatgaagaatttgtgtttttaaatggtgacagaagagctagaaagactctgtttctgactgttcccaacagtgtatgttgttatcactacagttggagtggttagtttataatcaccttctgcaccacaccagttagaacacaatgctgaacagatctgccttaaagaaagaaagaaagaactgttgctcagtgttgtttctcagctgatatgtgtatgctgcaaaccagccttccagttgccgaatgaaaactttcctgtgattatattatttataattggcagtgcttcctcaagaagaataagaagggcacttaccccaaagtgcagtaattaaccacatttccaattattccgagccttgactccattagatacttagttctaaagataatcaacccgataccaagtccccattgtcccctttccaaatgtttgaccctgaataccatttttcactattaatgctaatttactcaaagcattggcagcattctgtaaatcatgcattttcaaatatgttgttccccgattcacccattgttaattgtaggtaacaattaaatttaatgctgtttggacaagcaaaaaaaaaaaaaaaaaaaaaa

SEQ ID NO: 38 - protein, 363 aa, Mus musculus
MASKSWLNFLVFLCGSAIGFFLCSQLLSILLREEAAIQPNMLHNDPHARHSDDNGHSHLKGQMNFNADSSQHKDENIDVAENLYQKVKILCWVMTSPQNLEKKAKHVKATWAQRCNKVLFMSSEENQDFPTVGLKTKEGREQLYWKTIKAFQYVHDHYLEDADWFMKADDDTYVIVDNLRWLLSKYDPEQPIYFGRRFKPYVKQGYMSGGAGYVLSKEALRRFVNAFKTEKCTHSSSIEDLALGRCMEIINVEAGDSRDTIGKETFHPFVPEHHLIKGYLPKTFWYWNYNYYPPIEGPGCCSDIAVSFHYVDGTTMYELEYLVYRLRPYGCLYRYQPALPENILKEINQVNRKEDTKIKLGNP

SEQ ID NO: 39 - DNA, 4263 bp, Mus musculus
  CDS (602)..(1576)
ccgagaaggagagagcggcacaggtttctgccgcggcgctcgaacgggagcttagggacgcagcatcctcggtgtccacccgggatcgcagctgcaccggccgacatgtgacggaggaaagccctcagcattcccggcaaccgccttcaaaggggtcccggaacgctcgcccaggccagcagctggatcctcttagccctcagcgacgcgggacaggagcggcaaggtacaacgcctggtacgttccaggctcgcactcgggaaaagagcgcagcggggaccgccgcctggcttgcactcggcacaactccgggacctgcggagtgcggctggaccgtgtccagattcccgccgacttcatccagagtgacccaagaccctagccggtcctcctgcaccccctggagttctagcttgccctgcatctgcgccgaccccacaacgcccaggtccccgcaacgccccagtccggaaggggagcgcgcttgcggcttctccctgattgcagacgctgccgctgtcctaactgcacacagaggtcccctacccggagcctagtccttgcagggggtggtgtccgagacgctggggagcgcgcaccatgaagtccgcgctgtgcagccgcttcttcatcctcctgccctggatcctgattgtcatcatcatgttggacgtggacccccgcaggcccgcgccccaactcacttcccgtccctacttctctccccatgcagtgggctgcgggggctcccgagtcccgctccgacggagtagccccgggcgtgacgctgccgagaagcggaacgagtctcggcctcagctgcagccggagccgcgcttgcccaccatctatgccatcacacccacctacagtcgcccggtgcagaaagccgagctcactcgcctggccaacaccttccgccaggtggcgcagttgcactggatcctggtggaggaccgggcgacgcgcagcgagctggtgagcagcttcctagctcgggccggcctgcccaacacgcacctgcatgtgcccacgccgcggcgctacaagcgaccgtggctgccgcgcgccaccgaacagcgcaacgcgggcctcgcctggctgcgccagaggcatcagcatcagagcgcgcaacccggcgtgctcttcttcgccgacgatgacaacacgtacagtctggagctcttccaggagatgaggaccactcgcaaggtttctgtctggcctgtgggactagttggtgggcgacgctatgaacgtccgttggtaaaaaatggcaaagttgttggctggtacaccggatggagagaagacaggccttttgccattgacatggctgggtttgctgtgagtctgcaagtcatcttgtccaatccgaaagctgtatttaagcgccgtggatcccagccagggatgcaagaatctgactttctaaagcagataacaacagttgaagaactggaaccaaaagctagtaactgcaccaaggttctcgtgtggcacactcggacagagaaggtcaatctagccaatgagccaaagtatcacctggacacagtgaacattgaggtgtagccgccaggaccagcaggtgaggacaggaagtctggagcgtgggctgctgcccattcaggtactgttcctttgccttcagcctttccttaccgccgatggacggccgtttcgacagctacctggatgctacaaaatgtgttgtcttcatttgtctgcatgtgttttctgaactggaagagttttgtagagtgaagccacatgtcactgaaaaggacaacgaagtatctcacactgctccctccctccctccctccctccctccatccctccctcccacaagccatctcaatactgctagaatgacaggttttagaagctgatgaggagtactctgtcaattaaatattcccagttaaattcttggcttcacaacatgaactatttggaaagctgatggcagtgtacactgtaggaaataaaacccacaaataaaaaggtgtgtggattcatctgtttaatatagggaaataacattttgtcaatactaggcaccataaaatgtaaacacaattactgtcataaacctggatataccttcaaggattaaaagtggctttgttttagttaccctgtgtgcatatagtgcagaaaaaggtcttcatattagcactatgtacatgagaagaggcccacattacaagaggcaatggaaatttgaattctgaaaccttcattgagttttacagtagcatccaggtttatctccgttcactaaccacgttccctgttaagcacatcataacaacagcacagtggagtgagtgatgaataaaagaatttgatacactagaaaacagtgctcagtgatacatttacattctatttatatgattaaacatttgatcatacagtacctttctacaggattactggctaatttgggggtggggtttatactgttagaggtattactaacatgataactacttcccttatatgcaaacgttagagctataattttgcaaataaaactgattatgcaagttaactgagcagcttcccaatgtggcttaggacatcatggggagtatgagcaaagctgccctccacacagatggtttctcaacctgctttcacatccaatttaatcagcacagaccaacacggtcaatcaggtcattcttaatgtgagcaaatgggggaaagaaaaaatacgttatgtacatgaataaatgggaactagatgcctttgcagcgaggaatgttggggtcggttctggatggaactctgtgacagcttcttacttccacagctgtgtgctcagcgtctgccccgatgagcttccagaccaccctgctgtcgtgctgggaggctggccaaaacctgcactagcgttagcactactgaggttcatggcagccatctgctgattcatctgcgaaacacaaaaggcttggtttacaaggaggaccctcttttcactcctgactccttagctacagccagacacttcatcagatgtgaaaattagtccagacactggatgtggactacactgaagtctttctactgaaatcagttacttttttcttacttttttctgagttaggtgttacttataaagtaatccataattagttcactttctagattaaccacaactgaaaacacatcaaagtaatctttgagtttatatctgagaccatcactcttctgtgactccaagactgagacattttagaaagtaaaacatctaaaattaccctaacatttacaaaatactcatacctctctattaacatcctaaatgaccttaatatacttcctaaacattaaaaaggtcatatagcatatgttttaggtttatgttataataacataaacagctaaaattaaatttcattatatgctatacaataaacaactacatgtacttttctttcaggctaaaatccagtatatttatcaggctcttagcacagtagttctcccactacccagtttggtgacggaacaagagatgcaggatttacctaaaatagtttcaaacctatgttcaacattatctgtgttctttgtgcaaattttttttaaaacccaagacataataagcacacatgtgtctacagacaggtagcttccatcttcccttctgctcactcttgtggccttgctgttcttcactggctctcctgtactctaacctgagtcatgtgagcctgagcatcctggtcacagaaaagctgttcctagtatctgacatcattcccgaggtttccaatgcataagcagtgtgtacgtatatacctataaaccgcagctgtcacagcacacgagcatttgtaagatacacggtgattctgtgaatgttagttagcagacattgttctgaaggtaaactaggggtcttagtggtcctggttcctgtgagatgctcggtggaccatcatgcaggaatgggaatgggaccatgagcactaagtcaaagcctctgatgtggggtaacagcagaaatgttactctaacctaagccagacagacatggaatttatcagtgaatacctgagggaagcagggtttacattctcaccatggggattcctgagccaatggatgttcctcatacatcaacaggcagtaaccacatgacagcggcagccttgatccctcataaaactgtgctttgaaaatatgaagctggatttctatctcactgataagaattaataaaaaaaaaaaaaaaaaa

SEQ ID NO: 40 - protein, 324 aa, Mus musculus
MKSALCSRFFILLPWILIVIIMLDVDPRRPAPQLTSRPYFSPHAVGCGGSRVPLRRSSPGRDAAEKRNESRPQLQPEPRLPTIYAITPTYSRPVQKAELTRLANTFRQVAQLHWILVEDRATRSELVSSFLARAGLPNTHLHVPTPRRYKRPWLPRATEQRNAGLAWLRQRHQHQSAQPGVLFFADDDNTYSLELFQEMRTTRKVSVWPVGLVGGRRYERPLVKNGKVVGWYTGWREDRPFAIDMAGFAVSLQVILSNPKAVFKRRGSQPGMQESDFLKQITTVEELEPKASNCTKVLVWHTRTEKVNLANEPKYHLDTVNIEV

SEQ ID NO: 41 - DNA, 1599 bp, Mus musculus
  CDS (175)..(1182)
gaagtggtcccaggtgtcgctcagagtcccgcgttcccgggaactagtaagggagcctggggaaggcggggcgcggcccgggcggccggggcggggccggcgggagggcggagggccgggcggacctgtccctgccggtcccggtggaccccgcctgccccggtggcaccggccatgaagctgaagctgaagaacgtgtttcttgcctacttcctggtgtcgatcgccggcctcctctacgctctggtgcagctcggccagccttgcgactgcctccctccgcttcgagctgcagctgagcagcttcggcagaaggacctgaggatatcccagttgcaagctgatctccgtcgcccaccccctgtcccagcccagccccctgaacctgaggccctgcctactatctatgtcattacccccacctacgccaggctggtacaaaaggcagagctggttcggctgtcccagaccctgagcttggtgccccgtctacactggctgctagtggaggacgctgagagccctaccccgctggtctcggggctgttggccgcctctggtctcctctttacacacctggctgtccttacccccaaggctcaacggcttagggaaggtgagccaggctgggtccggccccgaggagtggaacagcgcaataaggccctcgactggctccgaggaaaagggggtgctgttgggggggagaaggatccaccgccaccagggacccaaggagtcgtgtattttgctgacgatgacaacacctacagccgggagctctttaaggagatgcgttggactcgcggtgtctcagtgtggcctgtggggctggtgggtggcctgcgatttgaaggtcctcaggtacaggatggccgcgttgtgggtttccacacagcatgggaacccaacaggccctttcccttggacatggcgggatttgcggttgccctgcccttgctattggctaagcccaatgcccagtttgatgctactgcaccccggggccacctggaaagtagtctcctgagccaccttgtagatcccaaggacctggagccacgggctgccaattgtactcaggtactggtatggcacacccggacagagaaacctaagatgaagcaggaggagcagctacaacggcagggccagggctcagacccagccattgaggtgtgatggcaacctcaccctgacttctaccttattttaggcacataccttgtgggactgggctccaggccagcccaggatgtggttttttttttaaagacctgacccctgagaaccagaggcagcccctccaaccacagggtgtgtcccagttgcttccctcccttcccccagcctgccatgtggcactgcccacatgttggggacaagcagcttctctagtgagccagatcagccccatctggggcggagcaggacagatggactcagaaaggagggtagctgtgggaaaagggtaacttattggggacaagcatgcagtggggggctagaggagctgggctggaccctccccacctgagcatgcgatccccttcctacctctagaataaagaatctcaacccggaaaaaaaaaaaaaaaa

SEQ ID NO: 42 - protein, 335 aa, Mus musculus
MKLKLKNVFLAYFLVSIAGLLYALVQLGQPCDCLPPLRAAAEQLRQKDLRISQLQADLRRPPPVPAQPPEPEALPTIYVITPTYARLVQKAELVRLSQTLSLVPRLHWLLVEDAESPTPLVSGLLAASGLLFTHLAVLTPKAQRLREGEPGWVRPRGVEQRNKALDWLRGKGGAVGGEKDPPPPGTQGVVYFADDDNTYSRELFKEMRWTRGVSVWPVGLVGGLRFEGPQVQDGRVVGFHTAWEPNRPFPLDMAGFAVALPLLLAKPNAQFDATAPRGHLESSLLSHLVDPKDLEPRAANCTQVLVWHTRTEKPKMKQEEQLQRQGQGSDPAIEV

SEQ ID NO: 43 - DNA, 2974 bp, Mus musculus
  CDS (197)..(2203)
gataggggccccaggcttcccacagcatggagacacagggagtgtggagggtgccccccagcccacggacaacaccttcactccgaagtgtgagattgtgggcaaggacgcactgtcagcactggcccgggccagcaccaagcagtgtcaacaggagatcgctaatgtagtgtgcctgcaccaagctgggaacctaatgcccaagtctgtgccccggcactgccagctggctggcaagatgagccctggcgtccaatgggaagagatccgggcccagcagcctgttggtggccctccagtacgcatcgcctacatgctggtggttcacggccgtgctatccgccagctgaagcgtcttctgaaggccgtctaccatgagcaacacttcttttatatacatgtggacaagcgttccaactacctgtaccgggaggtagtggaactggcccagcactacgagaatgtgcgggtgacaccttggcgcatggtcaccatctggggcggggccagccttctgaggatgtatctgaggagcatgaaggaccttctggaaattcctggctggacctgggacttcttcatcaacctgagtgccactgactatccaacgaggacgaatgaggagctggtagcattcttatccaagaaccgggacaagaatttcctcaagtcacatgggcgagacaattccaggttcattaagaagcaaggcctggaccggcttttccatgagtgtgattcccacatgtggcgcctgggtgaacggcagatcccggcaggcattgtggtggatggtggctctgactggtttgtgctgacacgcagctttgtggaatatgtggtgtatacagatgaccccctcgtggcccagcttcgccagttctatacatacacattgcttccggctgagtcattcttccacacagtgctggagaacagcccggcctgtgcgagcctggtggacaacaacctgcgggtcaccaactggaaccgcaagctgggctgcaaatgccagtacaagcacatcgtggactggtgcggctgctcccccaacgacttcaagccacaggacttcctgaggcttcagcaagtctccagacccaccttctttgcccggaagttcgagtcgactgtgaaccaggaagtcctggaaattttggacttccatctatatggcagctacccacccagcacaccagccctcaaagcctactgggagaacatctacgacgtggccgatggccctggcggactcagcgacgtcctactcacagcctacacagcctttgcccgtctcagcctgcgtcatgctgccactgctgtatccccactggccactgcagtctgcaggtttgagcccagggggttgccgtccagcgtgcacctgtatttctatgacgaccatttccagggctacctggtgacgcaggcggtgcagccctcagcccaggggccagcagagacacttgagatgtggctgatgccccagaggtcgctgaagctgttggggcacagtgaccaggccagccggctccagagtctggaggttggtactgagtgggaccccaaagaacgtctcttccggaactttgggggcctgctgggaccactggatgagcctgtagccatgcagcgctgggcccggggccccaacctcacagccactgtggtctggattgaccccacctatgttgtggccacatcgtatgacatcacggtagatgcggataccgaagtcacgcagtacaagcctccactgagcctgccactgcggccgggagcctggactgtccgcttgcttcagttctgggaacccctgggtgagacccgcttcctcgtgctgccgttgacgttcaaccgcaaactacctctcaggaaagatgatgccagctggctgcatgcgggaccaccccacaacgaatacatggagcagagtttccagggcctaagtggcatcctgaatctgcctcaggcagaggccctggaggaggctgcccggaggcacacagagctcacgggttctgcacttgaggcctggacagatggggaactgagcaatttctggtctgtggcaggattgtgtgccataggaccttctgcttgtccctccctggagctctgcagactgaccagctggagctctctgtctcctgaccccaagtcagagctggggcctgtcaaagctgacgggcgactcaggtagcaggaccccagccagcacaacccggaggagccggggaattgcaccttacagacaacggagggacatctctcccacagacaagaaccagaggcccaggcagtttgcccatttgagccaacaggaacctacactgagggcagggaaggtgggtgcagtactccctactgctaacggccctgcaagagaccagcggtccactggagccatgaggaggatcttcctctgctcctcgttagtttatacttttttaataattaaaatgagtggcagagggaggacgatgtgcaatagggctcagagcagccccaggatatgggccatggccatggggagcaccacagagcatatcggtttctgctctggggaggcatgggctgcctccctctccagagcctccatccccagcctgcatctgaactgcgcactgctgaggctctgaggcccggaggacaggtgatctgtgggggctactgtggagttggactttagtgagcaggcctctcctctgggagccagggagccctagagcagagctggggccggctctcgggcttccctaggaaatgcgcgccatcagggcctgctgagatccatgaagtttccctactggaggaggaagcccactggcggcacaggaaggctgtctgtcagagctctcacaagagtcgagttgtgcctgaagctcagtgcgaagtctgaaatatgcaacagaggaaatatatctctatctctccaaaaaaaaaaaaaaaaaaaaaaaaaaaaaaaaaaaaa

SEQ ID NO: 44 - protein, 668 aa, Mus musculus
MPKSVPRHCQLAGKMSPGVQWEEIRAQQPVGGPPVRIAYMLVVHGRAIRQLKRLLKAVYHEQHFFYIHVDKRSNYLYREVVELAQHYENVRVTPWRMVTIWGGASLLRMYLRSMKDLLEIPGWTWDFFINLSATDYPTRTNEELVAFLSKNRDKNFLKSHGRDNSRFIKKQGLDRLFHECDSHMWRLGERQIPAGIVVDGGSDWFVLTRSFVEYVVYTDDPLVAQLRQFYTYTLLPAESFFHTVLENSPACASLVDNNLRVTNWNRKLGCKCQYKHIVDWCGCSPNDFKPQDFLRLQQVSRPTFFARKFESTVNQEVLEILDFHLYGSYPPSTPALKAYWENIYDVADGPGGLSDVLLTAYTAFARLSLRHAATAVSPLATAVCRFEPRGLPSSVHLYFYDDHFQGYLVTQAVQPSAQGPAETLEMWLMPQRSLKLLGHSDQASRLQSLEVGTEWDPKERLFRNFGGLLGPLDEPVAMQRWARGPNLTATVVWIDPTYVVATSYDITVDADTEVTQYKPPLSLPLRPGAWTVRLLQFWEPLGETRFLVLPLTFNRKLPLRKDDASWLHAGPPHNEYMEQSFQGLSGILNLPQAEALEEAARRHTELTGSALEAWTDGELSNFWSVAGLCAIGPSACPSLELCRLTSWSSLSPDPKSELGPVKADGRLR

SEQ ID NO: 45 - DNA, 5532 bp, Mus musculus
  CDS (369)..(1427)
ccagtgcacggcggcggcggcaccttccttctcgcatcccggtcgcccggcccgtgaggaggcagcggcggccgcaggcgccagcagaggaggcggcggagcatcgagcagaggcgaggcggaccggcaggagcgcgcggccggagccacgcatcctcacacttgcacaccaactcctgcgcctttcaccctctagaccagagtcggggcctcggagagcctcggtgaagctagcgggagccagctccggagcccagccagctctgttctgagcctccccggtgctggtacccccgcggcggggtccccgcttctgcgctcgggacgcgcctcccggacagccgggtccccgcggccaggacaaagccatgaagccggcgctgctggaagtgatgaggatgaacagaatttgccggatggtgctggccacttgcttcggatcctttatcttggtcatcttctatttccaaagtatgttgcacccagtcatgcggaggaaccccttcggtgtggacatctgctgccggaagggatcgagaagtcccctgcaggagctctacaatcccatccagctggagctatccaacactgccatcctgcaccagatgagacgggaccaggtgacagacacctgccgggccaacagtgccatgagccgcaagcgcagggtgctgacccccaacgacctgaagcacctggtggtggatgaggaccacgaactcatctactgctatgtgcccaaggtagcgtgcaccaactggaagaggctcatgatggtcctgagtggccggggcaagtacagcgatcccatggagatcccagccaacgaagcccacgtgtcggccaacctgaagacccttaaccagtacagcatcccagagatcaaccaccgcttgaaaagctacatgaagttcctgttcgtgcgggaacccttcgagaggctggtgtctgcctaccgcaacaagttcacgcagaagtacaacacctccttccacaagcgctacggcaccaagatcatccgacgccagcggaagaacgccacgcaggaggccctgcgcaagggggacgatgtcaagttcgaggagttcgtggcctacctcatcgacccccacacccagcgggaggagcccttcaacgagcactggcagacggtctactctctctgccacccgtgccacatccactacgacctcgtgggcaagtatgagacactggaggaggactccaattacgtactgcagctggccggagtgagcggctacctgaagttccccacctatgcaaagtccacccgaactaccgacgagatgaccacggagttcttccagaacatcagcgccgagcaccagacacagctgtacgaagtctacaaactggactttttaatgttcaactactcagtgccaaactacctgaagttggattagagggctgcggaggggagggggaggggtggttgggggagagggagagaatcctgctttttaatttaagatttttatttgtcaaaagaattctatggagactgggttattttgtaagttaatatgtcttcgggggagatgctgcgagcggcatggttaagaattatttaaaaattctccacggggaaggacagctgtctttgcaggggagcggggtggaatatccctgtttttagaagtggatactgcaacactgtctccaaggtgtccttgtgttctggtgaagtccacaaactgcattccataaagtctaatgaatcttatttatagttatttaaacgtggtctgtggcagcagcctgctcctgtctgtgcagaggagagtacagtctgtgcttcgctggctatggctcattgggggaggggagggggttatctcccccactgaccgtgtgtagaatcccatggtgacagctgcgggatgtgtgcctgtggccattcaagaaaccctgctagaaagaagtcaattgtccttgcactaagaaacagggatgagactggttcaaaccgattgctagcaaccccaaagtcccttgtagtaaagaaatgcaaatttttttaacccaagaagagagcagatgatagcgatttctctttaccgaaattagcaggctataagaataggctttctgtggaaatctttaagtggcactttccagtgctagcgaaggcagaataatggttgttttctagtcaataagtattgagcatctactattctccaggcttgactgcagatagcacattgagtgaaaccaggagccttggccttcagcaagctttatgtgaacacagggcatccagctcaacagtgaaccaacacccccatccctttggtcctcacacagactttagactattctcaagtggtccagagggtccaggaccctagcaaatttgcgtatttctacagttcaaatttgaaagccacacatttggatcaggccacttaactagctaagtagttcattgcccaacacaagaaatgacagaaacccttttgatcatgtatatctagtgcagtgtcttcatagtagaagcctataagccacaatattctccaagcgcaaggatggaggactgctgtgattgattagtgatgtctgccttgactaaggaaagggaggaacatttctcaagggcctcctactgtgatctccagtccacatcttgtcctgcctgccacttaaaacagtgcactgcatacatttggagactggcggagttcaagtgtgaaggtcaaaggtggagtgttggtacccacaggcacatgcccaccctccatgtcctgcattggcagagcatccaggacctccctctcctccagaaggttggaaatttgctctttcctgttccttgctgatcaatgcccaaacctattcctcatttctagttgtttctgatactttgcggttgacatttgagccacattcccatgacagccagtgtccgggtacgtagcagatgctgtacatagttctgatgataagggagctggtaggctgtaaattttgctttttgtgtttttgtggggttttgtttgttttgatgcagtatagagtgaagggggtaagaatattctgaaaaacataaaattgagtaatttattcacagaaactattaaaatggtattgttaggctcacaaggaggctgggggcagcggtaactgctacactgtctcgcagaggaaattattttatttaatgtgagagaaatgtgagccagagtggctgcctgaggctatttataaaacgcaggtaccttccccaatttctctcccctcgcctttaataaatagatttatgtcatgtgtgtgaccaagaacagtgacagatcttggctgtccctggaatagagaggcagtcatggtgctcaggccctgggcgtggtgtagctgatgcatgtcccaccgagcccaggctctccaaggcatcagtttgtggtcacatatccttagaaaacaaaattaagtcaggttcctgtctgcaggagttttcagagccttgaatacatagtctgccttgtgaaactgggagaatgtagcatttctgggctgcatgacctaggtatcctgtatggcagaccatgtcttagctggtttttcacacagtagccttcagtgagggtgatctagcctgtctgaggtctgcaaagattgacagctgcttcctagatccttccacaaatcagcagaaacagtgtccatctgggaactttatgagagtgttgctttacctagttggggtgtcttcatctggatactgccccagagagtgatcccgtccctccaagcacagcggagggaagcctagggtgttatggagtggggcaggtggcttaaggacaccttaaaggccacttggtggggataatggcctcgtggggatactcctcacttccacccacagtttggaactgttcactgtgtgcttcaggagttttaaatggagattcgtgttgatttccatggtccgcatctgtttgtcttccgtctgtggtccagtgctgtgtgcagatggtacttactccctttctctctcttacagggagaaagactgtccttctggggagagtgtatcaatacgcttccagaatgagggagtctttctcctgtcttgtgttcacacttcctagtgacaattttcaaaatatgtatcggcttgactgtttattgtagagtgtagaaaggtttctgtgttcctggctaaagacatccattggtgaaatgtgcttggaaatccaagtgttttctactgaggaaaaaaaaatgttggaaaagctttctcctgcatgcctcttgtgcttagctagaaaggggagctgccagcgtcccagctctgagccactgttcaaaggtgcagctgtgttttaggactaggtacaggcagagtgataaggacatgcctgcttagtgtactcatttatcgagaaagtttagactatgagccacaggggtccctgcctgcagtccctatagcctccccctgccccagtgcctttctcagctggtctgggaatcagggcagcccattgaggggggctcagacaaccctttcttcctggttctgggagtaaggctttctctgcagccacaaggagaacatgggaaccagaaacttctctttggtcagcccttctgagcacacaggtttagcccatgctccaagaatgctcctgagcattgtataagcccctaggactcttagctcatgtgataatgattgcattactatcctgctttaactgacaaaaccacagagaattttgcttgctttcctactctcttcctctgcccaggcctcctccatacgtatacatacacacatgggcatgtatacacacacatgcacaatagacatgcacacacacatgcacacatgggtatacatatacacacatgcacaatagacatgcatatatacatatacatgcacacattgataggcatacacacatgcacagtagacatgcacacatgggtatacatagacacacatgcacagacatgcatacacacacatgcacacataaatgtgcacacacacatacacaaattgcccaagcctgtgcactcctggtacccatgtcccatcctggcccacctctttgtcagcaggactgactccatcactccctcaggttttgcccaccagcaccccccagctttcccccagcagagctaaacagcccctctgaatagacaacaggaccttctagaaacagctgaacccttggacagcagcaggcagattttgatctgctctcatcaggtacaaaaggtggtatccgtattctcttctgagcatgctcagtaagcatatggacatagaatttaacatctctgtggagtgtgtgttttttacatatttgtatgcagtcgaggagggcctgttgtagaattctctccctgtatcttactatactgttaaagaagctgaattctatgttgccaacagatgcgtgaaatgttcctccaggaaaagccattcaagcctgattatttttctaagtaacttcaattaaattgaagaagaaaaaaaaaaaaaaa

SEQ ID NO: 46 - protein, 352 aa, Mus musculus
MKPALLEVMRMNRICRMVLATCFGSFILVIFYFQSMLHPVMRRNPFGVDICCRKGSRSPLQELYNPIQLELSNTAILHQMRRDQVTDTCRANSAMSRKRRVLTPNDLKHLVVDEDHELIYCYVPKVACTNWKRLMMVLSGRGKYSDPMEIPANEAHVSANLKTLNQYSIPEINHRLKSYMKFLFVREPFERLVSAYRNKFTQKYNTSFHKRYGTKIIRRQRKNATQEALRKGDDVKFEEFVAYLIDPHTQREEPFNEHWQTVYSLCHPCHIHYDLVGKYETLEEDSNYVLQLAGVSGYLKFPTYAKSTRTTDEMTTEFFQNISAEHQTQLYEVYKLDFLMFNYSVPNYLKLD

SEQ ID NO: 47 - DNA, 1785 bp, Mus musculus
  CDS (149)..(1408)
ggcgatttcggctgcagaatcagcatcaccagcaacagcagcagcggcggtgactgtggcgggcgctaggtccgtctcctagggaccatgtcccagctgtgcacaaggctgaagtgaagggccaggagtgggcccagcccagggcagcatgaccaagccgcggctcttccggctgtggctggtactagggtcggctctcatgatccttttgatcattgtatattgggacaacgtgggaaccgcccacttctatctgcacacgtctctctccaggccacacatcctagaaccccttcccacccagggattggtggaggagaacgtgttcacatctgacgtggatgagtttttggatactctccttagttctgacgcgaagcacaacgacctttccaggagaaaaactgagcagcccccggcgcccgcccccagcaagccagtcttgagccacatggaggagaacgtgagaggctacgactggtccactcatgatgcccatcagaaccctgaccgggacaggcagcaggccgagaggaggagcctgctgagagacttctgtgccaacgccagcctggcattccccaccaaggaccgctcttttgacgacatccccaactacgaactgaaccacctgatcgtggacgaccgccacggggtcatctactgctacgtgcccaaggtggcctgcaccaactggaagcgagtgatgatcgtgctgagcgagagcctgctggaccggggcagcccctaccgagaccccctggacatcccccgggaacacgtgcacaacaccagcacgcacctcaccttcaacaagttctggcgccgctacggaaagttctcccgtcacctcatgaaggtgaagctgaagaagtacaccaagttcctgttcgtgcgcgacccctttgtgcgcctcatctcagccttccgcagcaagttcgagctggagaacgaagagttttaccgcaagttcgcggtgcccatgctccgactgtacgccaaccacaccagcctgcccgcctcggtgagtgaggctttcagcgccgggctcaaggtctccttcgccaacttcatccagtacctcctagacccacacacggagaagctggcgcctttcaacgagcactggcgacaggtgtaccgcctctgccacccgtgccagatagactatgacttcgtggggaagctggagacgctcgatgaggacgctgcccagctcctgaggttcctcaaggtagactcccagctccacttcccccccagttatcggaacaggacggccagcagctgggaggaagactggtttgccaacatccccctggcatggaggcaacagctctataaactctacgaggccgactttgttctctttggctaccccaagccagaaaacctgctcagggactgagcccccagaagccctcacgctgcccccaacaaattgaatggctgtcccatgaggccgtcctttgaggatgggaccctgtggcctcctgggttctctcctggcttcctttgcttcctggtgtgacaggcagaggattccacgcccccccctcgcatctggagaccgtggtacagccaagaccgaagcacctcactctccagagttttgcgctccccacccccgcccttttgcaatctggatttgtttactccacagcctgtattcatggaacactgtgttaaatactgttttctaagattaatatatttcagatatatttaatacgaaagtgggaggaagctggagtaaagtgtggcgcccgcaaaaaaaaaaaaaaa

SEQ ID NO: 48 - protein, 419 aa, Mus musculus
MTKPRLFRLWLVLGSALMILLIIVYWDNVGTAHFYLHTSLSRPHILEPLPTQGLVEENVFTSDVDEFLDTLLSSDAKHNDLSRRKTEQPPAPAPSKPVLSHMEENVRGYDWSTHDAHQNPDRDRQQAERRSLLRDFCANASLAFPTKDRSFDDIPNYELNHLIVDDRHGVIYCYVPKVACTNWKRVMIVLSESLLDRGSPYRDPLDIPREHVHNTSTHLTFNKFWRRYGKFSRHLMKVKLKKYTKFLFVRDPFVRLISAFRSKFELENEEFYRKFAVPMLRLYANHTSLPASVSEAFSAGLKVSFANFIQYLLDPHTEKLAPFNEHWRQVYRLCHPCQIDYDFVGKLETLDEDAAQLLRFLKVDSQLHFPPSYRNRTASSWEEDWFANIPLAWRQQLYKLYEADFVLFGYPKPENLLRD

SEQ ID NO: 49 - DNA, 1728 bp, Mus musculus
  CDS (1)..(1116)
atgactgtcgcctgccacgcgtgccaggcacagcatgggaagacgctcctgttgcaggcggcccttgccggtggtggcaagtctgggtgctgcactcctgctcctgtgcgccctgcgtcccgggtaaccacaggaaaggatgcccaggacactgaatggcagggctccccaaaagcccttttgggggttccgacatttgaaaataaagctctgggctccagctggttcggtggagtgaggaagagtcccctacagctgttgcgtgacctggaccagggtccacgctccgcgatggccgaggtgcaccagcagcggcgtgagctgctgcgccgggcctgcagccgccacacgcgacgccaacgcctgctgcagccggaggacctgcgtcacgtgctggtggacgacgcgcaccggctgctgtactgctacgtgcctaaggtggcctgcaccaactggaagcgtgtgatgctggcgttgcgcggccgtggggatccaagcgcaatccctgcgcacgaggcgcatgcgcctggcctgctgccctcgctggccgactttgcgccggctgaggtcaactggcggctgcgcgactacctgacctttctcttcgtgcgggagcccttcgagcgcctggcgtcagcctaccgcaacaagctggcgcggccacacagcgcggccttccagcggcgctatggcacacgcatcgtgcgtcgcctacgaccacacgcgcagcccgatgcgctggcccgcggccacgacgtgcgcttcgccgagttcctggcctacctgctcgacccgcgcacgcgccgtcatgagcccttcaacgaacactgggagcgcgcacacgcgctgtgccatccgtgcctagtgcgctatgatgtagtgggcaagtttgagacgatagcagatgatgctgccttcgtgctggacctggtgggtgagcctgggctacgtttccctgctccaccgctgaggccagagaaggaccttacgcgtgagcaggcccggcgccttttccaggacatcagccccttctaccagcgtcgcctctttaacctctataagatggactttctgctcttcaattactctgccccttcctacctgcgactgcaataagggtgttgggtgcaatagagccagtggctgctgtgaccaggaggccaccaggaggtctggaacgaacctggttgtgtggattggagaccttatccagtgggcctgaccaagagtctggccactggtcacactcattccgactgggtagggtacaggttgctttaggtgaccataaccttgtcaggccgtttctgctgttagtttgatgtgtgtctcttcctcccactctgcagatgtcaggcttcttcctaggactccaggtttgtagttctttggtttggtttgagaggccatttctcagtcttgtctgtgtagaacctgtccgtggcatggtgctacaagacagaatgtcatggcttggttcagtgtggcctaaggtcttgtcagcatttactgcttaggagttaacatgagctgcctgccaccccagcagtcacaggatggtgagcactaccactccacatctacctggctgatctacctttgatctcagccttgcaagaggctggatcttcccctgtgtcagcaaaggccaagatgcaatactgtggcagcttttccagctcacttttattttttttgttgtttttttaaataaatatgttttgttac

SEQ ID NO: 50 - protein, 371 aa, Mus musculus
MTVACHACQAQHGKTLLLQAALAGGGKSGCCTPAPVRPASRVTTGKDAQDTEWQGSPKALLGVPTFENKALGSSWFGGVRKSPLQLLRDLDQGPRSAMAEVHQQRRELLRRACSRHTRRQRLLQPEDLRHVLVDDAHRLLYCYVPKVACTNWKRVMLALRGRGDPSAIPAHEAHAPGLLPSLADFAPAEVNWRLRDYLTFLFVREPFERLASAYRNKLARPHSAAFQRRYGTRIVRRLRPHAQPDALARGHDVRFAEFLAYLLDPRTRRHEPFNEHWERAHALCHPCLVRYDVVGKFETIADDAAFVLDLVGEPGLRFPAPPLRPEKDLTREQARRLFQDISPFYQRRLFNLYKMDFLLFNYSAPSYLRLQ

SEQ ID NO: 51 - DNA, 2073 bp, Mus musculus
  CDS (129)..(1259)
ccggccctggtccctgcctgcaccccgggagctggccacccactatccctcccctcccgagacctccagcccctgctgcagtcacctcccctgcagcctcgaggtcggcgaggtctggccgcagcaccatgtttccccgccctctgaccccactggctgccccgaaaagcgcggagaccctgggccgcacgccaaggcgggccccattgggccgggcccgggctgggctcggggggccgcccctgctgctgccgtccatgctgatgttcgctgtaatcgtggcctccagcggactgctgctcatgatcgagcgaggcatcctatcggagatgaaaccccttcccctgcaccctcccagccacaaaggcgcggcctggagcgggacagatcctaagcctagaggcctatccttggatgctggggactcggacttgcaagtgagggaggacatccgaaaccggaccttgagggccgtgtgcggacaaccaggcatgccccgggacccctgggacttgccggtgggacagcggcgcaccctgctgcgccacattctcgtaagtgaccgctaccgcttcctctactgctatgtccccaaagtggcctgctctaactggaaacgtgtgctgaaggtgctggctggcgtcctgaacaacgtggatgtccgcctcaagatggaccaccccagtgacttggtgtttctggcagacctgcggcctgaggagattcgctaccgtctgcagcactacttcaagttcctgtttgtgcgagaccccttggaacgcctcctgtctgcttaccgtaacaagtttggagagatccgagagtaccagcagcgatatggggccgaaattgtcaggcgctacagggctggagctggtcccagccctgcaggggacgatgtcaccttcccagagttcctgagatacctggtggatgaggatcctgaacatatgaatgagcattggatgcctgtgtaccacctgtgccaaccatgtgctgtgcactacgacttcgtgggttcctatgagaggctggaggctgatgccaaccaggtgctggagtgggtgcgggccccaccccatgtccggttcccagctcgccaggcctggtaccggccagccagcccagaaagtctgcattaccacttgtgcaatgttccacgggccctgcttcaagatgtgctacctaagtatatcctggacttctccctctttgcttacccactgcccaatgtcaccaaggaagcctgtcaccaatgacagtaggccagcaccttttggagtttgggtttaatgatatcagctttgggatgtctttcagagaaactcctggctctgggtggcttcctggtttctctaggtgtctccatatctcagtggtaaggactgtccttggaggtccttgtccacagtggctcagaggacagagctagaaaggaggcctgctgctttcactggtgaactgcctctcttaggggcctgtggtatccgtgtctgcagggcaccagtggttattaaagccatatgtttgatcgaaagactgacttcagccccctggctgctgggtctatgcagtccacctggtctgttgtaatttaacctgtggccaaatcccaaatatgacactagccaagcacatgatcatgcctaggaccaatggctgtgaccccctattcacccatcccatggacctcaggactggagtgagctgtggtgccttagaaatgaaatgtgtgcaattctactccagacttttacatttcctcctcttgctaggtctgaatcatttttctaaggaaagagaaacggaagtggggcccttacctcgaagctctaaagcccagcccctcaagcatccaaagacgcctgtgcctgacctcttcctagggctcctggagcatcttcaataagcctcccttccctacaaacctttggagactatgtgagactgtatggcccatatatctggctgtcacttgtctaatgcatttatttaaaatgtgtatattttaataggatccttgtaagggctgacttttaataaagctttttcatatacaaaaaaaaaaaaaaa

SEQ ID NO: 52 - protein, 376 aa, Mus musculus
MFPRPLTPLAAPKSAETLGRTPRRAPLGRARAGLGGPPLLLPSMLMFAVIVASSGLLLMIERGILSEMKPLPLHPPSHKGAAWSGTDPKPRGLSLDAGDSDLQVREDIRNRTLRAVCGQPGMPRDPWDLPVGQRRTLLRHILVSDRYRFLYCYVPKVACSNWKRVLKVLAGVLNNVDVRLKMDHPSDLVFLADLRPEEIRYRLQHYFKFLFVRDPLERLLSAYRNKFGEIREYQQRYGAEIVRRYRAGAGPSPAGDDVTFPEFLRYLVDEDPEHMNEHWMPVYHLCQPCAVHYDFVGSYERLEADANQVLEWVRAPPHVRFPARQAWYRPASPESLHYHLCNVPRALLQDVLPKYILDFSLFAYPLPNVTKEACHQ

SEQ ID NO: 53 - DNA, 6000 bp, Mus musculus
  CDS (496)..(1914)
cttagccactgaaccatctctccagcctctgttttggggtgtttgtttgagacagaatcaaaaggctggtctaaagtacatattgtagctaaagatggcttccaactcctgccttgcctccagagtgttagggtgtgacctttaaatgacctattaaagaaaccccaacaggtgtagagtacacctgtgatcccagctcttaggaggttggacagatggataaagaggtccaggtgtcccggactacatagagagaccctatctcaaaagagaggcgaagacttaggttttgagagtgggcctgaggtctctcttcaaaagttttatagaaagatgtctctgttccctgtctatgaacacggatggcctgagcacctgtctcttcacaggatcagagtgtcccccacctgaagagggctgattgggtcccaagctatgctcctgagctgagtgcctgcagccagtctgaggaactccatggcgccccctctccccatggagaaaggactcgctttgcctcaggatttccgggaccttgtacacagcctaaagattcgaggcagatacgtcttgttcctggcatttgtggtcatagtttttatcttcattgaaaaggaaaataaaatcatatccagggtctccgacaagctgaagcagatccctcattttgtggcagatgccaacagcactgacccagccctgctcttatcggagaatgcatctctcttgtccctgagcgagttggattccaccttttcccatctgcggagccgcctgcacaacctgagcctgcagctgggcgtggagccagcaatggagagccaggaggctggggcagagaagccatcccagcaggctggagcagggacccggcgccacgtgcttctcatggccaccacccgcacgggttcctcgttcgtgggcgagttcttcaaccagcagggcaatatcttctacctcttcgagccactgtggcacatcgagcgcaccgtgttcttccagcagcgaggcgccagcgcggctggttcagccttggtctaccgtgatgtcctcaagcagttgttgctatgcgacctgtatgtgctggagcccttcatcagccctccgcccgaggaccacttgactcagttcctgttccgccggggatccagccgttcactctgcgaggatccggtgtgcacacccttcgtcaagaaggtctttgagaagtaccactgcaggaaccgtcgctgcgggccactcaacgtgaccttggcgggcgaggcctgccgccgcaaggaccacgtggccctcaaggctgtgcgcatccgtcagctggagttcctgcagccgctagttgaggacccgaggttggatctacgagtcattcagctggtgcgcgacccccgggccgtgctggcttcacgcatagtggcctttgcgggcaagtatgagaactggaagaagtggctgtccgaggggcaggaccagctgagcgaggatgaggtgcagcgattgcggggcaactgtgagagcatccgcctgtctgcagagctgggcttgcggcagccagcctggctgcgcggtcgttacatgctggtgcgctatgaggatgtggcacgcaggccactgcagaaggcccgagagatgtacagctttgcgggcatccccttgaccccgcaggtggaggactggatccagaagaacacgcaggcgacacgcgacagcagcgatgtctactccactcagaaaaactcttctgagcagtttgagaagtggcgcttcagcatgcctttcaagctggcacaggtggtacaggctgcctgtggcccgaccatgcacctctttggctacaagttggccagggatgccgcctcactcaccaaccgctccatcagcctgctggaggagcggggcaccttctgggtcacgtagtgggggatgtctgggacccttggaactccttcttgtgaaaggctggccctgcttccctcacacccagcctggcagtgagacatagccctggcagaaagtcaaaatggggagcgatgatgggaacatagcccctggctgtagcggtagggccccctgccagctagactccccagagcagctacagctagggccttgggctcctctgaggacaccctgtctccttagtggtactggtcataaggtgtcctcaccctatgacctgcagtgtcccgagcaggtcaaggtaggttcctgtgtgtggacacacctccctagctctttctcacacagtctacacgaagcctgtaaaggcctgtaggttgtgtgggctggagtccaagtatttaacaaccagaaggggtgtagaccctctgcagcctctcagacctcctactgtattaagtgttaacctcttccgctctgagtcagagaagctgggatctggctgagtcctgggaaggaggaggacagcctagggtgaggaaggggacctttgaagctcctcagggaacagtggctgtgtaccagctcatagaaaatggtttgatcagctgttctagtcactcatgagtatcaatcagcctgtgtagagcaagacacacaaagtgcattgaaaacagacaaggccaggggtgtggctcagcggtaaagcccttgcctctctcatgcccagggccctaggtgtgattcttagcactataaatataagggaaaataagccatacatacgtgtgcagtacagagagagacagaaaagcgtaataaacattcccccctctcataagcacacgctctgcctctgtctctgtctctgtctctctctgtctctgtctctctctgtctctctctctcactctctctctctctctctctctctcacacacacacacacacacacacacacaaacacacacacacacactggtacagaaaagcttcccttctccactctttatgctcaccatgtttttaggatttttgtttgtttgttttgttttgggtttctatatatctaaacaaggagccttaaattacatctttttgggattacgtggcgagggggagcagcagggagcatttgcttctggtaaaaggatagaccagaaaggacttccccttggatgatcggagtcatgcagagcccccagggtcccacatgttctgtgtgaacttcatgtggaatgactcacagaagtgactaccttaggcgctcgctctgtagctcaatgtgcgtagagtttgtctattgtataggaagccctgggttctgtgccctgccccacaccatataggtatgatggcgtgtacccatatcccagcattcaggaaatcaaaggttggcggtcaaaagttcaaggtgcagctatgtatcttttgagatgtccgagtctataagctccctctactgagagctgctagtattccttctcaagaacaggttatttgaaaatctcttcttgtaagccccaacatttgggggaggggaggatgggggaactcttgactgagcagctaggtttccccaagcagaagttcttctctcccagtgcaagctgaggtttagtgccccggctagcctcggaactggctcagtgggggttggcacgctgacttcctggacaattgctgcatctgacactgtgagcgcccaggacaccggatgtgtagcatggatgtgggcctagcacaggtgtggaagaggacagtcaggacactcactgttggtgtttgttatccacctgttcagccgccgggtcccacacataggtcatctttcgtcactcaagccggcctctgtttttgattttaacaatccaagagcaagtgtgtaggggacaaagagacaccagctcttcatacttgatggagaccgggacaggatgctgcaggcaggctcgggagtgcattatcgtttcttctgagctcttcatccaggccatattcccttttctgcatcgcatctggggtgggaggggctgagctgggagtttccgtccttcttccctgtgggggtgggagatggggctcaaggtctccatctctcggctccctgaggacagaacccccacagtggacctttgggccttctcaggacattgacaatgttgtgtgcactgcaagttgactttatttattttgtaggaaaaagagatggtattctttagcaggatctgaaactgtaccctagtcaagaagtacaattaataacattattattaataacaattgggatgattcaaaggtcacacacacaccagaccacccaagaaccatgtaggagatgaggcggagccaggtcatggaagcccagaatgctacagagattctgtgggtctattattgatcagaaaatacaacatgggggggccttttctatggcataactcaggtgtctgcaaagacagagcagctcaggtgggtggataggcaggcagatggagccttgaatcacatcccctgaggctcagccagccagtgcgaatcctcaaggttcggaaagggacagtgtagcggcaaactactgggcatcttctctgatgcttagaggctcttaccaaggagcttgatggagaaagtggccatgttggtggcatgggacaactgctcacccaacttcaaagatcaaagggcacccaggtggcctatgacagtgacactctttcctaccattaggggtgtctgcccccactgaaagccatgaaatttctggccctaagggggtaggaaggacttaggagtagcagatggtttgatcccatcccccccacacacacacacacatccctcagctgtcttccacattagagccacttcagttgtccatggacttgtccctttgagacatcctggattttgaaggatagaaacatccccaagatggtctcgtgttaatcccacaacagaggcagaagggtcatactgagagagagagagagagagagagagagagagagaggagtgggggccagaccctcaagaagccaagtgggtctggtcaacctgtgcacatgagaaggagggaacatcactaaaatcagggcctgggctggtgtgttgttggtgagatcccgtggagtggctggctagatatggatgagttttctgagcatgctcacacacccccaacttcacattcttagaaatagcacaaccataatgccttacctcaaaaggatgaggcaaagcttgcaattaattcccagtgtctgaagacctccgggatcctttgagctgtgtgtccttgtcagtacatggggacaggctccttaggtattcctgacatagaggtaaggtgctgcccttgctgccttgcataagctgtgacaagcttcttactgggcacatagaacggctctgtcatctgcttccactaaatttgggcttggactttgcctcctgccaaatctccatctctgctggatagtctagtccctagggtctaaccaccaccctccacttcctggtgggtcctacaagcgctgtctttgtgccagtacccggatggtgtcctgcctcatggctaaatggtacaggacatcttcccagactgagtggtcatggcatgtgcatgtatgttcatgagtacatgcatggtgtgtgggggtggatgggtgcattctgttctgcttctggtgcagctacacagccacaacctctttctgtcattgaccttcttggtcttcttgtagccacagataatcttccaatgcccgattctgctgttctcatctgagagctgacaacccagcgctcagagtagagttcatgacctaggaaacccttctcctggtagcttactgaacttatttaattaaaaacgaacatataggggttggagagatggttcattggttaagagcaccggctgcttttgcaaaggacctgagttcaaatcccagcaaccatatggtggcttacaaccatctgtaatgagttctggtgccctcttctggcatgcaggtgtacatgcagatagagcactcctatgcataaaataaataaatcttttaaataaataaataaatgaacatgaaaaaaaaaaaaaaa

SEQ ID NO: 54 - protein, 472 aa, Mus musculus
MEKGLALPQDFRDLVHSLKIRGRYVLFLAFVVIVFIFIEKENKIISRVSDKLKQIPHFVADANSTDPALLLSENASLLSLSELDSTFSHLRSRLHNLSLQLGVEPAMESQEAGAEKPSQQAGAGTRRHVLLMATTRTGSSFVGEFFNQQGNIFYLFEPLWHIERTVFFQQRGASAAGSALVYRDVLKQLLLCDLYVLEPFISPPPEDHLTQFLFRRGSSRSLCEDPVCTPFVKKVFEKYHCRNRRCGPLNVTLAGEACRRKDHVALKAVRIRQLEFLQPLVEDPRLDLRVIQLVRDPRAVLASRIVAFAGKYENWKKWLSEGQDQLSEDEVQRLRGNCESIRLSAELGLRQPAWLRGRYMLVRYEDVARRPLQKAREMYSFAGIPLTPQVEDWIQKNTQATRDSSDVYSTQKNSSEQFEKWRFSMPFKLAQVVQAACGPTMHLFGYKLARDAASLTNRSISLLEERGTFWVT

SEQ ID NO: 55 - DNA, 2080 bp, Mus musculus
  CDS (147)..(1601)
caccgcccggctgctccactccgctccacccaacattgagggagacccgaagaggccggagccgaggactttgggctgggttttctggacagaaccagcaggcgcctactctgctctgggtggggagagtgaggattcggtgaactatgaagggccggcggcggcggcgccgaaagtattgcaagttcacgctgctcttggcgctgtacacgcttttgctacttcttgtcccctctgtactggacagccacagcgagcaggacaagggcaggaactgccccggcctgcagcgcagcttgggtgtgtggagcctggaggcggcggcggccggggaacgtgagcagggcgctgaggtgcggtccctggccgaaggaaacccggatcgatcccccgggtcccccggcaacctcagcgccgtcggtgaggcggtgacccaggaaaagcaacacatctatgtgcatgccacctggcgcaccggctcgtccttcttgggcgaactcttcaaccagcacccggacgttttctacttgtacgaccccatgtggcatctgtggcaggcactgtatccgggcgacgcggagagcctgcagggcgcactaagagacatgctgcgctccctcttccgctgtgatttctctgtgctgcgcctgtacgcgcagcctggggaccctggggagcgagcaccggactcggccaacctcaccacggccatgcttttccgctggcggaccaacaaggtcatctgctcgccgcctctgtgccccgccgcgccccgggcacgcgcggacgtgggactcgtcgaggacaaagcctgcgaaagtacctgcccgcccgtttcgctccgcgccctggaggccgagtgccgcaagtacccggtggtggtcatcaaagacgtgcggctactggacctgggagtgctggtccctctgctgcgtgacccaggcctcaacctaaaggtggtgcaactcttccgagaccctcgggccgtgcacaactcgcgcctcaagtcgaggcagggactgctgcgcgaaagcatccaggtgctgcgcacgcgccagaggggcgaccacttccaccgggtgctgctggcgcatggagtggatgcccgtccgggaggccaggcccgggctctgccctcggcgccacgcgctgatttcttcttaaccagcgcgcttgaggtgatctgtgaagcgtggcttcgcgacctgctattcacccgcggcgcgcccgcctggctgaggcgtcgctacctgcggctgcgttatgaggacctggtgtggcagccccaagcccagctgcgccgcctgctgcgcttctctgggttgcggacactcgccgcgcttgatgccttcgcattcaatatgacgcggggctcggcctacggcgccgatcgtcccttccacttgtctgcgcgggacgcccgagaggctgtgcacgcctggcgcgaacgtctgagccaagagcaggtgcgccaagtggaaaccgcctgcgcccctgccatgcgtctgcttgcctaccctcgaagtggggacgaacgcgacaggaagaccgtcagggaaggggagacaccactggagaccaaggccaattgggctgtgtaataccctgatccctgaaccctgccccggggcgtattcaggtcagtggccataaaaaggtgaactcagcatgctgcccccgcactggagaggctgcacggtggaggcgatctatcacactgtgagacactgggactgatttggtatcaactgctgtgccattctcctggtcaggagcatcacaagctgttaagtaatgacagacaccttggctgagatgaagtttccagaaaggaagtaacagtgcaatgtggatatttgtgaccacaacataggaaaagctgtacttcccaggctgaacttggctcagcttgagccatttcaacaaggcatcctcacaataatgaagagatgtgatctggtttcctttcacatcagccaagatgtctggacaaaaccatcaatgtgaataagggccaagtgcagttgtgtctctcttgattaaattacttcatattaaataaaaaaaaaaaaaaaaaaaa

SEQ ID NO: 56 - protein, 484 aa, Mus musculus
MKGRRRRRRKYCKFTLLLALYTLLLLLVPSVLDSHSEQDKGRNCPGLQRSLGVWSLEAAAAGEREQGAEVRSLAEGNPDRSPGSPGNLSAVGEAVTQEKQHIYVHATWRTGSSFLGELFNQHPDVFYLYDPMWHLWQALYPGDAESLQGALRDMLRSLFRCDFSVLRLYAQPGDPGERAPDSANLTTAMLFRWRTNKVICSPPLCPAAPRARADVGLVEDKACESTCPPVSLRALEAECRKYPVVVIKDVRLLDLGVLVPLLRDPGLNLKVVQLFRDPRAVHNSRLKSRQGLLRESIQVLRTRQRGDHFHRVLLAHGVDARPGGQARALPSAPRADFFLTSALEVICEAWLRDLLFTRGAPAWLRRRYLRLRYEDLVWQPQAQLRRLLRFSGLRTLAALDAFAFNMTRGSAYGADRPFHLSARDAREAVHAWRERLSQEQVRQVETACAPAMRLLAYPRSGDERDRKTVREGETPLETKANWAV

SEQ ID NO: 57 - DNA, 512 bp, Mus musculus
atgaaaggcatcttatggatgtgctcaaccttattactaacccatgcactacatcaagccaaaatggaaaccagcccacctgttaaaggctctctgtctggaaaagtggtcctaccttgtcatctttcaaccttacctaccttaccacccaattacaacacgagtgaatttctcagaatcaaatggtctaagatggaagtggacaaaaatggaaaagatataaaggagacgactgtcttggtggcccagaacggaaatatcaagattggtcaggactacaaggggcgagtgtccgtgcctacacatcccgatgatgtaggtgatgcttccctcaccatggtcaaactccgggctagtgatgcaggcgtctaccgatgtgatgtcatgtatgggattgaagacactcaggacaccatgtcactggctgtggatggtgttgtgtttcactacagggcagccaccagcaggtacactctgaactttgccgctgctcaacaggcttgtttggatat

SEQ ID NO: 58 - DNA, 536 bp, Mus musculus
acaaagccatgaagccggcgctgctggaagtgatgaggatgaacagaatttgccggatggtgctggccacttgcttcggatcctttatcttggtcatcttctatttccaaagtatgttgcacccagtcatgcggaggaaccccttcggtgtggacatctgctgccggaagggatcgagaagtcccctgcaggagctctacaatcccatccagctggagctatccaacactgccatcctgcaccagatgagacgggaccaggtgacagacacctgccgggccaacagtgccatgagccgcaagcgcagggtgctgacccccaacgacctgaagcacctggtggtggatgaggaccacgaactcatctactgctatgtgcccaaggtagcgtgcaccaactggaagaggctcatgatggtcctgagtggccggggcaagtacagcgatcccatggagatcccagccaacgaagcccacgtgtcggccaacctgaagacccttaaccagtacagcatcccagagatcaaccaccgcttg

SEQ ID NO: 59 - DNA, 660 bp, Mus musculus
gccaggagtgggcccagcccagggcagcatgaccaagccgcggctcttccggctgtggctggtactagggtcggctctcatgatccttttgatcattgtatattgggacaacgtgggaaccgcccacttctatctgcacacgtctctctccaggccacacatcctagaaccccttcccacccagggattggtggaggagaacgtgttcacatctgacgtggatgagtttttggatactctccttagttctgacgcgaagcacaacgacctttccaggagaaaaactgagcagcccccggcgcccgcccccagcaagccagtcttgagccacatggaggagaacgtgagaggctacgactggtccactcatgatgcccatcagaaccctgaccgggacaggcagcaggccgagaggaggagcctgctgagagacttctgtgccaacgccagcctggcattccccaccaaggaccgctcttttgacgacatccccaactacgaactgaaccacctgatcgtggacgaccgccacggggtcatctactgctacgtgcccaaggtggcctgcaccaactggaagcgagtgatgatcgtgctgagcgagagcctgctggaccggggcagcccctaccgagaccccctggacatcccccgggaaca

SEQ ID NO: 60 - DNA, 600 bp, Mus musculus
atgactgtcgcctgccacgcgtgccaggcacagcatgggaagacgctcctgttgcaggcggcccttgccggtggtggcaagtctgggtgctgcactcctgctcctgtgcgccctgcgtcccgggtaaccacaggaaaggatgcccaggacactgaatggcagggctccccaaaagcccttttgggggttccgacatttgaaaataaagctctgggctccagctggttcggtggagtgaggaagagtcccctacagctgttgcgtgacctggaccagatgtttggcagctgtgagctatgggtagtcagtggggagcggcagaaagtgggtccacgctccgcgatggccgaggtgcaccagcagcggcgtgagctgctgcgccgggcctgcagccgccacacgcgacgccaacgcctgctgcagccggaggacctgcgtcacgtgctggtggacgacgcgcaccggctgctgtactgctacgtgcctaaggtggcctgcaccaactggaagcgtgtgatgctggcgttgcgcggccgtggggatccaagcgcaatccctgcgcacgaggcgcatgcgcctggcctgctgccctcgctggcc

SEQ ID NO: 61 - DNA, 600 bp, Mus musculus
gcgccccctctccccatggagaaaggactcgctttgcctcaggatttccgggaccttgtacacagcctaaagattcgaggcagatacgtcttgttcctggcatttgtggtcatagtttttatcttcattgaaaaggaaaataaaatcatatccagggtctccgacaagctgaagcagatccctcattttgtggcagatgccaacagcactgacccagccctgctcttatcggagaatgcatctctcttgtccctgagcgagttggattccaccttttcccatctgcggagccgcctgcacaacctgagcctgcagctgggcgtggagccagcaatggagagccaggaggctggggcagagaagccatcccagcaggctggagcagggacccggcgccacgtgcttctcatggccaccacccgcacgggttcctcgttcgtgggcgagttcttcaaccagcagggcaatatcttctacctcttcgagccactgtggcacatcgagcgcaccgtgttcttccagcagcgaggcgccagcgcggctggttcagccttggtctaccgtgatgtcctcaagcagttgttgctatgcgacctgtat

SEQ ID NO: 62 - DNA, 720 bp, Mus musculus
tggggagagtgaggattcggtgaactatgaagggccggcggcggcggcgccgagagtattgcaagttcacgctgctcttggcgctgtacacgcttttgctacttcttgtcccctctgtactggacagccacagcgagcaggacaagggcaggaactgccccggcctgcagcgcagcttgggtgtgtggagcctggaggcggcggcggccggggaacgtgagcagggcgctgaggtgcggtccctggccgaaggaaacccggatcgatcccccgggtcccccggcaacctcagcgccgtcggtgaggcggtgacccaggaaaagcaacacatctatgtgcatgccacctggcgcaccggctcgtccttcttgggcgaactcttcaaccagcacccggacgttttctacttgtacgagcccatgtggcatctgtggcaggcactgtatccgggcgacgcggagagcctgcagggcgcactaagagacatgctgcgctccctcttccgctgtgatttctctgtgctgcgcctgtacgcgcagcctggggaccctggggagcgagcaccggactcggccaacctcaccacggccatgcttttccgctggcggaccaacaaggtcatctgctcgccgcctctgtgccccgccgcgccccgggcacgcgcggacgtgggactcgtcgaggacaaagcctgcgaaagtacctgcccgcccgtttcgc

SEQ ID NO: 63 - DNA, 600 bp, Mus musculus
accccacaactcggaacgatgcggctagcctgcatgttctcgtccatcctgctgtttggagctgcgggcctgctcctcttcatcagcctccaggaccctatagagctcagcccccagcaagttccaggtataaagttcagcatcaggccccagcaaccccagcatgatagccacttgaggatatccacagaaaagggcacacgagattcacccagcgggtcgccaagaggcctccagctgcaagcgcctgaccaacctcgacctcacccgaaggcagcgggatctcctttgcgcctccggcagcgcaggcggagactgctcatcaaaaagatgccagccgcagggactaaccaaggcaacaactcgtccgaaacctttatccagccgagaccccgcaccatggacagtcgttgggtcagcctgcaccagacccaacaggagcgcaagcgtgtgatgcgagaagcctgcgctaaatacagggccagcagcagccgcagagctgtcactccccgccacgtctcccgcatcttcgtggaggaccgccaccgtgtactgtactgtgaagtacccaaggcaggctgctccaactggaagagggtg

SEQ ID NO: 64 - DNA, 735 bp, Mus musculus
atatagtatctaggatatatgtagaagacaaacacaaaatcctgtactgtgaagtaccaaaagctggctgctctaattggaaaagaattctgatggtcctaaatggattggcttcctctgcatacaatatctcccatgatactgtgcactatgggaagcatctgaaaacactggatagttttgacttaaaaggagtacacatgcgtttgaatacatataccaaagctgtgtttgttagagatcccatggaaagattagtctccgcatttagggataaatttgagcatcccaatagttactaccatccggtgtttggaaaggcaattatcaagaaatatcgaccaaatgcctctgcagaagcattaaataatggatctggagtcaaattcaaagaattcgcctactatttgctggatgctcaccgtccagtaggaatggatattcactgggaaagagtcagcaaactgtgttatccgtgtttgatcaactatgactttgtagggaagtttgagaccttaggagaggatgccaattactttctacagttgattggtgctccaaaagagttgacatttccaaactttaaggataggcactcctctgatgaaagaaccaatgcccacgtggtaaggcagtatttaaaggacctgagcacagccgaaagacagctcatctatgacttctatcacttggactatttgatgtttaattacacaactccacatttgtaatttg

SEQ ID NO: 65 - DNA, 574 bp, Mus musculus
ccagaagccaagctcattgttatgctcagggatcctgtggagaggttgtactcagactatctctacttcgcaagttccaataaatctgcagatgacttccacgagaaagtgacggaggctctgcagctgtttgaaaattgcatgctggattattcactgcgcgcctgcgtctacaacaacaccctgaacaatgccatgcccgtgaggctccaggttggcctgtatgctgtgtacctcctggattggctgactgtcttcagtaaggagcagttcctcattctccgtctggaagaccacgcatccaatgtcaagtataccatgcacaaggtcttccagtttctaaacctggggcctctgagtgagaagcaggaagctctgatgaccaagagccctgcatccaacacacggcgtcctgaggatcgtagtctgggacccatgtggcccatcacccagaagattctgcgtgaattctatgggcctttcaacacaaggctggcacaggtcttggatgacgaagcatttgcctggaagacaacgtgagagctgagtcccttctgcagaagctgggcccact

SEQ ID NO: 66 - DNA, 1430 bp, Mus sp.
tgcgacttcatcaagtcggagccaaagaaatcttgaaagatgggaatgctagcaagagttgccctgggacttatcatcattgatgctgtattggctgcaccaactacagagctctttaactatgactcagaagtgtatgatgccatcttggaggacacagggaccttttataactatgaacacattcctgacaaccatgtagagaacgagaaagtgtctgagaggctttctgggaacagagagctcctgaccccaggcccacagctaggggacaaccaggatgaagacaaggatgaagaatctactcccaggctgatcgatggctcttccccacaggagccagaattcccggggcttctgggtccacacaccaacgaagactttccaacttgtctcctgtgtacttgtataagtaccactgtatactgtgatgaccatgaactggacgctattcctccactgcccaagaagactacatatttctattcacgttttaacagaattaaaaagatcaacaaaaatgactttgcaagcttaaatgatttaaaaaggatcgatctgacatcaaatttaatatcggagattgatgaagatgcattccgaaagctgcctcacctccaagagctggttctgcgtgacaataagataaagcagcttccagaattgccaaacactttgacatttattgatatcagcaacaatagactgggaagaaaaggcattaaacaagaagcttttaaagatatgtatgatctccaccatctctatatcaccgataacagcttggaccacatccctctaccactcccagaaagtctgcgggctcttcacctccagaataatgacattctggagatgcatgaagatactttctgcaatgttaaaaatttaacttatgttcgtaaggcattagaggatattcgactggatggaaaccctatcaacctcagcagaactcctcaagcctacatgtgtctacctcgtttgcccattgggagttttatctaatgttaggatgctaggcaaatatcagaattgtgatgctttctgtagccaagagaagcatatatctcttcagaagcaaactcagtatatttgagatgcgtttaaaacatgtggcacaatgatataaacaataagctaaaggtgttgagataaaataagagttcaataatttaattaaacatgattaggtataaattaacaaaatatatagacagtaagttaaataaaatgtatgccaactagtcatcaaatctcatcaagaattgtaggtgttcatgtcaagttaaatatgtaagaaataaaatattggtaatgaatactttagatacgttgctaagatgtagacattttaattaaacttctctagtctttattttcactcatgtatgtttcccacctgattcagttaagagcagaaaaaaata

SEQ ID NO: 67 - protein, 20 aa, Mus musculus
DRARSCAIVEDDGLQSAFTA

SEQ ID NO: 68 - DNA, 9120 bp, Mus musculus
tgtatatctgagtatctgtgtatgtgcatatatgtgtgtctatgtgtatgtgtgggtttgtgcatgtgtgtctgtgtgtacatgtctgtgtgtctctgtgtgtgtctgtgtatgtgtgtttacatgtgtgtgtgtgtttctgtgtggacgcatgtgtatgtgtctccgtgtgcctgcatgtgtgtttgtgtatgtgtgtaggcatgagtacatcatggtgtacatgtagaaattgagagagactcttgggtgtggttctcatctcccaccatatctgaagtaaatgtcttattcacaccactgtgcacatcagaaagctagccttgacctccaaggaactctgtctctgtctcctgtcttattgtaggtacctggggtttataagcctatgctgctgcatatggcttcacttgggttctggggatttgaacttaggtcctcacagttttagggcaagcattttattcactgaacaatctcccatgatccctagagtttattgtaggcaacttaacctttagtcgtcattgagatttagcattgatttcagttccagttgtattctgatgttgagcgctacagcagaggtacccagggactgctgattttctgtggccatgaatcctctagtgattaataaatttcatcataatgttctgtttatttctaaatgtccagacaactctaatttcaaattaatctcatcctaaacagcacaggataaaagttacagattgtcctatcttcattcatgtgatgcctcaaatcagatgtttaatcctttagtcagttatttgtgaaacggaacctaattacacacactcgcatgcgcgtgcgcgcacacacacacacacacacacacacacacacacacacacacacttgtagatttgcccacacagctgcttaagcaatttggagcaaatgtagaaatattatgaaatttcacataaaaatgggttttcctctgtcttcttgagaaatgtgtgagtttcaatatgacagtaatcttctggtgttggactataaccacttgaacccctgaggtactaagtgtgtacctactacgtgcctggattccatcatttccctacttatataagcttctccatcctgtagatcagcactgtgtaatggagtaacacaagtcattgaatcatgcaatcatttaatcaatttaattggcacttctgataagagaagtgggcacatatttagttgtgtttgcagcctgtgctatgtcatttttctctatatatactttaagatttttttcttttttccatttacggggggggggttattccatttaatttacatgcttaatgacatataatttgcatatcaggtgttcctttctttagagttaacttcttttaggaccattgtttataatggatgtatatcatccctaaattaaatcagccattgtctacttaacacaccagtttctctgatgtccctctggctcttcctcctctgaggttctagttataggaaagtctggcattttgatgtttttccatggcttcgtgggctctgatcatccacttacagtgtactttcatctcttgtctacaaactgcatatcatctattgatctgcgttcaaggctactacattttcatgcaattccactttgctgttaaaccttttctatgatccgtttttatacttataaacaatccaagtgttgtactctttgggtataaatgttctgtatttcacaggtcttaacttcttttcagactatacaaagaaagattttctattttttttatttgttgtatgcattttatgcatttagcactgggatttccataacaggcagttaagatcattgaatacatactttgtgtggtaaatataatacggcttatttgattctcccccttaaaatgctcccatcttctcagtcatgtcaggttgctatggagtccaccctgagcaccatgagtgtgatgctgtacagacactcctgatatgccttctgttatttgaactccacctgcaagttttcattcaagcatagctcctaatcccattcagtgtgcacttttcttaattgatttgctggtagagagctatatgtaagcatggcttagcatcaaattgaactttctcctctccctagcttctttccagtatattcttcctcaccttcatcagattttgaggtcttgagatctaaaccttcatgttacaatttgcagagataccaggatctctatcagacttgatgtgtctccatgtaattctagtatcagcctttcataagataattgcaaaatatagaaccccccctctctccttttgactattacagctttgtttctctcccaagttaatacagatgtatgcaaatcaactaagaaaagcaaacactgaatgggataaggagctatactcaaaagaaagcttgcagttggagttcaaataacacaagtggaaccaagagtatctgcacagcatcagactcactgtgctcaggatggaatccatagcaacctcccataactgagaggatgtgagccccttttaaagggaaaatcaaataagcagtattatattaaccacatgatgtgtatattcaatgtagattgggctgcaccgaagttatgattttagtaatgagtatttctccttgggttttcttctggaccaccctggttttccttttaaaaatagtaaaatagcagcaatgactgcttctcttaaaatctgaacaacacagagcacttcagcatcctttcaacctatctaaatccattgtagacctgcactgacttgctgtgagctattgtgtcacaactattattgtagctaaacaccttgaattttcaaagctgctctggactgtgtcttcttttagaattaaacttgctaaagatattggactctgagatgcttattcaccactttctataaaagagtaaggatgacaatgctttgagtagaagaagattgcatacgagggtaccagcatgtcattatgtccccagcagtcagtaaagtaaggatccagtgatggtagagttattatgtccattatggaactaatgtaaatgtttatcaatgactagaatggctgtaatcaccaatccttagacaagtgtgagtaacatcatgctcttttcttataataatagatagtcacacacaccatggtcttaaacattacattttgatttttctctttgtgtctgtcttttcgtacctctactccacttacctgagccttcagctagggctcctccaagacggcagtcacagtactagtgaggataattctcatatggagacctgaagcagaataaccttcttccaaggaaaccaaaatcctagtcaacatcttctccactctttggttatttatttttatttctatttctattttgccatttaagaatttttaaactcttttctgttttcttatatatattcttttttttacaacttttgatattctttatcaaatgatttatcccattgcctttcctcctgcagctatctgtgagttttagcatggatatttagaatacaattggaaattatgaaggtcgaataaaagttctctaagagccaggacctcactagcctagttagttgcttagtttctaatataaggcatggtttatctcctactgtatggcccttaaatccactagacatctgttggcttccaacagcatacaattgctgctattgtatgtttaggtatatatttttatgaaacactatattctgattggtgttcattcttcctatgaaggggaagattttattgcctgctcaaattcaagtcagtcctttttgagtagacaaaggttgtccaatcttgctaaacccttttcttttttgcttggggaatttctttagggaaaattctcaaccatgaaccaagcctagagattgataatagctgaaggcagagtagccagtcttagtaagggatgagagaaagactgaccaaagactgaataggaaatggttgatagcattcattgggactagacaataaagttagctaccccaacatgggcaaatggagaaaatggggagagtcatatggatatgtagggaggagaagaaaggaaggggtgagtttgaagtatggaagagcctttaaagcatttgtaagatttgtaagaaaaagcttttgagagacagagtattacctgccttcaaatcccttcaattactgtccctacaagacatacctgtgctcagctgctcttggaccacaagggggcctaagagcatgtttgtgtccctgtctttatacaggcttctcatctgtatttctcaaacgtaaatacaaatgggggagataacaaatgatatttctaagtggtacacaggtatgagaactgcacacaatctatttatatgtataatggccagagctctcagcagggaacagaggaagcctaggtaactcctgctccagagcatgtctgtatggagacctcagttcagcaactatgaaatctgtgttcaaagggtcatcactgggaacagtttctggaatagcagacatattcattctctctctctctctctctctctctctctctctctctctctctctctctctctctctctgtctgtctctgtctctctctgtctgtctgtctgtctctcccttctgtcaggggtatgcatacatgggctgggaattctatcttccctgcctgctgtctttggtgtctccctaatgtggtgccatgtttacttaacctgtctgaaatggaggataacctctggtctgataactgtcaagttactgaaatgtcagggaattgtgaatgaacccctctgaatgctgggcagatgttttgagcacgtgggtatccatagatttttttttcagcaggatttccttctttaatatattgtgggaacgaaacattgaagtttccaacactaagaaaagcaatgctctgggtttgcaaagaagtctccaaagctgtgggagaggcaatggctttgcctcacaaaggtgatcctgatttggattgggtgttcctcagtctccctcatcctttgtttgtggaccaaacctctgatcaaataatcagtatacagacaagtactgactgactcctgtcactgtttcccatgtaaattctcaaccatgtgtgtcagaattgtttgagtcatcaaagttccgtgatgctcaggatggtgaataccagaaactgaagtctgaaatccaatttggcctttctcgggcactgtaaactgagtgcaaatgtgcccagttccctcgtggcccatttatagcacaataacacagtgcttgcttacaaaatctagtcccgattaaagtatttatttaaagttgctttagacaaatatatttctaaatggaaggatgaaacagaaagaatattccaactgtacagtctatattttgtttctaaaaatttttattagaattctgacttttagaaaacaaatagatattcaaaaactacttctttaattaaataataacatcattatagacattctccagccatttagaagtagcatgattacaaagcattcatgttccctttgttttttgaaacagaagtatttatatgacaaataaaaagcaaaaatctcatatatgtgatagctatagatgcattgataggaattaaatttaatcaaaaagaacttttaatggcaataatgtggctactaacttcattgtttgtaataatgctgcaaatgtttaatgaaaactgttactaacaatacaggagaagattacagatatgattaggtcgtgttgatagaaatggaaagagaaaggtcactcactacctcagccacagaacttgaggaatgtgtgagggtttttgaggaaaaaagagaacaatgtaacatcacagcaacatcttaatctgaaagtagacgacaaaggtttcagagtaggggatcagaaatttaagtatgcagagaagcaaggcatatacggcttagacaatgtatgaagaacagcagcagacagcaagtgaaagaaatcacagtccacatggaaatcacacagaccacagtgtgcacacagagacagaaatcagcaagggacacacggcttccagttatgaaagttagacagcaaaatttaagttgtcccacctaaaacatgatgtatcgtctctcaaatggggaaggggaaggagcagcaaacatctggatatgtctaaaacctcacagagaacatctgggatctttgcatttctccctctgactttctcaatatgttagctacttctagatagtttcaataatggtctctaattgactatggagagacaggtctcatgtatgtgaacatccttaaccaggagatggaatgtgtttccttgtcactcattttctaaggaagtgaattccttctgcattgcccaggattggatgttacgtgaaactaaaaatcattgattaaaaagaaatgtcattaccacagttgggttgaatcaatcaagacttatcctagttacaggcatcacctccccttaagcacaaatactgagaacagctaagctcactttgaaatctgggatgaggaggctttaggtcaagcatctgacaatgtgtgtgtgtatgtgtgtgtgtgtgtgtgtgtgtagtatgattatatgtatatatgtagatgtgtgtatatatttatatgtatatatgtatatgtgagtatgtatatatgtatatgtgtgtatgtgtatgagtgtgtgtatgtatgactatgagtgtgtatgtattgtgcaagtgtgtgtatatatgtgtgtatatgtttatgtatatgtgtattgtatgggtgtgaatgtattatatatgtgtgtaggtacatatgtgtatatgtttatgcgtgtatatgtgtatgcattatatgtatgtgtgtatgtgtgtatgttttcatgtgtatgtagcatatgtatatgcatgtgtgtgtctgtgtgtgtaagcatgtatttactaagaaaaaatcagtttctctagctaatgagtcattgaagtgaagaaaccttacgctatcacattactatgtttccagcataaattgcaatatcagtaagacattccccatcataacttaatggacacttagattaggtcatgcagcagtaaattatgaaagcgtctatgctgagctgaggtgggaaatcgtctttcctgcctccttcctcctgttctttctcctaaaggccctcatcctctttctccccttcctttcttttaaaatattctatcattgatccaagctaactaacccttcccttgctcacatgtgacctgcccagaggaatctatttatagtcaacaataacaggccaaggacatgcgaacaaatgactttacttagccccacttggtgaatctattgggtgaataggtaaggggttatttataggagcttgactgactggcacagctgtatctctgaaaagtcccccaaagcatcatgggtgggaactaaggaaagccggaaatgctggagctggaatcccaacctgcagtggctacatgatgaatgtgtctcctttcctactaaatatttaagactctgggcaggctggttagtcttgtaaaagtcttgtgggtttctggagtcctgggaggctcactgtccctgcagaagcttgttattattgtatatgaaagaaagtgctcaacactgagtcacaaaagaagcccccgagaatcggtggcagaggtggaaatagaagaatccgatgcagttcccgaggcaagagttgatcctctccatggtgacaagaaaacaagagccacagaagcctgaaggctaagactatgacctgtgaggttctcctctcctaggacttgaatgcacttaactctgagggctagcagggcagagattcaggagcgttctcagaggttgtcccttctccttgtatgattctaggagaagctcagtgcataggcagaagttgcaaactctaacacattaacccacatcctgaattgttttatatcaattgttttataactacatgttttaacttttgttttggtttttgaattttgtttgtttgtttgttttctatttttcatatgtgtgcatgtgagccagaagacaggctaagggactctgttctctctttccagcatgtgggtttcagggatcaaagttaggtcccaaggcttcactgggcaacgggacccttagcctctgagtcatcttcttaatctttttctttgaaactttttcattacattgtgtcagtgcacacacacacacacacacacatgcatatacatccacatgcatatacatccacatgccatatgtcatggcacatgtgtggaaagaaatgggtgccttgaggaaatcagttttcttcttccatcacgtggatcttatgacttcaggtaacccatctctgctccccaagcatggctacaagcatttacctgccaagtcatctccctggctcccacattaatttataataatacaccctttcagcctagagagatgactcagtggttaagaactctggctgtgcttcagaaggttctgagttcaattcccagcagctataaggtggctcacaactatctctaatgttgttttctgtcctcttctggcatgcacttgtatgtgcagatagagtacttatatgcataaataaatataaatacattaataatcttttaaaaagaaggaaagattaaaagaacaatacaccctttcttttaagtaagaaaacatgcttagtatatagaataaatgctggtgttgaaaggtgaaattcagaagaaattataggttggaaatatttgaactcttttgtgtcaaaccaaatggtaaagatgatgtgataataaggtaaggttttcttcctacacatatacttgtttaataaccagtataagactatgagtgaacataactaaaaatacatgaaaaaaaactattttaaaaaaacatcactttcagatgactctggtgaaactcactgataaccagcaatctctttgatagaccagagtgtccagtctgaagttaattcttttgctttattctcaaacaatgttgcagaagtatgaaccatagtatcaagatacactgtccacatatgcacctggcctgacttgtgt

(19) United States
(12) Patent Application Publication
(10) Pub. No.: US 2009/0060892 A1
(43) Pub. Date: Mar. 5, 2009

(54) AGENTS FOR SUPPRESSING HEPATIC FIBROSIS

(75) Inventor: Hiroyuki Yoneyama, Tokyo (JP)

Correspondence Address: BOZICEVIC, FIELD & FRANCIS LLP, 1900 UNIVERSITY AVENUE, SUITE 200, EAST PALO ALTO, CA, 94303, US

(73) Assignee: Stelic Institute of Regenerative Medicine, Stelic Institute & Company, Tokyo (JP)

(21) Appl. No.: 11/912,061

(22) PCT Filed: Sep. 27, 2006
(86) PCT No.: PCT/JP2006/319128

§ 371 Date: Mar. 5, 2008

(30) Foreign Application Priority Data
Oct. 27, 2005 (JP) PCTJP2005020154

(51) Int. Cl. A61K 38/43; A61K 38/10; A61P 43/00

(52) U.S. Cl. 424/94.1; 514/13

(57) ABSTRACT

The present invention relates to hepatic fibrosis-suppressing agents that are suitable for treating or preventing fibrotic liver diseases such as cirrhosis, which comprise as an active ingredient a substance that inhibits the production or accumulation of chondroitin sulfate proteoglycans including chondroitinase ABC and ADAMTS-4; and methods of screening for the agents.

The present inventors discovered for the first time that hepatic fibrosis could be efficiently suppressed by suppressing the production or accumulation of chondroitin sulfate proteoglycans. Specifically, fibrosis of liver tissues can be suppressed by administering chondroitinase ABC, a chondroitin sulfate proteoglycan-degrading enzyme, or by using siRNA to suppress the expression of C4ST-1, C6ST-1, or C6ST-2, a sulfotransferase for chondroitin sulfate proteoglycans. Compounds such as nucleic acids that are used as siRNA can be used as effective agents for suppressing hepatic fibrosis. Furthermore, hepatic fibrosis-suppressing agents can be found by screening for compounds that suppress the production or accumulation of chondroitin sulfate proteoglycans.

### TECHNICAL FIELD

The present invention relates to agents for suppressing hepatic fibrosis, methods for suppressing hepatic fibrosis, and methods for treating or preventing hepatic fibrotic diseases using the above methods.

### BACKGROUND ART

Chronic fibrotic liver disease is a general name for the terminal stage of chronic inflammatory conditions that can be developed in the liver, and is accompanied by excessive tissue fibrosis. Chronic fibrotic liver disease is a group of severe diseases which often lead to death through progressive liver dysfunction. There has been no radical therapeutic method for the disease. Currently, liver transplantation therapy is used against cirrhosis; however, such therapy significantly impairs patients' quality of life (QOL), and many cases show recurrence of fibrosis in the transplanted organ. Recently, TGF-β inhibitors, angiotensin inhibitors, and such have been expected as novel therapeutic agents for the fibrotic disease; however, none of these has an established efficacy. Thus, development of new agents that can improve patients' QOL, and effectively inhibit or retard the progress of hepatic fibrosis is in strong demand.

Currently, the following are expected as candidate therapeutic agents for fibrotic liver diseases: adrenal cortical steroids, colchicine, IL-10, and the like, which suppress chronic inflammation; TGF-β inhibitors, HGF (suppressing the activity of TGF-β), endothelin inhibitors, angiotensin inhibitors, and such, which activate fibroblasts; matrix metalloproteinase (MMP) which removes collagen, and others. However, there has been no established therapeutic method using any of these (Non-Patent Document 1 [the most recent review], and Non-Patent Documents 2 and 3).

Proteoglycan (PG) is a molecule with a structure of one or more glycosaminoglycan (GAG) chains covalently linked to a protein called the core protein. The specific sugar chain structure of GAG chains is believed to be responsible for various functions of PG. Based on the type of GAG chain, PGs are roughly divided into four groups: chondroitin sulfate proteoglycan (CSPG), dermatan sulfate proteoglycan (DSPG), heparan sulfate proteoglycan (HSPG), and keratan sulfate proteoglycan (KSPG) (Non-Patent Documents 4 and 5 [reviews]). Among others, HSPG has been extensively studied because its function is greatly modified through binding with various cytokines and chemokines.

Meanwhile, chondroitin sulfate proteoglycan (CSPG) is an essential molecule in the fetal period and is abundant in organs, but it is known to decrease along with birth, growth, and aging. Surprisingly, because of the diversity of its characteristics, the in vivo functions of CSPG remain to be clarified. Meanwhile, there are reports describing that CSPG is increased in diseased organs of cirrhosis, pulmonary fibrosis or such (Non-Patent Documents 6 and 7). However, the significance of CSPG increase in such diseases still remains unknown. In recent years, the finding that mice that are genetically deficient in CSPG show embryonic lethality or organogenesis failure has increased the recognition that CSPG is an essential molecule in the body.

Chondroitinase is known as a bacterial enzyme having an activity of degrading chondroitin sulfates (chondroitin sulfate proteoglycans). Known therapeutic agents that use chondroitinase include therapeutic agents for disk herniation that use chondroitinase ABC (Patent Document 1), wound healing-enhancing agents that use chondroitinase AC or chondroitinase B (Patent Document 2), and therapeutic agents for scleroderma, psoriasis, keloid or pulmonary fibrosis that use chondroitinase AC or chondroitinase B (Patent Document 3).

Patent Document 1: U.S. Pat. No. 6,001,630 specification
Patent Document 2: U.S. Pat. No. 5,997,863 specification
Patent Document 3: International Patent Application Publication WO 2001/039795 pamphlet
Non-Patent Document 1: Bataller R & Brenner D A, J. Clin. Invest. (2005) 115:209-218

### Non-Patent Document 2: Iredale J P, BMJ (2004) 327:143-147

Non-Patent Document 3: Bissell D M, Exp. Mol. Med. (2001) 33:179-190

### Non-Patent Document 4: Lin X, Development (2004) 131:6009-6021

Non-Patent Document 5: Hacker U et al., Nature Rev. Cell Biol. (2005) 6:530-541

### Non-Patent Document 6: Kovalski I et al., Orv Hetil. (1993) 134:2019-2026

### Non-Patent Document 7: Westergren-Thorsson G et al., J Clin Invest. (1993) 92:632-637

### DISCLOSURE OF THE INVENTION

An objective of the present invention is to provide agents that are capable of suppressing liver tissue fibrosis and useful in treating or preventing fibrotic liver diseases, therapeutic agents for fibrotic liver diseases comprising the agents as an active ingredient, and methods of screening for hepatic fibrosis-suppressing agents.

The present inventors conducted dedicated studies to develop such agents, and contemplated that excess accumulation of chondroitin sulfate proteoglycans (CSPGs) might enhance liver tissue fibrosis, although it was not considered as a cause of fibrotic liver diseases. The present inventors carried out research based on this assumption, and as a result discovered that chondroitinase ABC, a chondroitin sulfate proteoglycan-degrading enzyme, could efficiently degrade CSPGs accumulated in fibrotic livers and that fibrosis of liver tissues could be markedly suppressed by administering chondroitinase ABC in vivo. Furthermore, a similar therapeutic effect could be obtained by administering a recombinant peptide of a protein called ADAMTS-4 (A disintegrin and metalloproteinase with thrombospondin motifs) which has an activity of cleaving the core protein of versican (a chondroitin sulfate proteoglycan). Specifically, the present inventors demonstrated hepatic fibrosis could be suppressed by inhibiting the accumulation or biosynthesis of chondroitin sulfate proteoglycans. Accordingly, the inventors completed the present invention.

The present invention relates to hepatic fibrosis-suppressing agents, therapeutic agents for fibrotic liver diseases comprising the agents as an active ingredient, methods of screening for hepatic fibrosis-suppressing agents, and such. More specifically, the present invention provides:

[1] a hepatic fibrosis-suppressing agent comprising as an active ingredient a substance that inhibits the production or accumulation of a chondroitin sulfate proteoglycan;
[2] the agent of [1], wherein the substance has an activity of promoting the degradation of a chondroitin sulfate proteoglycan;
[3] the agent of [1], wherein the substance has an activity of inhibiting the synthesis of a chondroitin sulfate proteoglycan;
[4] the agent of [1], wherein the substance has an activity of desulfating a chondroitin sulfate proteoglycan;
[5] the agent of [1], wherein the substance has an activity of inhibiting the sulfation of a chondroitin sulfate proteoglycan;
[6] the agent of any one of [1] to [5], wherein the production or accumulation of a chondroitin sulfate proteoglycan is inhibited in liver;
[7] the agent of any one of [1] to [6], which is used for treating or preventing a fibrotic liver disease;
[8] the agent of [7], wherein the fibrotic liver disease is a chronic liver disease;
[9] the agent of [7], wherein the fibrotic liver disease is chronic hepatitis, liver fibrosis, cirrhosis, liver failure, or hepatocarcinoma;
[10] a method of screening for a hepatic fibrosis-suppressing agent, which comprises selecting from a test sample a substance with an activity of inhibiting the production or accumulation of a chondroitin sulfate proteoglycan;
[11] the screening method of [10], which comprises the step of selecting a substance with the activity of any one of
(a) promoting the degradation of a chondroitin sulfate proteoglycan,
(b) inhibiting the synthesis of a chondroitin sulfate proteoglycan,
(c) desulfating a chondroitin sulfate proteoglycan, and
(d) inhibiting the sulfation of a chondroitin sulfate proteoglycan; and
[12] the screening method of [10] or [11], wherein the hepatic fibrosis-suppressing agent is used for treating or preventing a fibrotic liver disease.

The present invention also includes the followings:

[13] a hepatic fibrosis-suppressing agent comprising chondroitinase ABC as an active ingredient;
[14] the hepatic fibrosis-suppressing agent of [13], which is used to treat or prevent a chronic liver disease;
[15] the hepatic fibrosis-suppressing agent of [14], in which the chronic liver disease is cirrhosis; and
[16] the hepatic fibrosis-suppressing agent of any one of [13] to [15], in which chondroitinase ABC is derived from Proteus vulgaris.

Further, the present invention relates to the followings:

[17] use of the agent of any one of [1] to [9] for producing a hepatic fibrosis-suppressing agent;
[18] a method of treating a hepatic fibrosis which comprises the step of administering the agent of any one of [1] to [9] to a subject (such as patients); and
[19] a composition which comprises the agent of any one of [1] to [9] and a pharmaceutically acceptable carrier.

The present invention has demonstrated that the production and accumulation of chondroitin sulfate proteoglycans is involved in the development of cirrhosis. Inhibiting the production or accumulation of chondroitin sulfate proteoglycans was shown to suppress the onset of hepatic fibrosis. Thus, therapeutic agents for fibrotic liver diseases can be provided based on this new concept. In particular, hepatic fibrosis is closely involved in chronic liver diseases such as chronic hepatitis, liver fibrosis, cirrhosis, liver failure, and hepatocarcinoma. Since the number of patients with these diseases is increasing in today's society, the new-concept therapeutic agents have great medical and industrial significances.

### BRIEF DESCRIPTION OF THE DRAWINGS

FIG. 1 presents photographs showing results of CSPG, C4SPG, C6SPG, and HSPG (brown signals) detected in carbon tetrachloride-induced cirrhosis model mice. The magnification was 100 fold. Names in the parentheses represent antibody clones used in the detection.

FIG. 2 presents photographs showing results of CSPG (brown signals) detected in carbon tetrachloride-induced cirrhotic liver after treatment with PBS (chondroitinase (−)), Chase AC, Chase B, or Chase ABC. The magnification was 200 fold.

FIG. 3 presents photographs showing the effect of Chase ABC administration on reducing the level of type III collagen (red signals) in carbon tetrachloride-induced cirrhosis model mice. The magnification was 100 fold.

FIG. 4 is a graph showing fibrosis scores in carbon tetrachloride-induced cirrhosis model mice after administration of Chase ABC, Chase AC, or Chase B. *p<0.05, **p<0.01 (t test).

FIG. 5 presents photographs showing the effect of Chase ABC administration on reducing the level of type I collagen (red signals) in carbon tetrachloride-induced cirrhosis model mice. The magnification was 100 fold.

FIG. 6 is a graph showing the type III collagen-positive area (%) in carbon tetrachloride-induced cirrhosis model mice after administration of Chase ABC, Chase AC, or Chase B. *p<0.05, **p<0.01 (t test).

FIG. 7 presents photographs showing results of CSPG, C4SPG; and C6SPG (brown signals) detected in carbon tetrachloride-induced cirrhosis model mice after Chase ABC administration. The magnification was 200 fold.

FIG. 8 presents photographs showing the distribution of fibroblasts (brown signals) and changes in the distribution after Chase ABC administration in carbon tetrachloride-induced cirrhosis model mice. In the upper panels, the magnification was 100 fold. In the bottom panels, the magnification was 200 fold.

FIG. 9 presents photographs showing the distribution of macrophages (brown signals) and changes in the distribution after Chase ABC administration in carbon tetrachloride-induced cirrhosis model mice. The magnification was 200 fold.

FIG. 10 presents photographs showing the suppression of fibroblast infiltration by administering a functional ADAMTS-4 peptide. Images of an ER-TR7-staining (brown) pattern in liver tissues of the cirrhosis model are shown (upper, 100 fold; bottom, 400 fold). The images demonstrate that administration of the functional ADAMTS-4 peptide not only suppressed cellular infiltration but also suppressed pseudolobule formation.

FIG. 11 presents photographs showing the suppression of type III collagen proliferation by administering a functional ADAMTS-4 peptide. Image of a staining pattern of type III collagen (brown) in liver tissues of the cirrhosis model is shown (100 fold). The image demonstrates that administration of the functional ADAMTS-4 peptide suppressed pseudolobule formation.

FIG. 12 is a graph showing the suppression of fibrosis by administering a functional ADAMTS-4 peptide. This graph was obtained by numerically evaluating the staining pattern of type III collagen as a fibrosis score. Administration of the functional ADAMTS-4 peptide was demonstrated to significantly suppress hepatic fibrosis.

### BEST MODE FOR CARR IN OUT THE INVENTION

The present invention is further specifically described below:

Pathological conditions associated with cirrhosis, a representative chronic liver disease, include fibrosis of liver. The present inventors focused on the functions of chondroitin sulfate proteoglycans to establish that the improvement of fibrotic conditions in liver is an effective therapeutic method for cirrhosis. The inventors then suppressed the accumulation of chondroitin sulfate proteoglycans in mouse models for cirrhosis, and closely analyzed this condition to reveal that the accumulation of chondroitin sulfate proteoglycans was ameliorated in many cells, and pathological condition of cirrhosis was also improved as compared with that in the liver of wild-type mice. Specifically, the inventors discovered that inhibiting the production or accumulation of chondroitin sulfate proteoglycans facilitated the amelioration of abnormal accumulations of chondroitin sulfate proteoglycans in a liver, a factor deeply involved in cirrhosis, and thus leading to the improvement of hepatic fibrosis.

The present invention relates to hepatic fibrosis-suppressing agents, which comprise substances that inhibit the production or accumulation of chondroitin sulfate proteoglycans as active ingredients.

In the present invention, “chondroitin sulfate proteoglycans” are a type of proteoglycan and collectively refer to compounds in which proteins (core proteins) are covalently linked to chondroitin sulfate/dermatan sulfate, which are representative sulfated mucopolysaccharides. In the present invention, preferable “chondroitin sulfate proteoglycans” are human chondroitin sulfate proteoglycans. The species from which the proteoglycans are derived are not particularly limited. Proteins of nonhuman organisms (homologues, orthologues, and such) that are equivalent to the chondroitin sulfate proteoglycans are also included in the “chondroitin sulfate proteoglycans” of the present invention. For example, the present invention can be conducted as long as the species has a protein corresponding to a human chondroitin sulfate proteoglycan and equivalent to a human chondroitin sulfate proteoglycan. Furthermore, the chondroitin sulfate proteoglycans in the present invention also include so-called part-time proteoglycans, which are temporarily linked with glycosaminoglycan (GAG) chains to become proteoglycans upon inflammation or the like.

Examples of the chondroitin sulfate proteoglycans described below are aggrecan, versican, neurocan, brevican, β-glycan, decorin, biglycan, fibromodulin, and PG-Lb. However, the chondroitin sulfate proteoglycans in the present invention are not limited to these examples, and may be any substances with a chondroitin sulfate proteoglycan activity. Herein, chondroitin sulfate proteoglycan activities include, for example, cell adhesion ability and cell growth promotion. Those skilled in the art can assay chondroitin sulfate proteoglycan activities by assaying cell division and growth of tumor cells (for example, Caco-2 and HT-29 cells) in the presence of a protein containing a partial amino acid sequence of a chondroitin sulfate proteoglycan, or a protein with high homology to such a partial amino acid (typically 70% homology or higher, preferably 80% or higher, more preferably 90% or higher, and most preferably 95% or higher). Proteins with the effect of promoting cell division and growth can be evaluated as proteins with a chondroitin sulfate proteoglycan activity (Int. J. Exp. Pathol. 2005 August, 86(4), 219-29; and Histochem. Cell Biol. 2005 August, 124(2), 139-49). High homology means 50% or higher homology, preferably 70% or higher homology, more preferably 80% or higher homology, and still more preferably 90% or higher homology (for example, 95% or higher homology, or 96%, 97%, 98%, 99% or higher homology). Such homology can be determined using the mBLAST algorithm (Altschul et al, Proc. Natl. Acad. Sci. USA 1990, 87, 2264-8; Karlin and Altschul, Proc. Natl. Acad. Sci. USA 1993, 90, 5873-7).

Herein, “hepatic fibrosis” refers to fibrotic conditions in liver tissues. Such conditions include but are not limited to, for example, accumulation of extracellular matrix such as collagen and activation of fibroblasts in liver tissues.

Herein, “inhibition of production or accumulation” of chondroitin sulfate proteoglycans includes, for example, “promotion of degradation”, “inhibition of synthesis”, “desulfation”, and “inhibition of sulfation” of chondroitin sulfate proteoglycans; however, “inhibition of production or accumulation” is not limited thereto, and it refers to a reduction or loss of the amount, function or activity of a chondroitin sulfate proteoglycan, as compared to a comparison subject. Herein, “substances that inhibit the production or accumulation” of chondroitin sulfate proteoglycans are not particularly limited. Such substances are preferably “substances with the effect of promoting the degradation of chondroitin sulfate proteoglycans”, “substances with the effect of inhibiting the synthesis of chondroitin sulfate proteoglycans”, “substances with the effect of desulfating chondroitin sulfate proteoglycans”, or “substances with the effect of inhibiting the sulfation of chondroitin sulfate proteoglycans”.

“Promotion of degradation” of chondroitin sulfate proteoglycans includes, for example, inhibition of the expression of core proteins of chondroitin sulfate proteoglycans, and a reduction in the abundance of the core proteins. Herein, “core proteins of chondroitin sulfate proteoglycans” include, for example, aggrecan, versican, neurocan, and brevican for matrix-type chondroitin sulfate proteoglycans; and β-glycan, decorin, biglycan, fibromodulin, and PG-Lb for membrane chondroitin sulfate proteoglycans. Those described above are all examples, but the core proteins are not limited to these and a wide variety of proteins may serve as chondroitin sulfate proteoglycan cores.

“Expression” includes “transcription” from genes, “translation” into polypeptides, and “suppression of degradation” of proteins. The “expression of core proteins of chondroitin sulfate proteoglycans” refers to transcription and translation of the genes encoding core proteins of chondroitin sulfate proteoglycans, or production of core proteins of chondroitin sulfate proteoglycans through transcription and translation. Furthermore, the “function of core proteins of chondroitin sulfate proteoglycans” includes, for example, their function in binding to chondroitin sulfate, and in binding with other cellular components. Those skilled in the art can appropriately evaluate (measure) the various above-mentioned functions using general methods. Specifically, the evaluation can be performed using the methods described in the Examples herein below, or using the same methods with appropriate modifications.

The “promotion of degradation” of chondroitin sulfate proteoglycans may also be an increase in the expression of enzymes that cleave or degrade chondroitin sulfate proteoglycans, or of enzymes involved in the cleavage or degradation of proteoglycans. Such enzymes include, for example, metalloproteinases (for example, ADAMTS-1, ADAMTS-4, and ADAMTS-5), chondroitinase, and calpain I, but are not limited thereto. The “promotion of degradation” may be a reduction in the abundance of chondroitin sulfate proteoglycans caused by administering all or some of the enzymes.

Alternatively, “promotion of degradation” may be achieved by administering a substance that promotes the suppression of expression of chondroitin sulfate proteoglycans. Such substances include, for example, n-butylate, diethylcarbamazepine, tunicamycin, non-steroidal estrogen, and cyclofenil diphenol, but are not limited thereto.

Preferred embodiments of the “substance with the activity of promoting degradation” include, for example, compounds (nucleic acids) selected from the group consisting of:

(a) antisense nucleic acids against transcripts of the genes encoding the core proteins of chondroitin sulfate proteoglycans, or portions thereof;
(b) nucleic acids with the ribozyme activity of specifically cleaving transcripts of genes encoding core proteins of chondroitin sulfate proteoglycans; and
(c) nucleic acids with the activity of using RNAi effect to inhibit the expression of genes encoding core proteins of chondroitin sulfate proteoglycans.

Furthermore, the “substances with the activity of promoting degradation” include, for example, compounds selected from the group consisting of:

(a) antibodies that bind to core proteins of chondroitin sulfate proteoglycans;
(b) chondroitin sulfate proteoglycan variants that are dominant-negative for core proteins of chondroitin sulfate proteoglycans; and
(c) low-molecular-weight compounds that bind to core proteins of chondroitin sulfate proteoglycans.

“Inhibition of synthesis” of chondroitin sulfate proteoglycans includes, for example, inhibition of biosynthesis of glycosaminoglycans and inhibition of enzymes involved in the synthesis of chondroitin sulfate proteoglycans, but is not limited thereto. The inhibition refers to inhibition of any of the processes of chondroitin sulfate proteoglycan synthesis.

As substances that inhibit the synthesis of chondroitin sulfate proteoglycans, substances inhibiting the biosynthesis of glycosaminoglycans include, for example, β-D-xyloside, 2-deoxy-D-glucose (2-DG), ethane-1-hydroxy-1,1-diphosphonate (ETDP), and 5-hexyl-2-deoxyuridine (HudR). Such substances inhibit the biosynthesis of glycosaminoglycans, and thereby inhibit the synthesis of chondroitin sulfate proteoglycans.

Meanwhile, enzymes involved in chondroitin synthesis include, for example, GalNAc4ST-1, GalNAc4ST-2, GALNAC4S-6ST, UA20ST, GalT-I, GaiT-II, GlcAT-I, and XylosylT. The synthesis of chondroitin sulfate proteoglycans is inhibited by inhibiting such enzymes, suppressing the expression thereof, or the like.

Preferred embodiments of “substances with the activity of inhibiting synthesis” include, for example, compounds (nucleic acids) selected from the group consisting of:

(a) antisense nucleic acids against transcripts of genes encoding chondroitin sulfate proteoglycan synthetases, or portions thereof,
(b) nucleic acids with the ribozyme activity of specifically cleaving transcripts of the genes encoding chondroitin sulfate proteoglycan synthetases; and
(c) nucleic acids with the activity of using RNAi effect to inhibit the expression of genes encoding chondroitin sulfate proteoglycan synthetases.

The “substances with the activity of inhibiting synthesis” also include, for example, compounds selected from the group consisting of:

(a) antibodies that bind to chondroitin sulfate proteoglycan synthetases;
(b) chondroitin sulfate proteoglycan synthetase variants that are dominant-negative for chondroitin sulfate proteoglycan synthetases; and
(c) low-molecular-weight compounds that bind to chondroitin sulfate proteoglycan synthetases.

The “desulfation” of chondroitin sulfate proteoglycans refers to the removal of a sulfate group from chondroitin sulfate proteoglycans, and includes, for example, desulfation by an endogenous or exogenously-administered desulfation enzyme, and suppression of sulfation by a sulfation-suppressing compound, but is not limited thereto. “Desulfation” refers to the process of sulfate group removal.

Such desulfation enzymes include, for example, chondroitin-4-sulfatase and chondroitin-6-sulfatase. Sulfation-suppressing compounds include, for example, chlorate and EGF receptor antagonists.

Preferred embodiments of such “substances with desulfating activity” include, for example, compounds (nucleic acids) selected from the group consisting of:

(a) antisense nucleic acids against transcripts of genes encoding proteins that suppress chondroitin sulfate proteoglycan-desulfating enzymes, or portions thereof,
(b) nucleic acids with the ribozyme activity of specifically cleaving transcripts of the genes encoding proteins that suppress chondroitin sulfate proteoglycan-desulfating enzymes; and
(c) nucleic acids with the activity of using RNAi effect to inhibit the expression of genes encoding proteins that suppress chondroitin sulfate proteoglycan-desulfating enzymes.

The “substances with desulfating activity” also include, for example, compounds selected from the group consisting of:

(a) antibodies that bind to compounds that suppress chondroitin sulfate proteoglycan-desulfating enzymes;
(b) variants of proteins that suppress chondroitin sulfate proteoglycan-desulfating enzymes, which are dominant-negative for proteins that suppress chondroitin sulfate proteoglycan-desulfating enzymes; and
(c) low-molecular-weight compounds that bind to compounds that suppress chondroitin sulfate proteoglycan-desulfating enzymes.

Herein, “desulfation-suppressing compounds” include not only proteins but also non-proteinacious compounds such as coenzymes.

The “activity of inhibiting sulfation” of chondroitin sulfate proteoglycans includes, for example, inhibition of sulfotransferases, but is not limited thereto. The activity refers to the inhibition of sulfation in the process of chondroitin sulfate proteoglycan synthesis.

Such sulfotransferases include, for example, chondroitin D-N-acetylgalactosamine-4-O-sulfotransferase 1 (C4ST-1), chondroitin D-N-acetylgalactosamine-4-O-sulfotransferase 2 (C4ST-2), chondroitin D-N-acetylgalactosamine-4-O-sulfotransferase 3 (C4ST-3), D4ST, C6ST-1, and C6ST-2.

Preferred embodiments of “substances with the activity of inhibiting sulfation” include, for example, compounds (nucleic acids) selected from the group consisting of:

(a) antisense nucleic acids against transcripts of genes encoding sulfotransferases for chondroitin sulfate proteoglycans, or portions thereof;
(b) nucleic acids with the ribozyme activity of specifically cleaving transcripts of the genes encoding sulfotransferases for chondroitin sulfate proteoglycans; and
(c) nucleic acids with the activity of using RNAi effect to inhibit the expression of genes encoding sulfotransferases for chondroitin sulfate proteoglycans.

The “substances with the activity of inhibiting sulfation” also include, for example, compounds selected from the group consisting of:

(a) antibodies that bind to sulfotransferases for chondroitin sulfate proteoglycans;
(b) sulfotransferase variants for chondroitin sulfate proteoglycans; and
(c) low-molecular-weight compounds that bind to sulfotransferases for chondroitin sulfate proteoglycans.

The above enzymes shown as examples include not only single enzymes that correspond to single genes, but also groups of enzymes that share certain characteristics. For example, chondroitinase is a collective name for enzymes such as ABC, AC, and B, whose substrate specificities or such are different, but which share the characteristics of mucopolysaccharide-degrading enzymes. For example, chondroitinase AC I eliminatively cleaves the N-acetylhexosaminide linkages of chondroitin sulfates (A, C, and E), chondroitin, chondroitin sulfate-dermatan sulfate hybrids, and hyaluronic acid, and yields oligosaccharides with Δ4-glucuronate residues at the non-reducing ends. This enzyme does not act on dermatan sulfate (chondroitin sulfate B, which has L-iduronic acid for a hexuronic acid), keratan sulfate, heparan sulfate, and heparin. Meanwhile, chondroitinase AC II eliminatively cleaves the N-acetylhexosaminide linkages of chondroitin, chondroitin sulfate A, and chondroitin sulfate C, and yields Δ4-unsaturated disaccharides (ΔDi-0S, ΔDi-4S, and ΔDi-6S). This enzyme also acts well on hyaluronic acid. The enzyme does not act on dermatan sulfate (chondroitin sulfate B), which is therefore a competitive inhibitor of the enzyme. Chondroitinase B (dermatanase) eliminatively cleaves N-acetylhexosaminide linkages to L-iduronic acids in dermatan sulfate, and yields oligosaccharides (di- and tetra-saccharides) with Δ4-hexuronate residues at the non-reducing ends. This enzyme acts on neither chondroitin sulfate A nor chondroitin sulfate C, which are free of L-iduronic acid. Dermatan, which is a derivative of dermatan sulfate in which the sulfate group is removed, does not serve as a substrate for this enzyme. This enzyme preferentially cleaves portions of dermatan sulfate in which the second of the L-iduronic acid units are sulfated. Chondroitinase ABC eliminatively cleaves N-acetylhexosaminide linkages of chondroitin sulfate A, chondroitin sulfate C, dermatan sulfate, chondroitin, and hyaluronic acid, and yields mainly disaccharides with Δ4-hexuronate groups at the non-reducing ends. This enzyme does not act on keratan sulfate, heparin, and heparan sulfate. Chondroitinases collectively refer to enzymes sharing the characteristics of mucopolysaccharide-degrading enzymes while also having different characteristics as described above, and they are not limited to chondroitinase AC I, chondroitinase AC II, chondrotinase B, and chondroitinase ABC as exemplified above.

Further, on a genomic DNA level, such groups of enzymes sharing features do not necessarily correspond to single genes. For example, both chondroitin-4-sulfatase and chondroitin-6-sulfatase can be retrieved from the public gene database Genbank as sequences referred to by multiple accession numbers (for example, Genbank Accession Nos: NT_{—}039500 (a portion thereof is shown under Accession No: CAAA01098429 (SEQ ID NO: 68)), NT_{—}078575, NT_{—}039353, NW_{—}001030904, NW_{—}001030811, NW_{—}001030796, and NW_{—}000349).

The above example proteins that correspond to single genes are shown below: Specifically, below are the accession numbers in the public gene database Genbank, nucleotide sequences, and amino acid sequences for human genes encoding:

aggrecan, versican, neurocan, brevican, β-glycan, decorin, biglycan, fibromodulin, and PG-Lb, which are shown above as examples of chondroitin sulfate proteoglycans; ADAMTS-1, ADAMTS-4, ADAMTS-5, and calpain I, which are shown above as examples of enzymes that cleave or degrade chondroitin sulfate proteoglycans or related enzymes; GalNAc4ST-1, GalNAc4ST-2, GALNAC4S-6ST, UA2OST, GalT-I, GalT-II, GlcAT-I, and XylosylT, which are shown above as examples of enzymes involved in chondroitin synthesis; C4ST-1, C4ST-2, C4ST-3, D4ST, C6ST-1, and C6ST-2, which are shown above as examples of sulfotransferases.
Aggrecan (Accession No: NM_{—}007424; nucleotide sequence: SEQ ID NO: 1; amino acid sequence: SEQ ID NO: 2)
Versican (Accession No: BC096495; nucleotide sequence: SEQ ID NO: 3; amino acid sequence: SEQ ID NO: 4)
Neurocan (Accession No: NM_{—}010875; nucleotide sequence: SEQ ID NO: 5; amino acid sequence: SEQ ID NO: 6)
Brevican (Accession No: NM_{—}007529; nucleotide sequence: SEQ ID NO: 7; amino acid sequence: SEQ ID NO: 8)
β-glycan (Accession No: AF039601; nucleotide sequence: SEQ ID NO: 9; amino acid sequence: SEQ ID NO: 10)
Decorin (Accession No: NM_{—}007833; nucleotide sequence: SEQ ID NO: 11; amino acid sequence: SEQ ID NO: 12)
Biglycan (Accession No: BC057185; nucleotide sequence: SEQ ID NO: 13; amino acid sequence: SEQ ID NO: 14)
Fibromodulin (Accession No: NM_{—}021355; nucleotide sequence: SEQ ID NO: 15; amino acid sequence: SEQ ID NO: 16)
PG-Lb (Accession No: NM_{—}007884; nucleotide sequence: SEQ ID NO: 17; amino acid sequence: SEQ ID NO: 18)
ADAMTS-1 (Accession No: NM_{—}009621; nucleotide sequence: SEQ ID NO: 19; amino acid sequence: SEQ ID NO: 20)
ADAMTS-4 (Accession No: NM_{—}172845; nucleotide sequence: SEQ ID NO: 21; amino acid sequence: SEQ ID NO: 22)
ADAMTS-5 (Accession No: AF140673; nucleotide sequence: SEQ ID NO: 23; amino acid sequence: SEQ ID NO: 24)
Calpain I (Accession No: NM_{—}007600; nucleotide sequence: SEQ ID NO: 25; amino acid sequence: SEQ ID NO: 26)
GalNAc4ST-1 (Accession No: NM_{—}175140; nucleotide sequence: SEQ ID NO: 27; amino acid sequence: SEQ ID NO: 28)
GalNAc4ST-2 (Accession No: NM_{—}199055; nucleotide sequence: SEQ ID NO: 29; amino acid sequence: SEQ ID NO: 30)
GALNAC4S-6ST (Accession No: NM_{—}029935; nucleotide sequence: SEQ ID NO: 31; amino acid sequence: SEQ ID NO: 32)
UA2OST (Accession No: NM_{—}177387; nucleotide sequence: SEQ ID NO: 33; amino acid sequence: SEQ ID NO: 34)
GalT-I (Accession No: NM_{—}016769; nucleotide sequence: SEQ ID NO: 35; amino acid sequence: SEQ ID NO: 36)
GalT-II (Accession No: BC064767; nucleotide sequence: SEQ ID NO: 37; amino acid sequence: SEQ ID NO: 38)
GlcAT-I (Accession No: BC058082; nucleotide sequence: SEQ ID NO: 39; amino acid sequence: SEQ ID NO: 40), or Accession No: NM_{—}024256; nucleotide sequence: SEQ ID NO: 41; amino acid sequence: SEQ ID NO: 42)
XylosylT (Accession No: NM_{—}145828; nucleotide sequence: SEQ ID NO: 43; amino acid sequence: SEQ ID NO: 44)
C4ST-1 (Accession No: NM_{—}021439; nucleotide sequence: SEQ ID NO: 45; amino acid sequence: SEQ ID NO: 46)
C4ST-2 (Accession No: NM_{—}021528; nucleotide sequence: SEQ ID NO: 47; amino acid sequence: SEQ ID NO: 48)
C4ST-3 (Accession No: XM_{—}355798; nucleotide sequence: SEQ ID NO: 49; amino acid sequence: SEQ ID NO: 50)
D4ST (Accession No: NM_{—}028117; nucleotide sequence: SEQ ID NO: 51; amino acid sequence: SEQ ID NO: 52)
C6ST-1 (Accession No: NM_{—}016803; nucleotide sequence: SEQ ID NO: 53; amino acid sequence: SEQ ID NO: 54)
C6ST-2 (Accession No: AB046929; nucleotide sequence: SEQ ID NO: 55; amino acid sequence: SEQ ID NO: 56)

In addition to the proteins listed above, the proteins of the present invention include those exhibiting high homology (typically 70% or higher, preferably 80% or higher, more preferably 90% or higher, and most preferably 95% or higher) to sequences shown in the Sequence Listing and with a function of the proteins listed above (for example, the function of binding to intracellular components). The proteins listed above are, for example, proteins comprising an amino acid sequence with an addition, deletion, substitution, or insertion of one or more amino acids in any of the amino acid sequences of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, and 56, in which the number of altered amino acids is typically 30 amino acids or less, preferably ten amino acids or less, more preferably five amino acids or less, and most preferably three amino acids or less.

The above-described genes of the present invention include, for example, endogenous genes of other organisms which correspond to DNAs comprising any of the nucleotide sequences of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, and 55 (homologues to the human genes described above, or the like).

Each of the endogenous DNAs of other organisms which correspond to DNAs comprising any of the nucleotide sequences of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, and 55 are generally highly homologous to a DNA of any of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, and 55. High homology means 50% or higher homology, preferably 70% or higher homology, more preferably 80% or higher homology, and still more preferably 90% or higher homology (for example, 95% or higher, or 96%, 97%, 98%, or 99% or higher). Homology can be determined using the mBLAST algorithm (Altschul et al., Proc. Natl. Acad. Sci. USA 1990, 87, 2264-8; Karlin and Altschul, Proc. Natl. Acad. Sci. USA 1993, 90, 5873-7). When the DNAs have been isolated from the body, each of them may hybridize under stringent conditions to a DNA of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, or 55. Herein, stringent conditions include, for example, “2×SSC, 0.1% SDS, 50° C.”, “2×SSC, 0.1% SDS, 42° C.”, and “1×SSC, 0.1% SDS, 37° C.”; more stringent conditions include “2×SSC, 0.1% SDS, 65° C.”, “0.5×SSC, 0.1% SDS, 42° C.”, and “0.2×SSC, 0.1% SDS, 65° C.”.

Those skilled in the art can appropriately obtain proteins functionally equivalent to the above-described proteins from the above-described highly homologous proteins by using methods for assaying the activity of promoting the degradation of CSPGs, inhibiting the synthesis of CSPGs, desulfating CSPGs, or inhibiting the sulfation of CSPGs. Specific methods for assaying the activities are described below in a section on the screening methods of the present invention. Further, based on the nucleotide sequences of the above-described genes, those skilled in the art can appropriately obtain endogenous genes of other organisms that correspond to the above-described genes. In the present invention, the above-described proteins and genes in non-human organisms, which correspond to the above-described proteins and genes, or the above-described proteins and genes that are functionally equivalent to the above-described proteins and genes, may simply be referred to using the above-described names.

The proteins of the present invention can be prepared not only as natural proteins but also as recombinant proteins using genetic recombination techniques. The natural proteins can be prepared by, for example, methods of subjecting cell extracts (tissue extracts) that may express the above-described proteins to affinity chromatography using antibodies against the above-described proteins. On the other hand, the recombinant proteins can be prepared, for example, by culturing cells transformed with DNAs encoding the proteins described above. The above-described proteins of the present invention can be suitably used, for example, in the screening methods described herein below.

In the present invention, “nucleic acids” refer to both RNAs and DNAs. Chemically synthesized nucleic acid analogs, such as so-called “PNAs” (peptide nucleic acids), are also included in the nucleic acids of the present invention. PNAs are nucleic acids in which the fundamental backbone structure of nucleic acids, the pentose-phosphate backbone, is replaced by a polyamide backbone with glycine units. PNAs have a three-dimensional structure quite similar to that of nucleic acids.

Methods for inhibiting the expression of specific endogenous genes using antisense technology are well known to those skilled in the art. There are a number of causes for the action of antisense nucleic acids in inhibiting target gene expression, including: inhibition of transcription initiation by triplex formation;

transcription inhibition by hybrid formation at a site with a local open loop structure generated by an RNA polymerase;
transcription inhibition by hybrid formation with the RNA being synthesized;
splicing inhibition by hybrid formation at an intron-exon junction;
splicing inhibition by hybrid formation at the site of spliceosome formation;
inhibition of transport from the nucleus to the cytoplasm by hybrid formation with mRNA;
splicing inhibition by hybrid formation at the capping site or poly(A) addition site;
inhibition of translation initiation by hybrid formation at the translation initiation factor binding site;
inhibition of translation by hybrid formation at the ribosome binding site adjacent to the start codon;
inhibition of peptide chain elongation by hybrid formation in the translational region of mRNA or at the polysome binding site of mRNA; and
inhibition of gene expression by hybrid formation at the protein-nucleic acid interaction sites. Thus, antisense nucleic acids inhibit the expression of target genes by inhibiting various processes, such as transcription, splicing, and translation (Hirashima and Inoue, Shin Seikagaku Jikken Koza 2 (New Courses in Experimental Biochemistry 2), Kakusan (Nucleic Acids) IV: “Idenshi no Fukusei to Hatsugen (Gene replication and expression)”, Ed. The Japanese Biochemical Society, Tokyo Kagakudojin, 1993, pp. 319-347).

The antisense nucleic acids used in the present invention may inhibit the expression and/or function of genes encoding any of the CSPG core proteins, synthetases, proteins suppressing desulfation enzymes, and sulfotransferases described above, based on any of the actions described above. In one embodiment, antisense sequences designed to be complementary to an untranslated region adjacent to the 5′ end of an mRNA for a gene encoding an above-described CSPG core protein, synthetase, desulfation enzyme-suppressing protein, or sulfotransferase may be effective for inhibiting translation of the gene. Sequences complementary to a coding region or 3′-untranslated region can also be used. Thus, the antisense nucleic acids to be used in the present invention include not only nucleic acids comprising sequences antisense to the coding regions, but also nucleic acids comprising sequences antisense to untranslated regions of genes encoding the above-described CSPG core proteins, synthetases, desulfation enzyme-suppressing proteins, or sulfotransferases. Such antisense nucleic acids to be used are linked downstream of adequate promoters and are preferably linked with transcription termination signals on the 3′ side. Nucleic acids thus prepared can be introduced into desired animals (cells) using known methods. The sequences of the antisense nucleic acids are preferably complementary to a gene or portion thereof encoding a CSPG core protein, synthetase, desulfation enzyme-suppressing protein, or sulfotransferase that is endogenous to the animals (cells) to be transformed with them. However, the sequences need not be perfectly complementary, as long as the antisense nucleic acids can effectively suppress expression of a gene. The transcribed RNAs preferably have 90% or higher, and most preferably 95% or higher complementarity to target gene transcripts. To effectively inhibit target gene expression using antisense nucleic acids, the antisense nucleic acids are preferably at least 15 nucleotides long, and less than 25 nucleotides long. However, the lengths of the antisense nucleic acids of the present invention are not limited to the lengths mentioned above, and they may be 100 nucleotides or more, or 500 nucleotides or more.

The antisense nucleic acids of the preset invention are not particularly limited, and can be prepared, for example, based on the nucleotide sequence of a versican gene (GenBank Accession No: BC096495; SEQ ID NO: 3), C4ST-1 (GenBank Accession No: NM_{—}021439; SEQ ID NO: 45), C4ST-2 (GenBank Accession No: NM_{—}021528; SEQ ID NO: 47), C4ST-3 (GenBank Accession No: XM_{—}355798; SEQ ID NO: 49), or such.

Expression of the above-mentioned genes encoding CSPG core proteins, synthetases, desulfation enzyme-suppressing proteins, or sulfotransferases can also be inhibited using ribozymes or ribozyme-encoding DNAs. Ribozymes refer to RNA molecules with catalytic activity. There are various ribozymes with different activities. Among others, studies that focused on ribozymes functioning as RNA-cleaving enzymes have enabled the design of ribozymes that cleave RNAs in a site-specific manner. Some ribozymes have 400 or more nucleotides, such as group I intron type ribozymes and M1 RNA, which is comprised by RNase P, but others, called hammerhead and hairpin ribozymes, have a catalytic domain of about 40 nucleotides (Koizumi, M. and Otsuka, E., Tanpakushitsu Kakusan Koso (Protein, Nucleic Acid, and Enzyme) 1990, 35, 2191).

For example, the autocatalytic domain of a hammerhead ribozyme cleaves the sequence G13U14C15 at the 3′ side of C15. Base pairing between U14 and A9 has been shown to be essential for this activity, and the sequence can be cleaved when C15 is substituted with A15 or U15 (Koizumi, M. et al., FEBS Lett. 1988, 228, 228). Restriction enzyme-like RNA-cleaving ribozymes that recognize the sequence UC, WU, or UA in target RNAs can be created by designing their substrate-binding sites to be complementary to an RNA sequence adjacent to a target site (Koizumi, M. et al., FEBS Lett. 1988, 239, 285; Koizumi, M. and Otsuka, E., Tanpakushitsu Kakusan Koso (Protein, Nucleic Acid, and Enzyme) 1990, 35, 2191; and Koizumi, M. et al., Nucl Acids Res. 1989, 17, 7059).

In addition, hairpin ribozymes are also useful for the purposes of the present invention. Such ribozymes are found in, for example, the minus strand of satellite RNAs of tobacco ringspot viruses (Buzayan, J. M., Nature 1986, 323, 349). It has been shown that target-specific RNA-cleaving ribozymes can also be created from hairpin ribozymes (Kikuchi, Y. and Sasaki, N., Nucl Acids Res. 1991, 19, 6751; and Kikuchi, Y. Kagaku to Seibutsu (Chemistry and Biology) 1992, 30, 112). Thus, the expression of the above-described genes encoding CSPG core proteins, synthetases, desulfation enzyme-suppressing proteins, or sulfotransferases can be inhibited by using ribozymes to specifically cleave the gene transcripts.

The expression of endogenous genes can also be suppressed by RNA interference (hereinafter abbreviated as “RNAi”), using double-stranded RNAs comprising a sequence the same as or similar to a target gene sequence.

A great many disease-related genes have been rapidly identified since the entire human nucleotide sequence was revealed upon the recent completion of the genome project, and currently specific gene-targeted therapies and drugs are being actively developed. Of these, the application to gene therapy of small interfering RNAs (siRNAs), which produce the effect of specific post-transcriptional suppression, has been drawing attention. RNAi is a technology currently drawing attention in which double-stranded RNAs (dsRNAs) incorporated directly into cells suppress the expression of genes with sequences homologous to the dsRNAs. In mammalian cells, RNAi can be induced using short dsRNAs (siRNAs) and has many advantages: compared to knockout mice, RNAi has a stable effect, simple experiments, low costs, and so on.

Nucleic acids with inhibitory activity based on RNAi effect are generally referred to as siRNAs or shRNAs. RNAi is a phenomenon in which, when cells or such are introduced with short double-stranded RNAs (hereinafter abbreviated as “dsRNAs”) comprising sense RNAs that comprise sequences homologous to the mRNAs of a target gene, and antisense RNAs that comprise sequences homologous a sequence complementary thereto, the dsRNAs bind specifically and selectively to the target gene mRNAs, induce their disruption, and cleave the target gene, thereby effectively inhibiting (suppressing) target gene expression. For example, when dsRNAs are introduced into cells, the expression of genes with sequences homologous to the RNAs is suppressed (the genes are knocked down). As described above, RNAi can suppress the expression of target genes, and is thus drawing attention as a method applicable to gene therapy, or as a simple gene knockout method replacing conventional methods of gene disruption, which are based on complicated and inefficient homologous recombination. The RNAs to be used in RNAi are not necessarily perfectly identical to the genes or portions thereof that encode an above-described CSPG core protein, synthetase, desulfation enzyme-suppressing protein, or sulfotransferase; however, the RNAs are preferably perfectly homologous to the genes or portions thereof.

The targets of the siRNAs to be designed are not particularly limited, as long as they are genes encoding an above-described CSPG core protein, synthetase, desulfation enzyme-suppressing protein, or sulfotransferase. Any region of the gene can be a candidate for a target. For example, siRNAs may be prepared based on a nucleotide sequence of the versican gene (SEQ ID NO: 3), C4ST-1 gene (SEQ ID NO: 45), C4ST-2 gene (SEQ ID NO: 47), C4ST-3 gene (SEQ ID NO: 49), and such. More specifically, partial regions of such sequences may be used as candidates for the targets. For example, siRNAs may be prepared based on portions of the nucleotide sequences of a versican gene (SEQ ID NO: 57), C4ST-1 gene (SEQ ID NO: 58), C4ST-2 gene (SEQ ID NO: 59), C4ST-3 gene (SEQ ID NO: 60), C6ST-1 gene (SEQ ID NO: 61), C6ST-2 gene (SEQ ID NO: 62), GalNAc4ST-1 gene (SEQ ID NO: 63), GalNAc4ST-2 gene (SEQ ID NO: 64), GALNAC4S-6ST gene (SEQ ID NO: 65), or such.

The siRNAs can be introduced into cells by adopting methods of introducing cells with plasmid DNAs linked with siRNAs synthesized in vitro or methods that comprise annealing two RNA strands.

The two RNA molecules described above may be closed at one end or, for example, may be siRNAs with hairpin structures (shRNAs). shRNAs refer to short hairpin RNAs, which are RNA molecules with a stem-loop structure, since a portion of the single strand constitutes a strand complementary to another portion. Thus, molecules capable of forming an intramolecular RNA duplex structure are also included in the siRNAs of the present invention.

In a preferred embodiment of the present invention, the siRNAs of the present invention also include, for example, double-stranded RNAs with additions or deletions of one or a few RNAs in an siRNA which targets a specific DNA sequence (SEQ ID NOs: 57 to 65) shown herein and which can suppress the expression of versican, C4ST-1, C4ST-2, C4ST-3, or such via RNAi effect, as long as the double-stranded RNAs have the function of suppressing the expression of a gene encoding an above-described CSPG core protein, synthetase, desulfation enzyme-suppressing protein, or sulfotransferase.

The RNAs used in RNAi (siRNAs) do not need to be perfectly identical (homologous) to the genes encoding the above proteins or portions thereof; however, the RNAs are preferably perfectly identical (homologous).

Some details of the RNAi mechanism still remain unclear, but it is understood that an enzyme called “DICER” (a member of the RNase III nuclease family) is contacted with a double-stranded RNA and degrades it in to small fragments, called “small interfering RNAs” or “siRNAs”. The double-stranded RNAs of the present invention that have RNAi effect include such double-stranded RNAs prior to being degraded by DICER. Specifically, since even long RNAs that have no RNAi effect when intact can be degraded into siRNAs which have RNAi effect in cells, the length of the double-stranded RNAs of the present invention is not particularly limited.

For example, long double-stranded RNAs covering the full-length or near full-length mRNA of a gene encoding an above-described CSPG core protein, synthetase, desulfation enzyme-suppressing protein, or sulfotransferase can be pre-digested, for example, by DICER, and then the degradation products can be used as agents of the present invention. These degradation products are expected to contain double-stranded RNA (siRNA) molecules with RNAi effect. With this method, it is not necessary to specifically select the mRNA regions expected to have RNAi effect. In other words, it is not necessary to accurately determine regions with RNAi effect in the mRNAs of the genes described above.

The above-described “double-stranded RNAs capable of suppression via RNAi effect” can be suitably prepared by those skilled in the art based on nucleotide sequences of the above-described CSPG genes encoding core proteins, synthetases, desulfation enzyme-suppressing proteins, or sulfotransferases, which are targeted by the double-stranded RNAs. For example, the double-stranded RNAs of the present invention can be prepared based on the nucleotide sequence of SEQ ID NO: 57. In other words, it is within the range of ordinary trials for those skilled in the art to select an arbitrary consecutive RNA region in an mRNA that is a transcript of the nucleotide sequence of SEQ ID NO: 57, and prepare double-stranded RNA corresponding to the region. Those skilled in the art can also use known methods to properly select siRNA sequences with stronger RNAi effect from the mRNA sequence, which is the transcript of the nucleotide sequence of SEQ ID NO: 57. When one of the strands is already identified, those skilled in the art can readily determine the nucleotide sequence of the other strand (complementary strand). Those skilled in the art can appropriately prepare siRNAs using a commercially available nucleic acid synthesizer. Alternatively, general custom synthesis services may be used to synthesize desired RNAs.

The siRNAs of the present invention are not necessarily single pairs of double-stranded RNAs directed to target sequences, but may be mixtures of multiple double-stranded RNAs directed to regions that cover the target sequence. Herein, those skilled in the art can appropriately prepare the siRNAs as nucleic acid mixtures matched to a target sequence by using a commercially available nucleic acid synthesizer or DICER enzyme. Meanwhile, general custom synthesis services may be used to synthesize desired RNAs. The siRNAs of the present invention include so-called “siRNA cocktails”.

All nucleotides in the siRNAs of the present invention do not necessarily need to be ribonucleotides (RNAs). Specifically, one or more of the ribonucleotides constituting the siRNAs of the present invention may be replaced with corresponding deoxyribonucleotides. The term “corresponding” means that although the sugar moieties are structurally differently, the nucleotide residues (adenine, guanine, cytosine, or thymine (uracil)) are the same. For example, deoxyribonucleotides corresponding to ribonucleotides with adenine refer to deoxyribonucleotides with adenine. The term “or more” described above is not particularly limited, but preferably refers to a small number of about two to five ribonucleotides.

Furthermore, DNAs (vectors) capable of expressing the RNAs of the present invention are also included in the preferred embodiments of compounds capable of suppressing the expression of the genes encoding the above-described proteins of the present invention. The DNAs (vectors) capable of expressing the double-stranded RNAs of the present invention are, for example, DNAs structured such that a DNA encoding one strand of a double-stranded RNA and a DNA encoding the other strand of the double-stranded RNA are linked with promoters so that each DNA can be expressed. The above DNAs of the present invention can be appropriately prepared by those skilled in the art using standard genetic engineering techniques. More specifically, the expression vectors of the present invention can be prepared by adequately inserting DNAs encoding the RNAs of the present invention into various known expression vectors.

Furthermore, the expression-inhibiting substances of the present invention also include compounds that inhibit the expression of the above-described CSPG core proteins, synthetases, desulfation enzyme-suppressing proteins, or sulfotransferases by binding to an expression regulatory region of a gene encoding the above-described CSPG core proteins, synthetases, desulfation enzyme-suppressing proteins, or sulfotransferases (for example, a promoter region; specific examples include the nucleotide sequence of SEQ ID NO: 66, which is a promoter region of PG-Lb). Such compounds can be obtained, for example, using a fragment of a promoter DNA of the gene encoding an above-described CSPG core protein, synthetase, desulfation enzyme-suppressing protein, or sulfotransferase to perform screening methods using as an indicator the activity of binding to the DNA fragment. Those skilled in the art can appropriately determine whether compounds of interest inhibit the expression of the above-described genes encoding CSPG core proteins, synthetases, desulfation enzyme-suppressing proteins, or sulfotransferases by using known methods, for example, reporter assays and such.

Furthermore, DNAs (vectors) capable of expressing the above-described RNAs of the present invention are also included in preferred embodiments of the compounds capable of inhibiting the expression of a gene encoding an above-described CSPG core protein, synthetase, desulfation enzyme-suppressing protein, or sulfotransferase of the present invention. For example, DNAs (vectors) capable of expressing the above-described double-stranded RNAs of the present invention are structured such that a DNA encoding one strand of a double-stranded RNA and a DNA encoding the other strand of the double-stranded RNA are linked to promoters so that both can be expressed. Those skilled in the art can appropriately prepare the above-described DNAs of the present invention using standard genetic engineering techniques. More specifically, the expression vectors of the present invention can be prepared by appropriately inserting DNAs encoding the RNAs of the present invention into various known expression vectors.

Preferred embodiments of the above-described vector of the present invention include vectors expressing RNAs (siRNAs) that can suppress the expression of versican, C4ST-1, C4ST-2, C4ST-3, or the like by RNAi effect.

Antibodies that bind to the above-described CSPG core proteins, synthetases, desulfation enzyme-suppressing compounds, or sulfotransferases can be prepared by methods known to those skilled in the art. Polyclonal antibodies can be obtained, for example, by the following procedure: small animals such as rabbits are immunized with an above-described natural protein or a recombinant protein expressed in microorganisms as a fusion protein with GST, or a partial peptide thereof. Sera are obtained from these animals and purified by, for example, ammonium sulfate precipitation, Protein A or G column, DEAE ion exchange chromatography, affinity column coupled with the core protein, synthetase, desulfation enzyme-suppressing compound, or sulfotransferase for CSPGs described above, synthetic peptide, or such, to prepare antibodies. Monoclonal antibodies can be obtained by the following procedure: small animals such as mice are immunized with an above-described CSPG core protein, synthetase, desulfation enzyme-suppressing compound, or sulfotransferase, or a partial peptide thereof. Spleens are removed from the mice and crushed to isolate cells. The cells are fused with mouse myeloma cells using a reagent such as polyethylene glycol. Clones producing antibodies that bind to an above-described CSPG core protein, synthetase, desulfation enzyme-suppressing compound, or sulfotransferase are selected from among the resulting fused cells (hybridomas). The obtained hybridomas are then transplanted in the peritoneal cavities of mice, and ascites is collected from the mice. The obtained monoclonal antibodies can be purified by, for example, ammonium sulfate precipitation, Protein A or G columns, DEAE ion exchange chromatography, affinity columns coupled with an above-described CSPG core protein, synthetase, desulfation enzyme-suppressing compound, or sulfotransferase, synthetic peptides, or such.

The antibodies of the present invention are not particularly limited as long as they bind to an above-described core protein, synthetase, desulfation enzyme-suppressing compound, or sulfotransferase of the present invention. The antibodies of the present invention may be human antibodies, humanized antibodies created by gene recombination, fragments or modified products of such antibodies, in addition to the polyclonal and monoclonal antibodies described above.

The proteins of the present invention used as sensitizing antigens to prepare antibodies are not limited in terms of the animal species from which the proteins are derived. However, the proteins are preferably derived from mammals, for example, mice and humans. Human-derived proteins are particularly preferred. The human-derived proteins can be appropriately obtained by those skilled in the art using the gene or amino acid sequences disclosed herein.

In the present invention, the proteins to be used as sensitizing antigens may be whole proteins or partial peptides thereof. Such partial peptides of the proteins include, for example, amino-terminal (N) fragments and carboxyl-terminal (C) fragments of the proteins. Herein, “antibodies” refer to antibodies that react with a full-length protein or fragment thereof.

In addition to immunizing nonhuman animals with antigens to obtain the above hybridomas, human lymphocytes, for example, EB virus-infected human lymphocytes, can be sensitized in vitro with the proteins or with cells expressing the proteins, or with lysates thereof, and the sensitized lymphocytes can be fused with human-derived myeloma cells with the ability to divide permanently, for example, U266, to obtain hybridomas that produce desired human antibodies with binding activity to the proteins.

It is expected that antibodies against the above-described CSPG core proteins, synthetases, desulfation enzyme-suppressing compounds, or sulfotransferases of the present invention exhibit the effect of inhibiting protein expression or function by binding to the proteins. When using the prepared antibodies for human administration (antibody therapy), the antibodies are preferably human or humanized antibodies in order to reduce immunogenicity.

Furthermore, in the present invention, low-molecular-weight substances (low-molecular-weight compounds) that bind to the above-described CSPG core proteins, synthetases, desulfation enzyme-suppressing compounds, or sulfotransferases are also included in the substances capable of inhibiting the function of the above-described CSPG core proteins, synthetases, desulfation enzyme-suppressing compounds, or sulfotransferases. Such low-molecular-weight substances may be natural or artificial compounds. In general, the compounds can be produced or obtained by methods known to those skilled in the art. The compounds of the present invention can also be obtained by the screening methods described below.

In addition, the substances of the present invention capable of inhibiting the expression or function of the above-described CSPG core proteins, synthetases, desulfation enzyme-suppressing proteins, or sulfotransferases include dominant-negative mutants (dominant-negative proteins) for the above-described CSPG core proteins, synthetases, desulfation enzyme-suppressing proteins, or sulfotransferases. The “dominant-negative protein mutants for the above CSPG core proteins, synthetases, desulfation enzyme-suppressing proteins, or sulfotransferases” refer to proteins with the function of reducing or abolishing the activity of endogenous wild-type proteins by expressing the genes encoding the CSPG core proteins, synthetases, desulfation enzyme-suppressing proteins, or sulfotransferases. Such dominant-negative proteins include, for example, versican core protein mutants that competitively inhibit the linking of the wild-type versican core protein with chondroitin sulfate.

Furthermore, in the present invention, tissues and cells where the production or accumulation of chondroitin sulfate proteoglycans is inhibited are not particularly limited, but are preferably liver tissues.

Compounds that inhibit the production or accumulation of chondroitin sulfate proteoglycans are expected to serve as therapeutic or preventive agents for fibrotic liver diseases. Herein, “therapeutic or preventive” does not necessarily refer to a perfect therapeutic or preventive effect on tissues or cells with hepatic fibrosis, and may refer to a partial effect.

Herein, the fibrotic liver diseases are not particularly limited, as long as they are associated with hepatic fibrosis; however, they preferably include chronic liver diseases, such as chronic hepatitis, liver fibrosis, cirrhosis, liver failure, and hepatocarcinoma. In addition, complications of these diseases are also included in the fibrotic liver diseases of the present invention.

The hepatic fibrosis-suppressing agents of the present invention have an activity of suppressing hepatic fibrosis by inhibiting the production or accumulation of chondroitin sulfate proteoglycans which causes hepatic fibrosis. Thus, preferred embodiments of the present invention provide, for example, therapeutic agents for chronic liver diseases or cirrhosis, which comprise a hepatic fibrosis-suppressing agent of the present invention as an active ingredient.

The “hepatic fibrosis-suppressing agents” of the present invention can also be referred to as “therapeutic agents for hepatic fibrosis”, “hepatic fibrosis-improving agents”, or the like. Meanwhile, the “suppressing agents” of the present invention can also be referred to as “pharmaceutical agents”, “pharmaceutical compositions”, “therapeutic medicines”, or the like.

The “treatments” of the present invention also comprise preventive effects that can suppress the onset of hepatic fibrosis in advance. The treatments are not limited to those producing a perfect therapeutic effect on cells (tissues) developing hepatic fibrosis, and the effects may be partial.

The agents of the present invention can be combined with physiologically acceptable carriers, excipients, diluents and such, and orally or parenterally administered as pharmaceutical compositions. Oral agents may be in the form of granules, powders, tablets, capsules, solutions, emulsions, suspensions, or the like. The dosage forms of parenteral agents can be selected from injections, infusions, external preparations, inhalants, suppositories, and the like. Injections include preparations for subcutaneous, intramuscular, and intraperitoneal injections, and the like. The external preparations include nasal preparations, ointments, and such. Techniques for formulating the above-described dosage forms that contain the agents of the present invention as primary ingredients are known.

For example, tablets for oral administration can be produced by compressing and shaping the agents of the present invention in combination with excipients, disintegrants, binders, lubricants, and the like. Excipients commonly used include lactose, starch, mannitol, and the like. Commonly used disintegrants include calcium carbonate, carboxymethylcellulose calcium, and the like. Binders include gum arabic, carboxymethylcellulose, and polyvinylpyrrolidone. Known lubricants include talc, magnesium stearate, and such.

Known coatings can be applied to tablets comprising the agents of the present invention to prepare enteric coated formulations or for masking. Ethylcellulose, polyoxyethylene glycol, or such can be used as a coating agent.

Meanwhile, injections can be prepared by dissolving the agents of the present invention, which are chief ingredients, together with an appropriate dispersing agent, or dissolving or dispersing the agents in a dispersion medium. Both water-based and oil-based injections can be prepared, depending on the selection of dispersion medium. When preparing water-based injections, the dispersing agent is distilled water, physiological saline, Ringer's solution or such. For oil-based injections, any of the various vegetable oils, propylene glycols, or such is used as a dispersing agent. If required, a preservative such as paraben may be added at this time. Known isotonizing agents such as sodium chloride and glucose can also be added to the injections. In addition, soothing agents such as benzalkonium chloride and procaine hydrochloride can be added.

Alternatively, the agents of the present invention can be formed into solid, liquid, or semi-solid compositions to prepare external preparations. Such solid or liquid compositions can be prepared as the same compositions as described above and then used as external preparations. The semi-solid compositions can be prepared using an appropriate solvent, to which a thickener is added if required. Water, ethyl alcohol, polyethylene glycol, and the like can be used as the solvent. Commonly used thickeners are bentonite, polyvinyl alcohol, acrylic acid, methacrylic acid, polyvinylpyrrolidone, and the like. Preservatives such as benzalkonium chloride can be added to these compositions. Alternatively, suppositories can be prepared by combining the compositions with carriers, like oil bases such as cacao butter, or aqueous gel bases such as cellulose derivatives.

When the agents of the present invention are used as gene therapy agents, the agents may be directly administered by injection, or vectors carrying the nucleic acid may be administered. Such vectors include adenovirus vectors, adeno-associated virus vectors, herpes virus vectors, vaccinia virus vectors, retroviral vectors, and lentivirus vectors. These vectors allow efficient administration.

Alternatively, the agents of the present invention can be encapsulated into phospholipid vesicles such as liposomes, and then the vesicles can be administered. Vesicles carrying siRNAs or shRNAs are introduced into given cells by lipofection. The resulting cells are then systemically administered, for example, intravenously or intraarterially. The cells can also be locally administered into tissues or such with hepatic fibrosis. siRNAs exhibit a quite superior and specific post-transcriptional suppression effect in vitro; however, in vivo they are rapidly degraded due to serum nuclease activity. Thus, their limited time in vivo becomes problematic, and there is therefore demand for the development of optimized and effective delivery systems. As one example, Ochiya et al. have reported that atelocollagen, a bio-affinity material, is a highly suitable siRNA carrier because it has the activity of protecting nucleic acids from nucleases in the body when mixed with the nucleic acids to form a complex (Ochiya, T. et al, Nat. Med. 1999, 5, 707-710; Ochiya, T. et al, Curr. Gene Ther. 2001, 1, 31-52). However, the methods for introducing drugs of the present invention are not limited thereto.

The agents of the present invention are administered to mammals including humans at required (effective) doses, within a dose range considered to be safe. Ultimately, the doses of the agents of the present invention can be appropriately determined by medical practitioners or veterinarians after considering the dosage form and administration method, and the patient's age and weight, symptoms, and the like. For example, adenoviruses are administered once a day at a dose of about 10⁶ to 10¹³ viruses every one to eight weeks, although the doses vary depending on the age, sex, symptoms, administration route, administration frequency, and dosage form.

Commercially available gene transfer kits (for example: AdenoExpress™, Clontech) may be used to introduce siRNAs or shRNAs into target tissues or organs.

When the agents of the present invention are used, the type of disease and site to which the agents are applied are not particularly limited, as long as the disease develops hepatic fibrosis; for example, the agents are applied to chronic hepatitis, liver fibrosis, cirrhosis, liver failure, hepatocarcinoma, and such. The above diseases may occur in combination with other diseases.

In another preferred embodiment, the present invention relates to hepatic fibrosis-suppressing agents comprising chondroitinase ABC as an active ingredient.

Chondroitinase ABC is a lyase classified under enzyme number EC 4.2.2.4 (chondroitin ABC lyase). Chondroitinase ABC has an activity of removing polysaccharides that have 1,4-β-D-hexosaminide linkage, and 1,3-β-D-glucuronosyl linkage or 1,3-(α-L-iduronosyl linkage by degrading them to disaccharides with a 4-deoxy-β-D-gluc-4-enuronosyl group. Chondroitinase ABC acts on and degrades chondroitin sulfate A (chondroitin-4-sulfate), chondroitin sulfate B (dermatan sulfate), chondroitin sulfate C (chondroitin-6-sulfate), and hyaluronic acid.

Chondroitinase ABC used in the present invention preferably includes, for example, bacteria of the genus Proteus, such as Proteus vulgaris, and bacteria of the genus Bacteroides, such as Bacteroides stercoris, but is not limited thereto. More preferably, chondroitinase ABC used in the present invention is derived from Proteus vulgaris. Chondroitinase ABC of the present invention may be a sample purified from extracts of Proteus vulgaris by ammonium sulfate precipitation and DEAE-cellulose chromatography (Yamagata, T. et al., J. Biol. Chem. 1968, 243, 1523-1535). The Proteus vulgaris-derived chondroitinase ABC of the present invention includes, for example, proteins comprising the amino acid sequence of SEQ ID NO: 9 disclosed in Japanese Patent Application Kokai Publication No. (JP-A) H06-098769 (unexamined, published Japanese patent application) and proteins encoded by the nucleotide sequence deposited under Accession No. E07183 in the international nucleotide sequence database DDBJ/EMBL/GenBank (for example, by accessing the web site of the DNA Data Bank of Japan (DDBJ): http://www.ddbj.nig.ac jp/Welcome-j.html). It is preferred that chondroitinase ABC of the present invention at least has an activity of degrading chondroitin sulfate A (chondroitin-4-sulfate), chondroitin sulfate B (dermatan sulfate), chondroitin sulfate C (chondroitin-6-sulfate), and hyaluronic acid.

Commercially available products of chondroitinase ABC, for example, chondroitinase ABC (Proteus vulgaris, Seikagaku Co., Tokyo, Japan) and chondroitinase ABC (Proteus vulgaris, Sigma-Aldrich, Saint Louis, Mo., USA) can be used in the present invention. Alternatively, those skilled in the art can readily produce chondroitinase ABC of the present invention by expressing a protein from chondroitinase ABC-encoding DNA (for example, DNA comprising the nucleotide sequence deposited under Accession No. E07183) using recombinant techniques. Conventional molecular biology methods, such as recombinant techniques, are described in detail, for example, in Molecular Cloning: A Laboratory Manual/Volume 3, Joseph Sambrook and David W Russell. 3rd Ed., New York, Cold Spring Harbor Laboratory Press, 2001, and so on.

The hepatic fibrosis-suppressing agents of the present invention comprising chondroitinase ABC as an active ingredient can effectively suppress liver tissue fibrosis (hepatic fibrosis). Herein, “suppressing fibrosis” means reducing or eliminating fibrotic lesions in fibrotic tissues, or retarding or inhibiting further advancement of fibrosis (suppressing the expansion of fibrotic lesions).

The degree of fibrosis in liver tissues can be evaluated by various methods known to those skilled in the art. In the most fundamental method, fibrosis may be evaluated by histologically observing fibrotic findings in liver biopsy, for example, images of fibrotic tissues emphasized by special staining (aniline blue stain, trichrome stain, silver stain, and such) in liver biopsy samples. Specifically, evaluation of fibrosis can be achieved, for example, by presenting the degree of hepatic fibrosis in each sample measured by immunohistochemical staining in terms of a fibrosis score according to Dai K, et al., World J. Gactroenterol. 2005, 31, 4822-4826; or Hillebrandt S, et al., Nature Genetics 2005, 37, 835-843. Alternatively, the degree of hepatic fibrosis in liver tissues may be evaluated more simply by using hepatic fibrosis markers, such as hyaluronic acid, type I, III, and IV collagens and other collagens, fibroblasts, and macrophages. Simple tests that comprise determining the platelet count, which sensitively reflects the degree of hepatic fibrosis, can also be conveniently used. Alternatively, the degree of hepatic fibrosis can also be roughly estimated by image diagnosis of liver, such as abdominal ultrasonographic examination. Alternatively, the degree of hepatic fibrosis can also be evaluated using a measurement device (FibroScan 502, or such) for a non-invasive method that examines hepatic fibrosis based on the transient elastography technology recently developed by EchoSens (France). The degree of suppression of hepatic fibrosis by a hepatic fibrosis-suppressing agent of the present invention comprising as an active ingredient chondroitinase ABC may be determined based on evaluation of the degree of hepatic fibrosis by the methods described above.

The hepatic fibrosis-suppressing effect of hepatic fibrosis-suppressing agents of the present invention comprising chondroitinase ABC as an active ingredient is preferably accompanied by in particular, suppression of at least one of type III collagen elongation, type I collagen deposition, fibroblast accumulation, and macrophage accumulation in liver tissues, and more preferably suppression of all of them. The degree of suppression of hepatic fibrosis by the hepatic fibrosis-suppressing agents of the present invention comprising chondroitinase ABC as an active ingredient may be determined using one or more of the suppression effects described above as an indicator.

The present invention is based on a completely novel finding that chondroitinase ABC, which is an active ingredient in the hepatic fibrosis-suppressing agents of the present invention that comprise chondroitinase ABC as an active ingredient, produces a very strong hepatic fibrosis-suppressing effect, as demonstrated in the Examples described below. The hepatic fibrosis-suppressing effect of chondroitinase ABC is far superior when compared to other chondroitinases. This superior effect of chondroitinase ABC can be attributed to the fact that the type and quantity of chondroitin sulfate that is difficult to be cleaved by chondroitinase AC, chondroitinase B, or such accumulates in fibrotic liver tissues, and is for the first time successfully cleaved by only chondroitinase ABC. In recent years, various types of chondroitin sulfates have been reported, including chondroitin sulfates with varying degrees of sulfation and chondroitin sulfates having sulfate groups at different positions. Therefore, the hypothesis described above is highly convincing. However, the present invention is not limited to such hypothesis.

According to the present invention, the degree of fibrosis in liver tissues can be reduced by adding and reacting a hepatic fibrosis-suppressing agent of the present invention comprising chondroitinase ABC as an active ingredient to liver tissue samples.

According to the present invention, fibrosis of liver tissues in a subject can be effectively suppressed by administering a hepatic fibrosis-suppressing agent of the present invention comprising chondroitinase ABC as an active ingredient to the subject. There is no limitation on the administration route of the hepatic fibrosis-suppressing agents comprising chondroitinase ABC as an active ingredient; however, parenteral route is preferred. Preferred examples of such parenteral routes include intravenous, intraarterial, intraperitoneal, intrahepatic, intrarectal, and subcutaneous routes. The hepatic fibrosis-suppressing agents of the present invention comprising chondroitinase ABC as an active ingredient may be administered systemically or locally (for example, administered directly to fibrotic liver tissues).

The present invention also relates to methods for suppressing liver fibrosis in subjects, which comprise administering a hepatic fibrosis-suppressing agent of the present invention comprising chondroitinase ABC as an active ingredient.

Furthermore, the hepatic fibrosis-suppressing agents of the present invention comprising chondroitinase ABC as an active ingredient can effectively suppress liver tissue fibrosis, and thus can be used advantageously to treat and prevent fibrotic liver diseases (diseases with excessive fibrosis of liver tissues). In the present invention, more preferred examples of fibrotic liver diseases include, but are not limited to, chronic liver diseases. Chronic liver diseases generally involve hepatic fibrosis. Specifically, such chronic liver diseases of the present invention include, for example, chronic hepatitis (chronic hepatitis B, chronic hepatitis C, and such), liver fibrosis, cirrhosis, liver failure, and hepatocarcinoma. In particular, the hepatic fibrosis-suppressing agents of the present invention comprising chondroitinase ABC as an active ingredient are useful in treating and preventing cirrhosis. Herein, “cirrhosis” refers to a liver condition where the liver is extensively fibrotic and is altered into an aberrant hepatic lobular structure (pseudolobule/regenerating nodule) after losing its normal hepatic lobular structure. Cirrhosis has been divided into many types depending on the cause of disease, characteristic features of pseudolobule, and so on. Furthermore, a liver in such conditions is called a cirrhotic liver. Meanwhile, “liver fibrosis” is a condition showing aberrant fibrosis but no alteration of hepatic lobule, and is in general assumed to be a preliminary stage of cirrhosis. Specifically, cirrhosis of the present invention includes, but is not limited to, for example, viral cirrhosis, parasitic cirrhosis, toxic cirrhosis, trophopathic cirrhosis, alcoholic cirrhosis, cardiac cirrhosis, hepatic sclerosis, Charcot's cirrhosis, Todd's cirrhosis, primary biliary cirrhosis, secondary biliary cirrhosis, monolobular cirrhosis, cirrhosis resulting from chronic non-suppurative destructive cholangitis, obstructive cirrhosis, cholangiolitic cirrhosis, biliary cirrhosis, atrophic cirrhosis, postnecrotic cirrhosis, posthepatitic cirrhosis, nodular cirrhosis, mixed cirrhosis, micronodular cirrhosis, compensatory cirrhosis, non-compensatory cirrhosis, macronodular cirrhosis, septal cirrhosis, cryptogenic cirrhosis, periportal cirrhosis, and portal cirrhosis.

Preferred subjects to be administered with the hepatic fibrosis-suppressing agents of the present invention comprising chondroitinase ABC as an active ingredient are mammals, including humans, domestic animals, pets, and experimental animals. In particular, mammals (patients) with fibrotic liver diseases, for example, cirrhosis and liver fibrosis, are preferred subjects of the present invention. In another embodiment, mammals (patients) with fibrotic liver diseases that tend to progress to cirrhosis (for example, chronic non-suppurative destructive cholangitis) are also preferred subjects. The present invention reduces the degree of liver fibrosis, or retards or inhibits the progression of fibrosis by administering a hepatic fibrosis-suppressing agent of the present invention comprising chondroitinase ABC as an active ingredient to subjects. As a result, liver damages caused by fibrosis are reduced to improve the liver function or keep it stronger. Thus, the present invention can treat or prevent fibrotic liver diseases.

The dosage of the hepatic fibrosis-suppressing agents of the present invention comprising chondroitinase ABC as an active ingredient can be determined by those skilled in the art. In general, the dosage is equivalent to 10 μg to 1 mg chondroitinase ABC/1 kg body weight.

Thus, the present invention also relates to methods for treating or preventing fibrotic liver diseases (in particular, cirrhosis and such), including chronic liver diseases, which comprise administering the hepatic fibrosis-suppressing agents of the present invention comprising chondroitinase ABC as an active ingredient to subjects (for example, patients).

The hepatic fibrosis-suppressing agents of the present invention comprising chondroitinase ABC as an active ingredient may be pharmaceutical compositions appropriately comprising pharmaceutically acceptable (pharmaceutically inactive) carriers, excipients, and/or diluents, in addition to chondroitinase ABC. Such carriers, excipients, and/or diluents include, but are not limited to, for example, water, physiological saline, phosphate buffered saline, polyvinyl alcohol, polyvinylpyrrolidone, carboxyvinyl polymer, sodium alginate, water-soluble dextran, pectin, xanthan gum, gum Arabic, gelatin, agar, glycerin, propylene glycol, polyethylene glycol, vaseline, paraffin, stearyl alcohol, stearic acid, human serum albumin, mannitol, sorbitol, and lactose. The pharmaceutical compositions of the present invention may further comprise additives, such as preservatives. The pharmaceutical compositions of the present invention may further comprise other pharmacological ingredients.

The pharmaceutical composition of the present invention may be provided as oral or parenteral preparations; however they are preferably provided as parenteral preparations. Preferred parenteral preparations are liquid preparations such as liquids and suspensions. Injections are especially preferred.

The present invention also provides methods of screening for hepatic fibrosis-suppressing agents, wherein the methods comprise selecting, from test samples, substances with the activity of inhibiting the production or accumulation of chondroitin sulfate proteoglycans. Hepatic fibrosis-suppressing agents or candidate compounds for hepatic fibrosis-suppressing agents can be efficiently obtained using the screening methods of the present invention.

Preferred embodiments of the screening methods of the present invention are methods of screening for hepatic fibrosis-suppressing agents that comprise the step of selecting substances with any of the activities (a) to (d):

(a) promoting the degradation of chondroitin sulfate proteoglycans;
(b) inhibiting the synthesis of chondroitin sulfate proteoglycans;
(c) desulfating chondroitin sulfate proteoglycans; and
(d) inhibiting the sulfation of chondroitin sulfate proteoglycans.

Representative examples based on fundamental principles common in screening for these substances include methods comprising the following procedure:

A preferred procedure uses tools (1) to (3) below, and is as follows: (1) and (2) are mixed in a test tube or culture dish, and the resulting effect is simply detected using (3).
(1) chondroitin sulfate proteoglycans (CSPGs) themselves, or glycosaminoglycans (GAG) chains, or cells synthesizing (producing) CSPGs or GAG chains
(2) test compounds (for example, enormous compound libraries owned by pharmaceutical companies)
(3) methods for detecting CSPG cleavage sites, the amount of CSPGs, or the amount of free glycosaminoglycans (GAGs)

Embodiments of the screening methods of the present invention are exemplified below. In the embodiments described below, the chondroitin sulfate proteoglycans, synthetases, compounds suppressing desulfation enzymes, sulfotransferases, degradation-promoting enzymes, and desulfation enzymes to be used include those derived from humans, mice, rats, and others, but are not limited thereto. Chondroitin sulfate proteoglycan portions are components such as glycosaminoglycan chains or core proteins, or portions thereof. The chondroitin sulfate proteoglycan portions are not particularly limited.

The test compounds to be used in the embodiments described below are not particularly limited, but include, for example, single compounds, such as natural compounds, organic compounds, inorganic compounds, proteins, and peptides, as well as compound libraries, expression products of gene libraries, cell extracts, cell culture supernatants, products of fermenting microorganisms, extracts of marine organisms, and plant extracts.

In the embodiments described below, the “contact” with test compounds is typically achieved by mixing the test compounds with chondroitin sulfate proteoglycans, portions thereof, synthetases, compounds suppressing desulfation enzymes, sulfotransferases, degradation-promoting enzymes, or desulfation enzymes, but the “contact” is not limited to this methods. For example, the “contact” can also be achieved by contacting test compounds with cells expressing these proteins or portions thereof.

In the embodiments described below, the “cells” include those derived from humans, mice, rats, and such, but are not limited thereto. Cells of microorganisms, such as Escherichia coli and yeasts, which are transformed to express the proteins used in each embodiment, can also be used. For example, the “cells that express chondroitin sulfate proteoglycans” include cells that express endogenous genes for chondroitin sulfate proteoglycans, and cells that express introduced foreign genes for chondroitin sulfate proteoglycans. Such cells that express foreign genes for chondroitin sulfate proteoglycans can typically be prepared by introducing host cells with expression vectors carrying a chondroitin sulfate proteoglycan gene as an insert. The expression vectors can be prepared using standard genetic engineering techniques.

The “chondroitin sulfate proteoglycan core proteins” described below include, for example, core proteins of matrix-type chondroitin sulfate proteoglycans, such as aggrecan, versican, neurocan, and brevican, and core proteins of membrane chondroitin sulfate proteoglycans, such as decorin, biglycan, fibromodulin, and PG-Lb. The “synthetases” include, for example, GalNAc4ST-1, GalNAc4ST-2, GALNAC4S-6ST, UA2OST, GalT-I, GalT-II, GlcAT-I, and XylosylT. The “sulfotransferases” include, for example, chondroitin D-N-acetylgalactosamine-4-O-sulfotransferase 1 (C4ST-1), chondroitin D-N-acetylgalactosamine-4-O-sulfotransferase 2 (C4ST-2), chondroitin D-N-acetylgalactosamine-4-O-sulfotransferase 3 (C4ST-3), D4ST, C6ST-1, and C6ST-2. The “degradation-promoting enzymes” include, for example, ADAMTS-1, ADAMTS-4, ADAMTS-5, chondroitinase ABC (ChABC), chondroitinase AC, chondroitinase B, and calpain I. The “desulfation enzymes” include, for example, chondroitin-4-sulfatase and chondroitin-6-sulfatase.

Embodiments of the screening methods of the present invention include methods comprising the step of selecting compounds that have the activity of promoting the degradation of chondroitin sulfate proteoglycans. An example of the above-mentioned methods of the present invention comprises the steps of:

(a) contacting test compounds with chondroitin sulfate proteoglycans or portions thereof;
(b) measuring the abundance of chondroitin sulfate proteoglycans or portions thereof; and
(c) selecting substances that reduce the abundances as compared with those determined in the absence of the test compounds.

In the above methods, first, test compounds are contacted with chondroitin sulfate proteoglycans or portions thereof.

In these methods, the amount of the chondroitin sulfate proteoglycans or portions thereof is then measured. The measurement can be conducted by methods known to those skilled in the art. For example, the amounts can be detected using labeled compounds or antibodies that bind to the chondroitin sulfate proteoglycans or portions thereof, and then measuring the amount of the label. Alternatively, the detection can be achieved by chromatography or mass spectrometry.

In these methods, compounds that reduce the abundance of the chondroitin sulfate proteoglycans or portions thereof as compared with in the absence of a test compound (the control) are then selected. Compounds resulting in a reduction can be used as therapeutic agents for hepatic fibrosis.

Below is a brief illustrative example of the methods able to assess (measure) whether a test compound has the above activity (a): the activity of promoting the degradation of chondroitin sulfate proteoglycans.

### Embodiment of the Methods for Screening for the Above Activity (a) of Promoting the Degradation of Chondroitin Sulfate Proteoglycans:

A CS-GAC, such as chondroitin sulfate A (CS-A), CS-B, CS-C (Seikagaku Co., ICN, Sigma, and others), or human-derived proteoglycan (BGN, ISL, and others), is prepared, and 96-well plates are coated with it at a concentration of 10 μg/ml (using known methods, such as in Kawashima, H. et al., J. Biol. Chem. 2002, 277, 12921-12930). Various test compounds are added to each well of the plates. After two hours of reaction at 37° C., changes in CS-GAG are detected.

Detection methods include, for example, the simple WFA lectin (Wisteria floribunda lectin)-binding method. Since WFA lectin binds to the GalNAc residues of CS-GAG chains, it can easily detect CS-GAGs. Chondroitinase ABC is used as a positive control for test compounds. The principle behind this use of chondroitinase ABC is that its addition degrades CS-GAG chains, making it impossible for WFA lectin to bind them. More specifically, FITC-labeled WFA lectin (EY Co.) is added to the CS-coated wells before and after mixing the test compounds, and changes in the intensity of FITC fluorescence in the wells due to the CS-GAG degradation can be quantified and digitized very simply by using detection devices, such as fluorescence plate readers or fluorescence microscopes. Compounds whose addition most reduces fluorescent values may be determined to be novel therapeutic candidate compounds that fulfill the concept of the present invention.

In an alternative detection method, anti-CS antibody (clone CS56, Seikagaku Co.) can be used to directly label CS-GAGs. As with WFA lectin, large-scale screening can be carried out simply and in very short time by adding FITC-labeled anti-CS antibody to CS-coated wells and examining changes in fluorescence value.

In more specific detection methods, GAG content is accurately quantified and digitized by simply using the plates before and after mixing of test compounds in an sGAG Assay Kit (Wieslab Co.), an ELISA Kit for Sulphanated Glycosaminoglycans (Funakoshi Co.), or such.

More specifically, the reducing ends of free GAG chains can easily be fluorescently labeled by adding 2-aminobenzamide, 2-aminopyridine (2-AB and 2-AP, respectively; LUD Co. and others), or the like to the plates before and after mixing of test compounds, which enables more specific analysis using HPLC, MALDI-MS, LC-MS, or such to determine the types of sugar chains and even the content of each type of chain. These methods, which examine the properties of candidate compounds in detail, take screening to the next level.

Other embodiments of the screening methods of the present invention include methods comprising the step of selecting substances with the activity of inhibiting the synthesis of chondroitin sulfate proteoglycans. These methods of the present invention comprise, for example, the steps of:

(a) contacting test compounds with cells expressing chondroitin sulfate proteoglycans or portions thereof, extracts of these cells, or groups of substances including those enzymes and substrates constituting the process of chondroitin sulfate proteoglycan synthesis;
(b) measuring the amount of synthesized chondroitin sulfate proteoglycans or intermediates thereof in the above-mentioned cells, cell extracts, or group of substances; and
(c) selecting compounds that reduce the amount as compared to in the absence of the test compounds.

In the above methods, test compounds are first contacted with cells expressing chondroitin sulfate proteoglycans or portions thereof, extract of these cells, or groups of substances including those enzymes and substrates that constitute the process of chondroitin sulfate proteoglycan synthesis.

Next, the amount of synthesized chondroitin sulfate proteoglycans or intermediates thereof is measured. The measurement can be performed by those skilled in the art using known methods; for example, methods using labeled antibodies, mass spectrometry, and chromatography can be used.

Further, compounds that reduce (suppress) the synthesized amount as compared with in the absence of the test compounds (the control) are selected. Compounds resulting in a reduction (suppression) can be used as therapeutic agents for hepatic fibrosis.

Below is a brief illustrative example of the methods able to assess (measure) whether a test compound has the above activity (b): the activity of inhibiting the synthesis of chondroitin sulfate proteoglycans.

### Embodiment of the Methods for Screening for the Above Activity (b) of Inhibiting the Synthesis of Chondroitin Sulfate Proteoglycans:

Cells and cell lines synthesizing chondroitin sulfate are known to researchers in the art. In human, for example, chondroitin sulfate is produced after 16 hours of cell culture by standard methods for culturing mononuclear cells isolated from peripheral blood collected from healthy subjects (Uhlin-Hansen, L. et al., Blood 1993, 82, 2880; etc.). Alternatively, for more convenience, there are many examples of known cell; for example, the fibroblast cell line NIH3T3 (Phillip, H. A. et al., J. Biol. Chem. 2004, 279, 48640; etc.); the renal tubule-derived cancer cell line ACHN (Kawashima, H. et al., J. Biol. Chem. 2002, 277, 12921), the renal distal tubule-derived cell line MDCK (Borges, F. T. et al., Kidney Int. 2005, 68, 1630; etc.), and the vascular endothelial cell line HUVEC (Schick, B. P. et al., Blood 2001, 97, 449; etc.). Various test compounds are added during the process of culturing such cell lines for set periods, and changes in the amount of CS-GAG before and after culture can be easily evaluated by the above-described method of (a). Compounds that suppress the increase in the amount of CS-GAG after culture (which thus reflects the amount of synthesized CS-GAG) can be easily determined to be candidate therapeutic compounds that fulfill the concept of the present invention.

As a further option, cell lines constitutively expressing the genes for CS-GAG synthetases such as GalNAc4ST-1 and XylosylT can be prepared by introducing the genes into CHO cells, L cells, or such by well-known methods. The use of such cell lines that constitutively synthesize CS-GAG allows more clear determination of candidates for therapeutic compounds.

In another embodiment, the screening methods of the present invention include methods comprising the step of selecting substances with the activity of desulfating chondroitin sulfate proteoglycans. The above methods of the present invention comprise, for example, the steps of:

(a) contacting test compounds with chondroitin sulfate proteoglycans or portions thereof;
(b) measuring the amount of sulfation in the chondroitin sulfate proteoglycans or portions thereof, and
(c) selecting substances that reduce the amount of sulfation as compared with in the absence of the test compounds.

In the above methods, test compounds are first contacted with chondroitin sulfate proteoglycans or portions thereof.

Next, the amount of sulfation in the chondroitin sulfate proteoglycans or portions thereof is measured. The measurement can be conducted using methods known to those skilled in the art. For example, the amount of sulfation can be determined by using labeled compounds or antibodies that bind to the desulfated structures remaining in the chondroitin sulfate proteoglycans or portions thereof, and measuring the amount of the label. Alternatively, the measurement can be achieved by chromatography or mass spectrometry or such.

Then, in the present methods compounds that reduce the abundances of the chondroitin sulfate proteoglycans or portions thereof as compared with in the absence of the test compounds (the control) are selected. Compounds resulting in a reduction can be used as therapeutic agents for hepatic fibrosis.

Below is a brief illustrative example of the methods able to assess (measure) whether a test compound has the above activity (c): the activity of desulfating chondroitin sulfate proteoglycans.

### Embodiment of the Methods for Screening for the Above Activity (c) of Desulfating Chondroitin Sulfate Proteoglycans:

By using essentially the same method as described above in (a), human-derived proteoglycans (BGN Co., ISL Co., etc.) or such are prepared and coated on to 6-well plates at a concentration of 10 μg/ml (by known methods, such as those described in Kawashima, H. et al., J. Biol. Chem. 2002, 277, 12921-12930). Various test compounds are added to each well of the plates, and alterations in CS-GAG are detected after two hours of reaction at 37° C.

In this detection method, desulfated moieties can be easily detected using the reaction of either anti-proteoglycan Δdi4S antibody (clone: 2-B-6, which recognizes sulfated moieties at position 4) or anti-proteoglycan Δdi6S antibody (clone: 3-B-3, which recognizes sulfated moieties at position 6) (both from Seikagaku Co.) with the disaccharide structure of the desulfated fragments that remain in the core protein of proteoglycans after desulfation. Thus, FITC-labeled 2-B-6 or 3-B-3 antibody is reacted in such plates before and after mixed culture, and changes in the fluorescence value can be simply detected. Compounds whose addition increases the fluorescence intensity can be determined to be substances that promote desulfation, and are easily identified as novel candidate therapeutic compounds that fulfill the concept of the present invention.

Another embodiment of the screening methods of present invention includes methods comprising the step of selecting substances with the activity of inhibiting the sulfation of chondroitin sulfate proteoglycans. The above methods of the present invention comprise, for example, the steps of:

(a) contacting test compounds with cells expressing chondroitin sulfate proteoglycans or portions thereof, extracts of these cells, or groups of substances including those enzymes and substrates constituting the process of sulfation of chondroitin sulfate proteoglycans;
(b) measuring the activity of sulfation of chondroitin sulfate proteoglycans in the above-mentioned cells, cell extracts, or groups of substances; and
(c) selecting compounds that reduce the activity as compared with in the absence of the test compounds.

In the above methods, test compounds are first contacted with chondroitin sulfate proteoglycans or portions thereof.

Next, the amount of sulfation in the chondroitin sulfate proteoglycans or portions thereof is measured. The measurement can be conducted using methods known to those skilled in the art. For example, the amount of sulfation can be determined by using labeled compounds or antibodies that bind to the sulfated structures of the chondroitin sulfate proteoglycans or portions thereof, and measuring the amount of the label. Alternatively, the measurement can be achieved by chromatography or mass spectrometry and such.

Then, compounds that reduce the abundance of the chondroitin sulfate proteoglycans or portions thereof as compared with in the absence of the test compounds (the control) are selected. Compounds resulting in a reduction can be used as therapeutic agents for hepatic fibrosis.

Below is a brief illustrative example of the methods able to assess (measure) whether a test compound has the above activity (d): the activity of inhibiting the sulfation of chondroitin sulfate proteoglycans.

### Embodiment of the Methods for Screening for the Above Activity (d) of Inhibiting the Sulfation of Chondroitin Sulfate Proteoglycans:

The cells and cell lines that promote the sulfation of chondroitin sulfates are the same as described above in (c). Various test compounds are mixed during a set period of culture of such cell lines, and the degree of sulfation before and after the culture can be easily determined by, for example, using an antibody that recognizes sulfation at position 4 (clone: LY111) or an antibody that recognizes sulfation at position 6 (clone: MC21C) (both from Seikagaku Co.). Fluorescence values may be compared between before and after the culture by using fluorescently labeled antibodies. Alternatively, the same detection method as described above in (c) can be conducted using 2-B-6 or 3-B-3 antibodies before and after culture. Compounds that suppress an increase in the sulfation after cell culture (an increase in the fluorescence value for LY111 or MC21C), or compounds that promote the progression of desulfation after cell culture (an increase in the fluorescence value for 2-B-6 or 3-B-3) can be easily determined to be candidate therapeutic compounds that fulfill the concept of the present invention.

As a further option, cell lines that constitutively express sulfotransferase genes such as C4ST-1 and C6ST-1 can be prepared by introducing the genes into CHO cells, L cells, or such by well-known methods. The use of such cell lines that constitutively add sulfate groups allows more clear determination of candidates for therapeutic compounds.

Other preferred embodiments of the present invention are methods of screening for hepatic fibrosis-suppressing agents in which compounds that reduce the expression level of a gene encoding a CSPG core protein, synthetase, desulfation enzyme-suppressing protein, or sulfotransferase of the present invention, or compounds that increase the expression level of a gene for an enzyme that desulfates CSPGs or promotes the degradation of CSPGs, are selected; wherein the method comprises the steps of:

(a) contacting test compounds with cells expressing a gene encoding a CSPG core protein, synthetase, desulfation enzyme-suppressing protein, sulfotransferase, degradation-promoting enzyme, or desulfating enzyme;
(b) determining the expression level of the gene in the cells;
(c) comparing the expression level with that in the absence of the test compounds (the control); and
(d) selecting compounds that reduce the expression level of the gene of the CSPG core protein, synthetase, desulfation enzyme-suppressing protein, or sulfotransferase as compared with the control, or compounds that increase the expression level of the gene of the CSPG desulfating enzyme or the CSPG degradation-promoting enzyme as compared with the control.

In the above methods, test compounds are first contacted with cells expressing a gene encoding a CSPG core protein, synthetase, desulfating enzyme-suppressing protein, sulfotransferase, degradation-promoting enzyme, or desulfating enzyme.

Next, the expression level of the gene encoding the core protein, synthetase, desulfating enzyme-suppressing protein, sulfotransferase, degradation-promoting enzyme, or desulfating enzyme is measured. Herein, “expression of the gene” includes both transcription and translation. Gene expression level can be measured by methods known to those skilled in the art.

For example, mRNAs are extracted from cells expressing any one of the above-described proteins by conventional methods, and these mRNAs can be used as templates in Northern hybridization, RT-PCR, DNA arrays, or such to measure the transcription level of the gene. Alternatively, protein fractions are collected from cells expressing a gene encoding any of the above-described proteins, and expression of the protein can be detected by electrophoresis such as SDS-PAGE to measure the level of gene translation. Alternatively, the level of gene translation can be measured by detecting the expression of any of the above-described proteins by Western blotting using an antibody against the proteins. Such antibodies for use in detecting the proteins are not particularly limited, as long as they are detectable. For example, both monoclonal and polyclonal antibodies can be used.

Next, the expression level is compared with that in the absence of the test compounds (the control).

Then, when the gene encodes a CSPG core protein, synthetase, desulfating enzyme-suppressing protein, or sulfotransferase, compounds that reduce (suppress) the expression level of the gene as compared with a control are selected. The compounds resulting in a reduction (suppression) can be agents for suppressing hepatic fibrosis or candidate compounds for treating hepatic fibrosis.

Alternatively, when the gene encodes a CSPG desulfating enzyme or an enzyme promoting CSPG degradation, compounds that increase (enhance) the expression level of the gene as compared with a control are selected. Compounds resulting in an increase (enhancement) can be agents for suppressing hepatic fibrosis or candidate compounds for treating hepatic fibrosis.

An embodiment of the screening methods of the present invention is a method in which compounds that reduce the expression level of a gene encoding a CSPG core protein, synthetase, desulfation enzyme-suppressing protein, or sulfotransferase of the present invention, or compounds that increase the expression level of a gene for a CSPG degradation-promoting enzyme or a CSPG desulfating enzyme, can be selected using the expression of a reporter gene as an indicator. The above methods of the present invention comprise, for example, the steps of:

(a) contacting test compounds with cells or cell extracts containing a DNA structured such that a reporter gene is operably linked to a transcriptional regulatory region of a gene encoding a CSPG core protein, synthetase, desulfating enzyme-suppressing protein, sulfotransferase, degradation-promoting enzyme, or desulfating enzyme;
(b) measuring the expression level of the reporter gene;
(c) comparing the level with the that in the absence of the test compounds (the control); and
(d) selecting compounds that reduce the expression level of the reporter gene as compared with the control when the reporter gene is operably linked with a transcriptional regulatory region of the gene encoding a CSPG core protein, synthetase, desulfating enzyme-suppressing protein, or sulfotransferase; or selecting compounds that increase the expression level of the reporter gene as compared with the control when the reporter gene is operably linked with a transcriptional regulatory region of a gene encoding a CSPG degradation-promoting enzyme or a CSPG desulfating enzyme.

In the above methods, test compounds are first contacted with cells or cell extracts containing DNAs structured such that a reporter gene is operably linked with a transcriptional regulatory region of a gene encoding a CSPG core protein, synthetase, desulfating enzyme-suppressing protein, sulfotransferase, degradation-promoting enzyme, or desulfating enzyme.

Herein, “operably linked” means that a reporter gene is linked with a transcriptional regulatory region of a gene encoding a CSPG core protein, synthetase, desulfating enzyme-suppressing protein, sulfotransferase, degradation-promoting enzyme, or desulfating enzyme, such that expression of the reporter gene is induced upon binding of transcriptional factors to the transcriptional regulatory region. Therefore, the meaning of “operably linked” also includes cases where a reporter gene is linked with a different gene and produces a fusion protein with a different gene product, as long as expression of the fusion protein is induced upon the binding of transcriptional factors to the transcriptional regulatory region of the gene encoding the CSPG core protein, synthetase, desulfating enzyme-suppressing protein, sulfotransferase, degradation-promoting enzyme, or desulfating enzyme. Those skilled in the art can obtain the transcriptional regulatory regions of genes encoding CSPG core proteins, synthetases, desulfating enzyme-suppressing proteins, sulfotransferases, degradation-promoting enzymes, or desulfating enzymes that are present in the genome, based on the cDNA nucleotide sequences of the genes encoding the CSPG core proteins, synthetases, desulfating enzyme-suppressing proteins, sulfotransferases, degradation-promoting enzymes, or desulfating enzymes.

The reporter genes for use in these methods are not particularly limited, as long as their expression is detectable. The reporter genes include, for example, the CAT gene, the lacZ gene, the luciferase gene, and the GFP gene. The “cells containing a DNA structured such that a reporter gene is operably linked with a transcriptional regulatory region of a gene encoding a CSPG core protein, synthetase, desulfating enzyme-suppressing protein, sulfotransferase, degradation-promoting enzyme, or desulfating enzyme” include, for example, cells introduced with vectors carrying such structures as inserts. Such vectors can be prepared by methods well known to those skilled in the art. The vectors can be introduced into cells by standard methods, for example, calcium phosphate precipitation, electroporation, lipofection, and microinjection. The “cells containing a DNA structured such that a reporter gene is operably linked with a transcriptional regulatory region of a gene encoding a CSPG core protein, synthetase, desulfating enzyme-suppressing protein, sulfotransferase, degradation-promoting enzyme, or desulfating enzyme” include cells in which the structure has been integrated into the chromosomes. A DNA structure can be integrated into chromosomes by methods generally used by those skilled in the art, for example, gene transfer methods using homologous recombination.

The “cell extracts containing a DNA structured such that a reporter gene is operably linked with a transcriptional regulatory region of a gene encoding a CSPG core protein, synthetase, desulfating enzyme-suppressing protein, sulfotransferase, degradation-promoting enzyme, or desulfating enzyme” include, for example, mixtures of cell extracts included in commercially available in vitro transcription-translation kits and DNAs structured such that a reporter gene is operably linked with the transcriptional regulatory region of the gene encoding a CSPG core protein, synthetase, desulfating enzyme-suppressing protein, or sulfotransferase.

“Contact” can be achieved by adding test compounds to a culture medium of “cells containing a DNA structured such that a reporter gene is operably linked with a transcriptional regulatory region of a gene encoding a CSPG core protein, synthetase, desulfating enzyme-suppressing protein, sulfotransferase, degradation-promoting enzyme, or desulfating enzyme”, or by adding test compounds to the above-described commercially available cell extracts containing the DNAs. When the test compound is a protein, contact may also be achieved, for example, by introducing a DNA vector expressing the protein into the cells.

In the above methods, the expression level of the reporter gene is then measured. The expression level of the reporter gene can be measured by methods known to those skilled in the art, depending on the type of the reporter gene. When the reporter gene is the CAT gene, its expression can be determined, for example, by detecting the acetylation of chloramphenicol by the gene product. When the reporter gene is the lacZ gene, its expression level can be determined by detecting the color development of chromogenic compounds due to the catalytic action of the gene expression product. Alternatively, when the reporter gene is the luciferase gene, its expression level can be determined by detecting the fluorescence of fluorogenic compounds due to the catalytic action of the gene expression product. Furthermore, when the reporter gene is the GFP gene, its expression level can be determined by detecting the fluorescence of the GFP protein.

In the above methods, the expression level of the reporter gene is then compared with that in the absence of the test compounds (the control).

In the present methods, compounds that reduce (suppress) the expression level of a reporter gene as compared with a control are then selected, where the reporter gene is operably linked with a gene encoding a CSPG core protein, synthetase, desulfating enzyme-suppressing protein, or sulfotransferase. Compounds resulting in a reduction (suppression) can be agents for suppressing hepatic fibrosis or candidate compounds for treating hepatic fibrosis.

Alternatively, when the reporter gene is operably linked with a gene encoding a CSPG degradation-promoting enzyme or CSPG desulfating enzyme, compounds that increase (enhance) the reporter gene expression level as compared with a control are selected. Compounds resulting in an increase (enhancement) can be agents for suppressing hepatic fibrosis or candidate compounds for treating hepatic fibrosis.

The hepatic fibrosis-suppressing agents that are found by the screening methods of the present invention are preferably therapeutic or preventive agents for fibrotic liver diseases.

The present invention also provides kits comprising various agents, reagents, and the like, which are used to conduct the screening methods of the present invention.

The kits of the present invention can be prepared, for example, by selecting adequate reagents from the above-described various reagents, depending on the screening method to be conducted. The kits of the present invention may contain, for example, the chondroitin sulfate proteoglycans of the present invention. The kits of the present invention may further contain various reagents, vessels, and the like to be used in the methods of the present invention. The kits may appropriately contain, for example, anti-chondroitin sulfate proteoglycan antibodies, probes, various reaction reagents, cells, culture media, control samples, buffers, and instruction manuals containing a description of how to use the kits.

Preferred embodiments of the present invention are the methods of screening for hepatic fibrosis-suppressing agents, comprising the step of detecting whether the production or accumulation of chondroitin sulfate proteoglycans is inhibited. Thus, the kits for screening for the hepatic fibrosis-suppressing agents of the present invention may contain, for example, oligonucleotides such as probes for the genes encoding CSPG core proteins, and primers to amplify certain regions of these genes; and antibodies recognizing CSPGs (anti-chondroitin sulfate proteoglycan antibodies), which can be used to detect chondroitin sulfate proteoglycans.

The above-described oligonucleotides specifically hybridize to, for example, DNAs of the genes encoding the versican core protein of the present invention. Herein, “specifically hybridize to” means that the oligonucleotides do not significantly cross-hybridize to DNAs encoding other proteins under standard hybridization conditions, and preferably under stringent hybridization conditions (for example, the conditions described in Sambrook, J. et al. “Molecular Cloning” 2nd Ed., Cold Spring Harbour Laboratory Press, New York, USA, 1989). The oligonucleotides are not necessarily perfectly complementary to the nucleotide sequences of the versican core protein genes of the present invention, as long as they allow specific hybridization.

The hybridization conditions in the present invention include, for example, conditions such as “2×SSC, 0.1% SDS, and 50° C.”, “2×SSC, 0.1% SDS, and 42° C.”, and “1×SSC, 0.1% SDS, and 37° C.”, and more stringent conditions such as “2×SSC, 0.1% SDS, and 65° C.”, “0.5×SSC, 0.1% SDS, and 42° C.”, and “0.2×SSC, 0.1% SDS, and 65° C.”. More specifically, for methods using Rapid-hyb buffer (Amersham Life Science), prehybridization is carried out at 68° C. for 30 minutes or more; then a probe is added, and after one hour or more of hybrid formation at 68° C., washing is carried out three times with 2×SSC/0.1% SDS at room temperature for 20 minutes, then three times with 1×SSC/0.1% SDS at 37° C. for 20 minutes, and finally twice with 1×SSC/0.1% SDS at 50° C. for 20 minutes. Alternatively, for example, prehybridization is carried out in Expresshyb Hybridization Solution (CLONTECH) at 55° C. for 30 minutes, and then a labeled probe is added thereto, and after one hour or more of incubation at 37-55° C., washing is carried out three times in 2×SSC/0.1% SDS at room temperature for 20 minutes and then once in 1×SSC/0.1% SDS at 37° C. for 20 minutes. More stringent conditions can be achieved, for example, by increasing the temperature for prehybridization, hybridization, or second washing. For example, temperatures for prehybridization and hybridization can be 60° C., and more stringent conditions can be achieved by increasing the temperature to 68° C. In addition to conditions such as the salt concentration of the buffers and the temperature, those skilled in the art can also determine conditions using other conditions including the nucleotide sequence composition of the probe, the probe length and concentration, and reaction time.

The oligonucleotides can be used as probes and primers in the above-described screening kits of the present invention. When the oligonucleotides are used as primers, they are typically 15 to 100 bp long, and preferably 17 to 30 bp long. Primers are not particularly limited as long as they can amplify at least a portion of a DNA of the above-described genes of the present invention.

The present invention also provides therapeutic or preventive methods for fibrotic liver diseases, which comprise the step of administering the agents of the present invention to individuals (for example, to patients and such).

The individuals subjected to the therapeutic or preventive methods of the present invention are not particularly limited, as long as they are organisms that can develop a fibrotic liver disease; however, humans are preferred.

In general, administration to individuals can be achieved, for example, by methods known to those skilled in the art, such as intraarterial injections, intravenous injections, and subcutaneous injections. The administered dose varies depending on the patient's weight and age, and the administration method or such; however, those skilled in the art (medical practitioners, veterinarians, pharmacists, and the like) can appropriately select a suitable dose.

The present invention also relates to the uses of agents of the present invention in producing hepatic fibrosis-suppressing agents.

All prior-art documents cited herein are incorporated by reference herein.

### EXAMPLES

Herein below, the present invention will be specifically described with reference to Examples, but the technical scope of the present invention is not to be construed as being limited thereto.

### Example 1

### Increase in the Expression Level of Chondroitin Sulfate Proteoglycans (CSPGs) in Cirrhosis Mouse Models

In this Example, a cirrhosis mouse model was prepared using a mouse liver fibrosis model induced by carbon tetrachloride (CCl₄), which is a typical example of mouse model for chronic fibrotic liver diseases, and is examined for the accumulation of proteoglycans using immunohistochemical staining. This liver fibrosis mouse model has superior reproducibility and is widely used as an experimental cirrhosis model (Sakaida I, et al., Hepatology 2004, 40, 1304-1311).

First, carbon tetrachloride (25 μl/100 g body weight, Sigma-Aldrich) was injected into the peritoneal cavities of C57BL/6JcL mice (female, five to six weeks old, CLEA Japan Inc.) twice a week for four weeks (eight times) to induce liver fibrosis. Then, carbon tetrachloride was additionally administered twice a week for two weeks (12 times in total) to induce cirrhosis. Mice with induced cirrhosis were sacrificed, and their livers were collected (cirrhotic livers). Meanwhile, in the control experiment, livers were collected from C57BL/6JcL mice (female, CLEA Japan Inc.) of the same age not administered with carbon tetrachloride (normal livers).

Portions of second lobes from the collected livers were embedded in the OCT compound (Miles), an embedding medium for cryosectioning, and cryoblocks were prepared using liquid nitrogen. The cryoblocks were sliced into 6-μm sections using cryostat (Microm).

The resulting sections were fixed with acetone (Wako Pure Chemical Industries Ltd.) for ten minutes, and then washed with phosphate buffer. Then, as a primary antibody, an anti-chondroitin sulfate proteoglycan (CSPG) antibody (clone CS56, mouse monoclonal antibody, 10 μg/ml, Seikagaku Co.), anti-chondroitin-4-sulfate proteoglycan (C4SPG) antibody (clone LY11, mouse monoclonal antibody, 10 μg/ml, Seikagaku Co.), anti-chondroitin-6-sulfate proteoglycan (C6SPG) antibody (clone MC21C, mouse monoclonal antibody, 10 μg/ml, Seikagaku Co.), or anti-heparan sulfate proteoglycan (HSPG)/Perlecan antibody (rat monoclonal antibody, 10 μg/ml, Dainippon Pharma.) was added, and the sections were incubated at room temperature for one hour. Next, the secondary antibody reaction was conducted using a Histofine Mouse Stain Kit (Nichirei, used for mouse monoclonal antibodies) or a peroxidase-labeled anti-rat IgG (Biosource, at 1:200 dilution, used for rat monoclonal antibodies), and then DAB substrate (Nichirei) was added thereto. The samples were observed under a light microscope (Leica Microsystems). Brown signals showing visible antibody binding were confirmed.

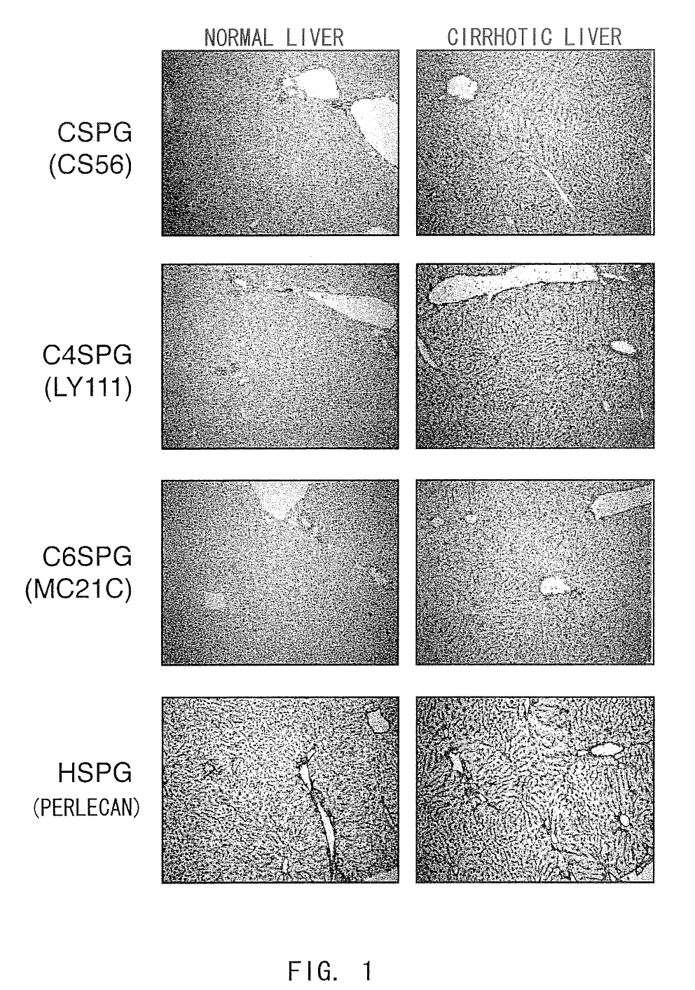

Examples of images obtained by immunostaining the liver tissues are shown in FIG. 1. In FIG. 1, the left panels correspond to normal livers, and the right panels correspond to the cirrhotic livers. In the normal livers, specifically in the livers of non-treated mice of the same age, CSPG (the top panel) and C4SPG (the second panel from the top) gave only weak positive signals within hepatic sinusoids, while C6SPG (the third panel from the top) gave no signal. Meanwhile, in the cirrhotic livers, specifically in the livers in which cirrhosis-like fibrosis was induced by carbon tetrachloride, all of CSPG (the top panel), C4SPG (the second panel from the top), and C6SPG (the third panel from the top) gave extensive positive signals mainly in sinusoids. The signals were significantly enhanced when compared to the normal livers. Heparan sulfate proteoglycan (HSPG) gave a strong positive signal in sinusoids of the normal livers. No significant difference in the signal intensity and distribution was found in the cirrhotic livers. Thus, CSPG was found to be dramatically increased in the liver of the cirrhosis model when compared with HSPG.

### Example 2

### Ability of Various Chondroitinases to Cleave Chondroitin Sulfate Accumulated in Mouse Cirrhotic Livers

In this Example, each type of chondroitinase was evaluated for its ability to cleave chondroitin sulfate (CSPG) in tissue sections from cirrhotic livers. Using the procedure described below, tissue sections of the cirrhotic livers were treated with chondroitinase, and after the enzyme reaction, residual CSPG in the tissue sections, specifically CSPG that was not cleaved in the chondroitinase treatment, was detected by the immunostaining method.

First, carbon tetrachloride (25 μl/100 g body weight, Sigma-Aldrich) was injected into the peritoneal cavities of C57BL/6JcL mice (female, five to six weeks old, CLEA Japan Inc.) twice a week for four weeks (eight times) to induce liver fibrosis. Then, carbon tetrachloride was additionally administered twice a week for two weeks (12 times in total) to induce cirrhosis. Mice with induced cirrhosis were sacrificed, and their livers were collected (cirrhotic livers).

Portions of second lobes from the collected livers were embedded in the OCT compound (Miles), an embedding medium for cryosectioning, and cryoblocks were prepared using liquid nitrogen. The cryoblocks were sliced into 6-μm sections using cryostat (Microm).

The resulting sections were fixed with acetone (Wako Pure Chemical Industries Ltd.) for ten minutes, and then washed with phosphate buffer. After the sections were incubated with Tris-HCl buffer (20 mM, pH8.0) at 37° C. for 15 minutes, 100 μl of chondroitinase ABC solution (Seikagaku Co., 5 U/ml), chondroitinase AC solution (Seikagaku Co., 5 U/ml), chondroitinase B solution (Seikagaku Co., 5 U/ml), or buffer alone (chondroitinase (−)) was added to the each section. The sections were covered and then incubated at 37° C. for one hour.

After the enzyme reaction, an anti-CSPG antibody (clone CS56, mouse monoclonal antibody, 10 μg/ml, Seikagaku Co.) was added and the sections were incubated at room temperature for one hour. After reaction using the Histofine Mouse Stain Kit (Nichirei), DAB substrate (Nichirei) was added thereto. The samples were observed under a light microscope (Leica Microsystems). Brown signals showing visible antibody binding were confirmed.

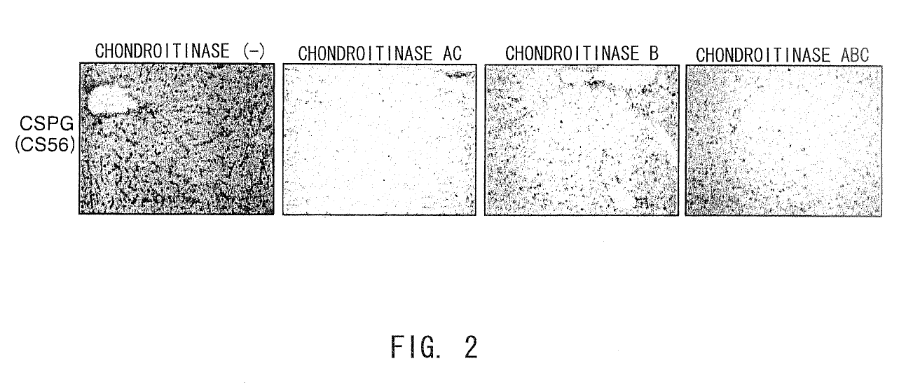

The result is shown in FIG. 2 (magnification, 200 fold). A significantly strong and extensive positive signal of CSPG (brown) was detected in the sections treated with buffer alone (specifically, sections without the enzyme treatment) (FIG. 2, the left most; chondroitinase (−)). The signal intensity was comparable to that detected in the cirrhotic liver shown in FIG. 1. Meanwhile, a positive CSPG signal was impaired and the positive area was decreased in the sections treated with chondroitinase AC as compared to the chondroitinase (−) sections (FIG. 2, the second from the left; chondroitinase AC). This suggests that indeed chondroitinase AC can cleave the CSPG accumulated in cirrhotic liver tissues. However, residual CSPG, which was not completely cleaved, was still detected in the sections treated with chondroitinase AC. In addition, a positive CSPG signal was further impaired and the positive area was also further reduced in the sections treated with chondroitinase B in comparison to the sections treated with chondroitinase AC. This suggests that chondroitinase B can cleave more of the CSPG accumulated in cirrhotic liver tissues. However, in the sections treated with chondroitinase B, residual CSPG was also detected along the pseudolobular structure. Thus, the chondroitinase AC or B treatment could not completely cleave the CSPG deposited in cirrhotic livers with advanced fibrosis.

In contrast to the results described above, in the sections treated with chondroitinase ABC, the positive CSPG signal (brown) was almost undetectable, and thus CSPG was clearly demonstrated to disappear when comparing the chondroitinase ABC-treated sections with the chondroitinase (−) sections. Specifically, chondroitinase ABC was demonstrated to be able to highly efficiently cleave CSPG deposited in lesions with advanced fibrosis such as cirrhosis.

### Example 3

### The Hepatic Fibrosis-Suppressing Effect of Chondroitinase ABC (Chase ABC) in Cirrhosis Mouse Models

Carbon tetrachloride (25 μl/100 g body weight, Sigma-Aldrich) was injected into the peritoneal cavities of C57BL/6JcL mice (female, five to six weeks old, CLEA Japan Inc.) twice a week for four weeks (eight times) to induce liver fibrosis. Then, carbon tetrachloride was additionally administered twice a week for two weeks (12 times in total).

Prior to further administration, an Alzet Osmotic Pump (Muromachi Kikai Co. Ltd.) pre-infused with 150 μl of chondroitinase ABC (Chase ABC) (20 U/ml, Sigma-Aldrich), chondroitinase AC (Chase AC) (20 U/ml, Sigma-Aldrich), chondroitinase B (Chase B) (20 U/ml, Sigma-Aldrich), or PBS alone was surgically implanted within the peritoneal cavity of each mouse. The respective groups of mice treated as described above were named Chase ABC enzyme group, Chase AC enzyme group, Chase B enzyme group, and control group. During two weeks of further administration of carbon tetrachloride, this treatment allowed constant infusion of a fixed volume of liquid from the implanted pump for all groups.

After further administration of carbon tetrachloride, mice from each group were sacrificed and their livers were collected. Cryosections of liver samples were prepared from the collected livers and analyzed by immunohistochemistry using the same procedure as described in Example 1.

In this analysis, a rabbit anti-mouse type III collagen antibody (at 1:250 dilution, Santa Cruz) was added as a primary antibody to the samples, and the samples were incubated at room temperature for one hour. Then, an alkaline phosphatase-labeled anti-rabbit IgG (at 1:200 dilution, Jackson ImmunoResearch) was added as a secondary antibody. After the sections were incubated at room temperature for 30 minutes, a chromogenic substrate for alkaline phosphatase (Vector Red, Vector Laboratory) was added thereto. The samples were observed under a light microscope (Leica Microsystems). Red signals showing visible antibody binding were confirmed.

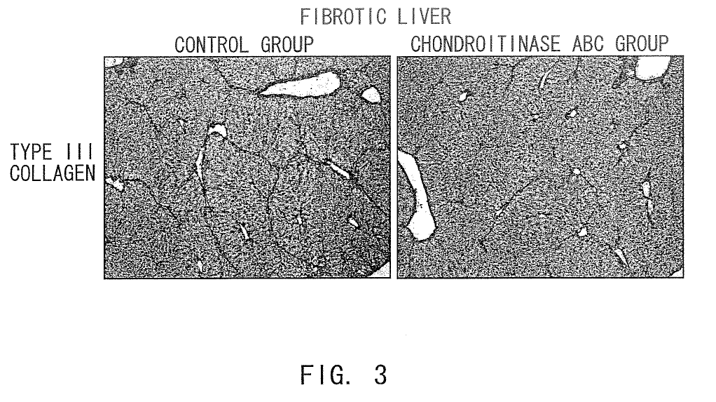

Examples of images obtained by immunostaining the liver tissues are shown in FIG. 3. It was found that type III collagen fibers detected using the rabbit anti-mouse type III collagen antibody elongated not only in general sinusoids but continuously through the central vein up to the portal vein, and pseudolobules were formed in some portions in the control group (the left panel in FIG. 3). This pattern was typical of cirrhosis. In contrast, in the Chase ABC enzyme group, elongation of type III collagen was very mild (the right panel in FIG. 3).

Based on the immunohistochemical staining carried out as described above, the degree of fibrosis of each sample was evaluated using fibrosis scores according to previous reports (Dai K, et al., World J. Gactroenterol. 2005, 31, 4822-4826; Hillebrandt S, et al., Nature Genetics 2005, 37, 835-843). The evaluation criteria for fibrosis scores are as follows: score 0, normal; score 1, a small fraction of collagen is found to elongate from the central vein; score 2, collagen evidently elongates from the central vein but to the extent that it does not cover the whole liver; score 3, collagen evidently elongates from the central vein to the extent that it covers the whole liver; score 4, diffuse collagen elongation is observed in the whole liver and pseudolobules are formed.

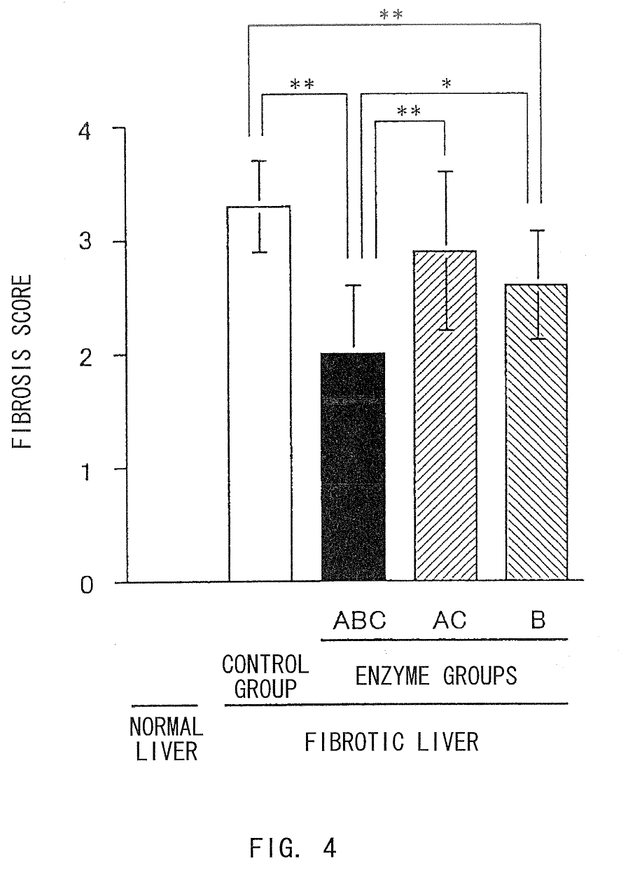

The result is shown as a graph in FIG. 4. Each bar represents mean value ±standard deviation for the fibrosis score in each group. As shown in FIG. 4, the score was 2.0±0.6 (n=6) in the Chase ABC enzyme group, 2.9±0.7 (n=6) in the Chase AC enzyme group, and 2.6±0.5 (n=6) in the Chase B enzyme group, while it was 5.3±0.4 (n=6) in the control group. When compared with the control group, reduction of fibrosis was found to be statistically significant in the Chase ABC enzyme group (p=0.00018, t test) and the Chase B enzyme group (=0.0029, t test). In particular, the fibrosis-suppressing effect was statistically significantly higher in the Chase ABC enzyme group than in the Chase AC enzyme group (p=0.0019, t test) and Chase B enzyme group (p=0.018, t test). Meanwhile, there was no statistical significant difference between the Chase AC enzyme and Chase B enzyme groups (p=0.15, t test). Normal livers collected from normal mice that were not administered with carbon tetrachloride were also analyzed in the same way by immunohistochemistry, and the fibrosis score was found to be 0. Thus, administration of chondroitinase ABC was demonstrated to exert a clinically superior effect of suppressing hepatic fibrosis.

### Example 4

### Effect of Chondroitinase ABC (Chase ABC) in Suppressing Deposition of Type I Collagen in Cirrhosis Mouse Models

In fibrotic diseases including cirrhosis, increase of type I collagen is assumed to be the most crucial factor in the formation of lesion-constituting fibers. Thus, immunostaining experiments for type I collagen were conducted using the liver tissue samples derived from each group prepared in Example 3.

A rabbit anti-mouse type I collagen antibody (at 1:100 dilution, Rockland) was added as a primary antibody to the sections prepared in Example 3, which were derived from the control group, Chase ABC enzyme group, Chase AC enzyme group, and Chase B enzyme group. The sections were incubated at room temperature for one hour. Then, an alkaline phosphatase-labeled anti-rabbit IgG (at 1:200 dilution, Jackson ImmunoResearch) was added as a secondary antibody. After the sections were incubated at room temperature for additional 30 minutes, a chromogenic substrate for alkaline phosphatase (Vector Red, Vector Laboratory) was added thereto. The samples were observed under a light microscope (Leica Microsystems). Red signals showing visible antibody binding were confirmed.

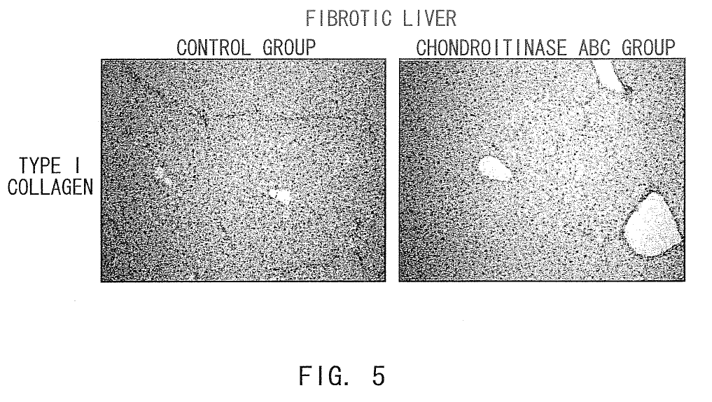

Examples of images obtained by immunostaining the liver tissues are shown in FIG. 5. As shown in FIG. 5, type I collagen was clearly detected along the formed pseudolobules in the control group. In contrast, the collagen was found only occasionally in the Chase ABC enzyme group.

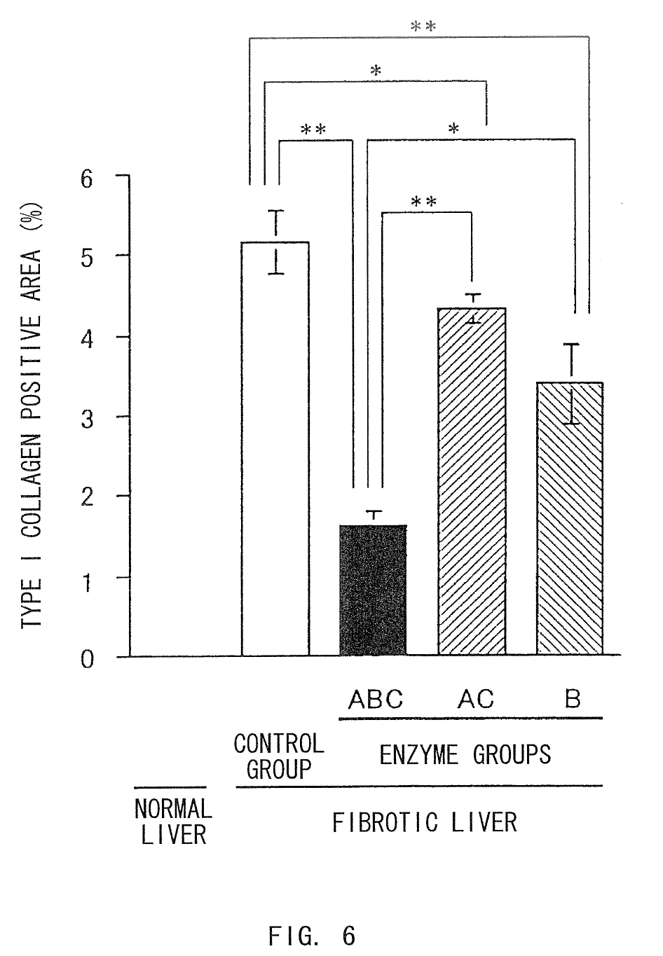

Then, based on the immunohistochemical staining carried out as described above, the type I collagen positive area was calculated using the Win ROOF image analysis software (Win ROOF, Mitani Co.). Areas of at least 15 mm² in the liver sections were measured, and the result was presented as a ratio (%) of the positive area to the liver area measured. The result is shown as a graph in FIG. 6. As also shown in FIG. 6, the percentage of type I collagen-positive area (%) was 1.61±0.29% in the Chase ABC enzyme group, 4.30±0.18% in the Chase AC enzyme group, 3.38±0.50% in the Chase B enzyme group, and 5.16±0.98% in the control group. The percentage of type I collagen-positive area (%) was significantly reduced in all the enzyme groups as compared with the control group (Chase ABC enzyme group, p=0.0018; Chase AC enzyme group, p=0.023; Chase B enzyme group, p=0.0059; all by t test). In particular, the percentage of type I collagen-positive area (%) in the Chase ABC enzyme group was also statistically significantly decreased as compared with those of the Chase AC enzyme group and the Chase B enzyme group (significant difference p=0.0008 when compared with the Chase AC enzyme group, significant difference p=0.003 when compared with the Chase B enzyme group, both by t test). Thus, the effect of Chase ABC enzyme in suppressing the deposition of type I collagen was demonstrated to be extremely strong. Meanwhile, there was no statistically significant difference between the Chase AC enzyme and Chase B enzyme groups (p=0.068, t test). The normal livers collected from normal mice that were not administered with carbon tetrachloride were also analyzed in the same way, and the percentage of type I collagen-positive area was found to be 0%. This result demonstrates that administration of chondroitinase ABC can significantly suppress the deposition of type I collagen in fibrotic livers.

### Example 5

### Effect of Chondroitinase ABC (Chase ABC) in Suppressing Deposition of Chondroitin Sulfate (CSPG) in Cirrhosis Mouse Models

To demonstrate that chondroitin sulfate (CSPG) deposited in fibrotic livers is degraded by administering Chase ABC, the liver tissue samples of the control group and the Chase ABC enzyme group prepared in Example 3 were analyzed by immunohistochemistry using anti-CSPG antibody, anti-C4SPG antibody, and anti-C6SPG antibody as primary antibodies by the same procedure as described in Example 1.

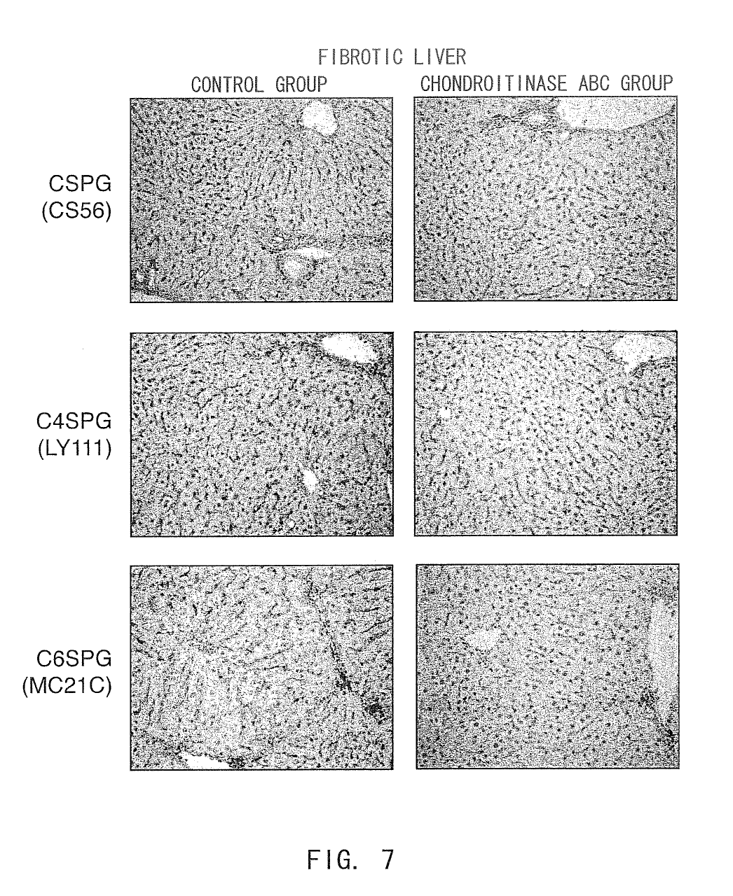

Examples of images obtained by immunostaining the liver tissues are shown in FIG. 7. In FIG. 7, the left panels correspond to the control group, and the right panels correspond to the Chase ABC enzyme group. As shown in FIG. 7, as is the case of cirrhotic livers in Example 1, all CSPG, C4SPG, and C6SPG gave clear positive signals in the control group. In contrast, evidently impaired signals were observed for all CSPG, C4SPG, and C6SPG in the Chase ABC enzyme group, as compared with the control group. In particular, the staining images for C6SPG (the bottom) showed that in the control group, a clear positive signal was detected surrounding the pseudolobules where the elongation of collagen was observed in Examples 4 and 5, while there was almost no such signal in the Chase ABC enzyme group.

The result described above demonstrates that CSPG deposited in fibrotic livers is degraded by administering chondroitinase ABC.

### Example 6

### Effect of Chondroitinase ABC (Chase ABC) in Suppressing Accumulation of Fibroblasts in Cirrhosis Mouse Models

As collagen-producing cells that enhance fibrosis, activated fibroblasts are a key player. Excessive accumulation, settlement, and activation of fibroblasts are assumed to exacerbate the pathological condition of fibrosis. Thus, the dynamics of fibroblasts in the cirrhosis model was examined by immunohistochemistry.

First, a rat anti-mouse fibroblast antibody (clone ER-TR7, at 1:500 dilution, BMA) was added as a primary antibody to the section samples derived from the control group and the Chase ABC enzyme group prepared in Example 3, and the sections were incubated at room temperature for one hour. Then, a peroxidase-labeled anti-rat IgG (at 1:200 dilution, Biosource) was added as a secondary antibody. After 30 minutes of incubation at room temperature, DAB substrate (Nichirei) was added thereto. The samples were observed under a light microscope (Leica Microsystems). Brown signals showing visible antibody binding were confirmed. The same immunohistochemical analysis was carried out using normal livers collected from normal mice that were not administered with carbon tetrachloride.

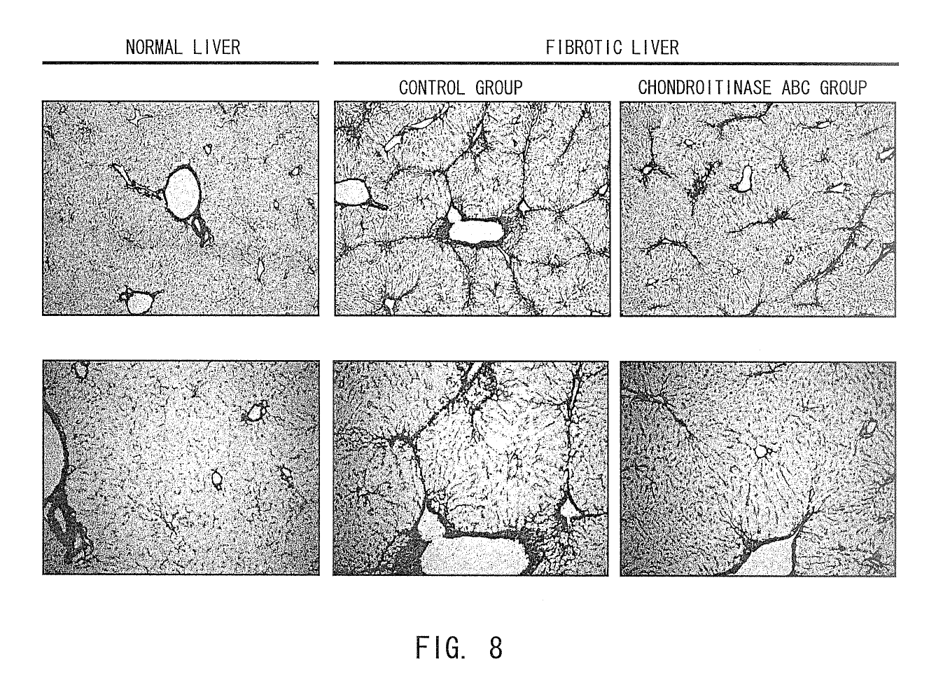

Examples of images obtained by immunostaining the liver tissues are shown in FIG. 8. In FIG. 8, the left most panels correspond to the normal group (normal livers), the middle panels correspond to the control group, and the right most panels correspond to the Chase ABC enzyme group. The upper panels are images at 100 fold magnification and the bottom panels are images at 200 fold magnification. Intense positive signals for fibroblast were distributed in areas surrounding the portal veins of liver in the non-treated normal mice. Furthermore, evident accumulation of fibroblasts along pseudolobules was seen in the control group (cirrhotic liver); however, such accumulation was very mild in the Chase ABC group. This finding indicates that excessive accumulation or settlement of fibroblasts in fibrotic livers was significantly suppressed by administering Chase ABC.

Furthermore, from the finding of this Example that fibroblasts accumulated along pseudolobules (fibrotic layer) in the cirrhotic livers and the finding of the above-described Example that a clear positive signal for CSPG was detected in areas surrounding pseudolobules in the cirrhotic livers of the control group, it is suggested that CSPG provides anchorage for fibroblasts in liver fibrosis.

### Example 7

### Effect of Chondroitinase ABC (Chase ABC) in Suppressing Accumulation of Macrophages in Cirrhosis Mouse Models

The major cell that produces fibroblast-activating factors is assumed to be macrophage. Thus, the dynamics of macrophages in the cirrhosis model were also examined by immunohistochemistry.

First, a rat anti-mouse macrophage antibody (clone F4/80, at 1:500 dilution, BMA) was added as a primary antibody to the section samples derived from the control group and the Chase ABC enzyme group prepared in Example 3, and the sections were incubated at room temperature for one hour. Then, a peroxidase-labeled anti-rat IgG (at 1:200 dilution, Biosource) was added as a secondary antibody. After 30 minutes of incubation at room temperature, DAB substrate (Nichirei) was added thereto. The samples were observed under a light microscope (Leica Microsystems). Brown signals showing visible antibody binding were confirmed. The same immunohistochemical analysis was carried out using normal livers collected from normal mice that were not administered with carbon tetrachloride.

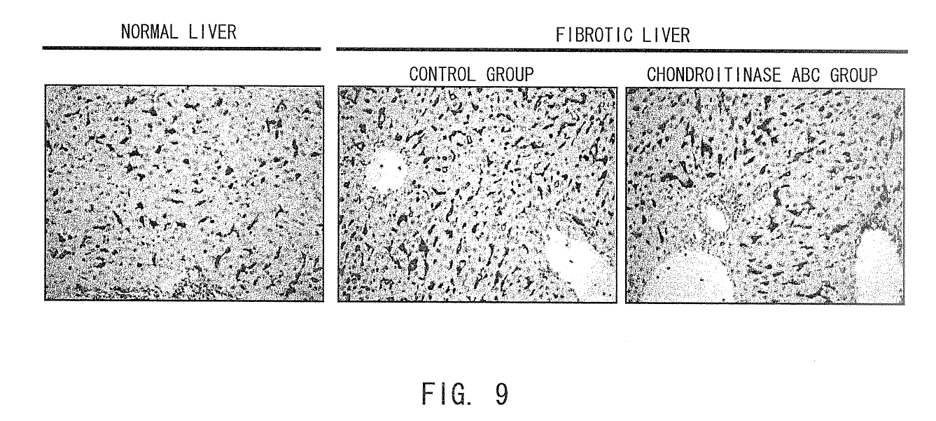

Examples of images obtained by immunostaining the liver tissues are shown in FIG. 9. In FIG. 9, the left most panels correspond to the normal group (normal liver), the middle panels correspond to the control group, and the right most panels correspond to the Chase ABC enzyme group. As shown in FIG. 9, positive signals were widely distributed in sinusoids in the normal livers, and the cells were assumed to be Kupffer cells. By contrast, the macrophage count was evidently increased in the cirrhotic livers of the control group, and many of the macrophages were detected near collagen that was found to surround pseudolobules in Examples 4 and 5. In contrast, the macrophage count was evidently reduced in the Chase ABC enzyme group as compared with the control group. Specifically, accumulation of macrophages in cirrhotic livers was demonstrated to be suppressed in the Chase ABC enzyme group. This result suggests that the progression of fibrosis was suppressed in the Chase ABC enzyme group as opposed to the formation of progressive fibrotic lesions in the control group (cirrhotic livers).

### Example 8

### Therapeutic Effect (Suppression of Fibroblast Infiltration) of Administering ADAMTS-4 Peptide in Cirrhosis Mouse Models

Carbon tetrachloride-induced cirrhosis mouse models were prepared by the same induction method as described in Example 1. The sequence of the ADAMTS-4 peptide used in the therapy was NH2-DRARSCAIVEDDGLQSAFTA-COOH (SEQ ID NO: 67) (synthesized as a peptide having positions 336 to 355 of mouse ADAMTS-4, which is a domain having metalloproteinase activity, GeneWorld Ltd., 1 μg/head). Vehicle (PBS) was used in the control group. The peptide or vehicle was administered into the peritoneal cavities four times in total, as is the case for the supplementary administration of carbon tetrachloride (a total of four times from the ninth to the twelfth time). After the supplementary administration, mice from both groups were sacrificed. Sections were prepared from their liver tissues and examined by immunohistochemistry.

Activated fibroblasts are a key player as collagen-producing cells that enhance fibrosis. Excessive accumulation, settlement, and activation of fibroblasts are assumed to exacerbate the pathological condition of fibrosis. Thus, the dynamics of fibroblasts in the cirrhosis model were examined by immunohistochemistry.

A rat anti-mouse fibroblast antibody (clone ER-TR7, at 1:500 dilution, BMA) was added as a primary antibody to the section samples derived from the control group and the ADAMTS-4-treated group, and the sections were incubated at room temperature for one hour. Then, a peroxidase-labeled anti-rat IgG (at 1:200 dilution, Biosource) was added as a secondary antibody. After 30 minutes of incubation at room temperature, DAB substrate (Nichirei) was added thereto. The samples were observed under a light microscope (Leica Microsystems). Brown signals showing visible antibody binding were confirmed.

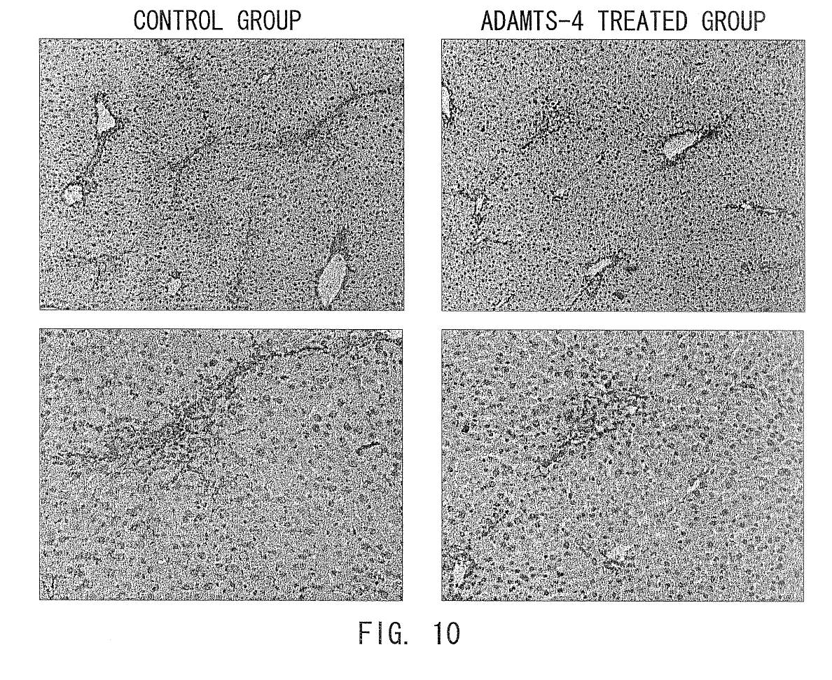

Examples of images obtained by immunostaining the liver tissues are shown in FIG. 10. In the control group (cirrhotic livers), fibroblasts were evidently demonstrated to accumulate along pseudolobules. In contrast, the degree of accumulation was very mild in the ADAMTS-4-treated group. This suggests that ADAMTS-4 administration significantly suppressed the excessive accumulation or settlement of fibroblasts in the fibrotic livers.

### Example 9

### Therapeutic Effect (Suppression of Type III Collagen) of Administering the ADAMTS-4 Peptide in Cirrhosis Mouse Models

Cryosections of liver samples were prepared and analyzed by immunohistochemistry using the same procedure as described in Example 8. A rabbit anti-mouse type III collagen antibody (at 1:250 dilution, Santa Cruz) was added as a primary antibody to the samples, and the samples were incubated at room temperature for one hour. Then, an alkaline phosphatase-labeled anti-rabbit IgG (at 1:200 dilution, Jackson ImmunoResearch) was added as a secondary antibody. After the sections were incubated at room temperature for 30 minutes, a chromogenic substrate for alkaline phosphatase (Vector Red, Vector Laboratory) was added thereto. The samples were observed under a light microscope (Leica Microsystems). Red signals showing visible antibody binding were confirmed.

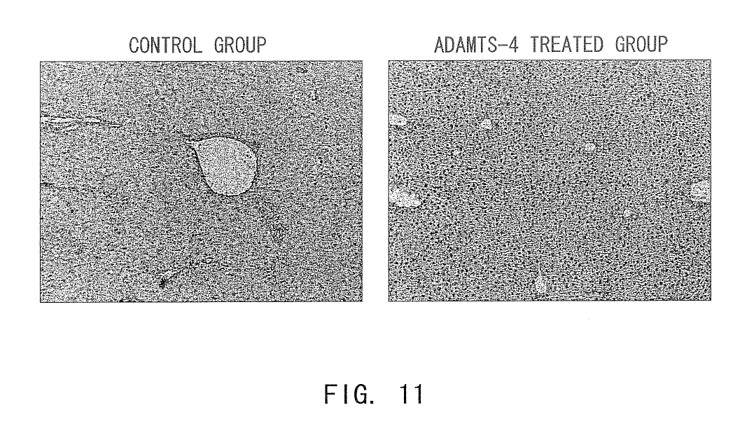

Examples of images obtained by immunostaining the liver tissues are shown in FIG. 11. It was found that type III collagen fibers detected using the rabbit anti-mouse type III collagen antibody elongated in sinusoids and continuously through the central vein up to the portal vein, and a portion of the fibers formed pseudolobules in the control group. This pattern was typical of cirrhosis. In contrast, in the ADAMTS-4 group, elongation of type III collagen was almost undetectable.

### Example 10

### Therapeutic Effect (Suppression in Fibrosis Score) of Administering the ADAMTS-4 Peptide in Cirrhosis Mouse Models

Based on the immunohistochemical staining carried out in Example 9, the degree of fibrosis in each sample was evaluated using fibrosis scores according to previous reports (Dai K, et al., World J. Gactroenterol. 2005, 31, 4822-4826; Hillebrandt S, et al., Nature Genetics 2005, 37, 835-843). The evaluation criteria for fibrosis scores are as follows: score 0, normal; score 1, a small fraction of collagen is found to elongate from the central vein; score 2, collagen evidently elongates from the central vein but to the extent that it does not cover the whole liver; score 3, collagen evidently elongates from the central vein to the extent that it covers the whole liver; score 4, diffuse collagen elongation is observed in the whole liver and pseudolobules are formed.

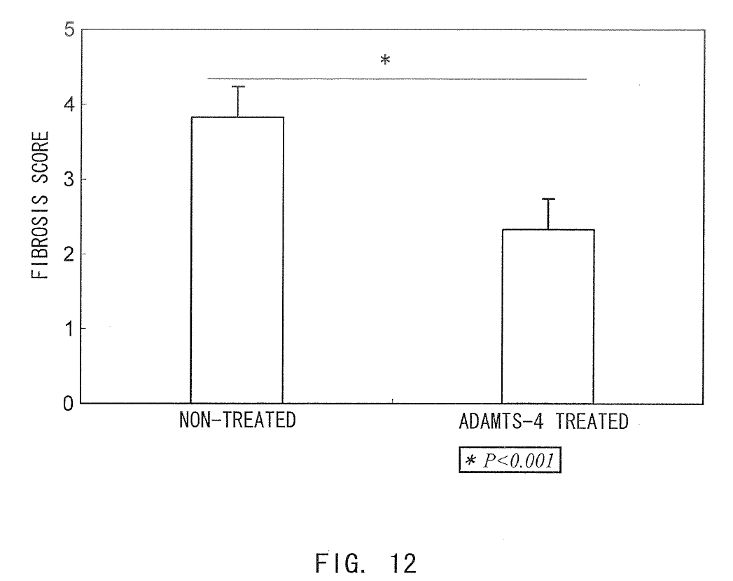

The result is shown as a graph in FIG. 12. Each bar represents mean value ±standard deviation for fibrosis score in each group. The score was 2.3±0.4 (n=6) in the ADAMTS-4-treated group, while it was 3.9±0.4 (n=6) in the control group. As compared with the control group, the fibrosis reduction was found to be statistically significant in the ADAMTS-4-treated group (p<0.001, t test). Thus, administration of the ADAMTS-4 peptide was demonstrated to exert a clinically superior effect of suppressing hepatic fibrosis.

### INDUSTRIAL APPLICABILITY

The hepatic fibrosis-suppressing agents of the present invention comprising chondroitinase ABC as an active ingredient suppress fibrosis of liver tissues and thus are effective for treating or preventing diseases accompanied by excessive fibrosis of liver tissues (fibrotic liver diseases). The hepatic fibrosis-suppressing agents of the present invention are highly useful in suppressing hepatic fibrosis, because they are efficacious in suppressing various symptoms associated with fibrosis in various ways, such as by suppressing elongation of type III collagen, suppressing deposition of type I collagen, suppressing accumulation of fibroblasts, and suppressing accumulation of macrophages in fibrotic liver tissues. Methods for treating or preventing fibrotic liver diseases by administering the hepatic fibrosis-suppressing agents of the present invention can effectively improve fibrotic lesions through medicinal therapy, and are potentially excellent therapeutic methods that are useful for improving patients' QOL.

All publications, patents, and patent applications cited herein are incorporated herein by reference in their entirety.

1. A hepatic fibrosis-suppressing agent comprising as an active ingredient a substance that inhibits the production or accumulation of a chondroitin sulfate proteoglycan.

2. The agent of claim 1, wherein the substance has an activity of promoting the degradation of a chondroitin sulfate proteoglycan.

3. The agent of claim 1, wherein the substance has an activity of inhibiting the synthesis of a chondroitin sulfate proteoglycan.

4. The agent of claim 1, wherein the substance has an activity of desulfating a chondroitin sulfate proteoglycan.

5. The agent of claim 1, wherein the substance has an activity of inhibiting the sulfation of a chondroitin sulfate proteoglycan.

6. The agent of claim 1, wherein the production or accumulation of a chondroitin sulfate proteoglycan is inhibited in liver.

7. The agent of claim 1, which is used for treating or preventing a fibrotic liver disease.

8. The agent of claim 7, wherein the fibrotic liver disease is a chronic liver disease.

9. The agent of claim 7, wherein the fibrotic liver disease is chronic hepatitis, liver fibrosis, cirrhosis, liver failure, or hepatocarcinoma.

10. A method of screening for a hepatic fibrosis-suppressing agent, which comprises selecting from a test sample a substance with an activity of inhibiting the production or accumulation of a chondroitin sulfate proteoglycan.

11. The screening method of claim 10, which comprises the step of selecting a substance with the activity of any one of:
(a) promoting the degradation of a chondroitin sulfate proteoglycan;
(b) inhibiting the synthesis of a chondroitin sulfate proteoglycan;
(c) desulfating a chondroitin sulfate proteoglycan; and
(d) inhibiting the sulfation of a chondroitin sulfate proteoglycan.

12. The screening method of claim 10, wherein the hepatic fibrosis-suppressing agent is used for treating or preventing a fibrotic liver disease.

13. A method of treating or preventing a disease accompanying hepatic fibrosis, which comprises administering to a patient in need thereof a substance that inhibits the production or accumulation of a chondroitin sulfate proteoglycan.
